SEQUENCE LISTING (65 sequences)

SEQ ID NO: 1 - DNA, 4308 bp, Streptococcus sobrinus
  source 1..4308: mol_type = unassigned DNA
atggttgacggcaaatactactattatgatcaggacggtaacgtaaagaagaatttcgcggtgagcgttggtgacaaaatctactacttcgatgaaactggtgcatataaggataccagcaaagtggacgccgacaagagcagcagcgcggttagccaaaacgcgaccatctttgcggcgaataaccgtgcgtacagcacctctgcaaagaattttgaagcggtggataactacctgaccgcagacagctggtatcgtccgaaatccatcctgaaggacggcaaaacctggaccgagagcggtaaggatgatttccgtccactgctgatggcatggtggcctgacaccgaaactaagcgcaactacgtgaactatatgaataaagtggtcggtattgacaagacgtacactgcggaaacgtcgcaagcggatttgaccgcagcggcggagctggttcaagcgcgtatcgagcagaagattaccagcgaaaacaacaccaaatggctgcgcgaagcaatctccgcgttcgttaagacgcagcctcagtggaacggcgagtccgaaaagccgtatgacgatcacttgcagaacggtgcgctgctgtttgataaccaaaccgacctgacgccagacacccaaagcaattaccgtttgctgaaccgtaccccgaccaatcagactggtagcctggatagccgttttacgtataatccgaatgacccgttgggcggctacgatttcttgctggcgaacgacgttgacaatagcaatccggtcgtccaggctgaacagttgaactggctgcattatctgctgaactttggctctatttacgctaacgatgccgacgccaattttgacagcattcgcgttgatgccgtcgataatgtcgatgctgatctgctgcaaatcagcagcgattacctgaaagcagcgtatggcatcgacaagaataacaagaatgcgaacaaccatgttagcatcgtcgaagcgtggagcgacaatgataccccgtatttgcacgacgatggcgataatctgatgaacatggacaacaaatttcgcctgtccatgctgtggagcctggcaaagccgctggacaaacgtagcggtttgaacccgctgattcacaatagcctggtggaccgcgaggtggacgatcgtgaagtggaaaccgtgccgtcctacagctttgctcgtgcacatgatagcgaggtgcaggacatcatccgtgacattatcaaggctgagattaacccaaatagctttggttatagcttcactcaagaagagatcgagcaagcctttaagatttacaacgaggatttgaagaaaacggacaagaaatacacccactacaatgtgccgctgagctacaccctgctgctgaccaacaagggcagcatcccgcgtgtgtactatggtgatatgttcaccgatgatggccaatacatggcaaacaagaccgtcaactacgacgcaatcgagagcctgctgaaagcccgtatgaaatatgtcagcggtggccaagcaatgcagaactatcaaattggtaatggcgagattttgaccagcgtgcgctatggtaaaggtgccctgaagcagagcgataagggtgacgcgacgacgcgcactagcggtgttggcgtggttatgggtaatcagccgaacttctccctggacggtaaagttgtggccctgaatatgggtgcggcccatgcgaatcaagaataccgtgcactgatggtcagcactaaagacggtgtggcaacttacgcaaccgatgctgacgcatccaaagcgggcctggtcaagcgtaccgacgagaacggctacctgtacttcctgaatgatgatctgaagggcgtcgcgaaccctcaggtttccggcttcttgcaagtgtgggttccagttggtgccgccgatgaccaggacattcgcgtcgccgccagcgacacggcgagcacggatggtaaaagcctgcatcaagatgcggcgatggacagccgcgtcatgtttgagggtttcagcaattttcaatccttcgcgaccaaagaagaagaatacacgaatgttgttatcgcgaacaatgtcgataagttcgttagctggggtatcaccgattttgaaatggctccgcagtatgttagcagcaccgacggtcagttcttggacagcgtcatccagaatggctatgcgtttactgatcgctatgatctgggtatgtccaaggcgaacaagtatggcacggcagaccaactggttaaggcaatcaaagccctgcacgctaaaggcctgaaagttatggcggactgggtcccggatcaaatgtacacctttccaaaacaggaagttgtgaccgttacccgcaccgacaaattcggtaaaccgatcgccggctctcaaatcaatcacagcttgtatgtgaccgacaccaaatccagcggcgacgactaccaagcgaagtacggcggtgccttcctggatgaactgaaagaaaagtacccggaactgttcacgaaaaagcaaattagcacgggccaagcgattgatccgagcgtgaaaatcaagcagtggagcgcaaaatacttcaatggttcgaatatcctgggtcgcggtgcggactatgtgctgagcgaccaggtcagcaataagtatttcaacgtggcgagcgacaccttgttcctgccgtccagcctgctgggcaaggtcgtggagagcggcattcgttacgacggcaagggttacatctacaacagctccgcgaccggcgatcaggtcaaagcgtctttcattacggaagccggtaacctgtattacttcggcaaagacggttacatggttactggtgcccagacgattaatggcgccaactacttcttcctggaaaacggtacggcactgcgtaatacgatttacaccgatgctcaaggtaatagccactattacgcgaatgatggcaaacgctatgaaaatggctatcaacagttcggtaacgattggcgctactttaaagatggtaacatggcagtcggcctgaccacggttgatggcaacgtgcaatactttgacaaagacggcgtccaggcaaaggataagattatcgtcacccgtgatggcaaggtccgttacttcgatcagcacaacggtaacgcggcgaccaacacgttcattgctgataaaactggccattggtattacctgggtaaagatggcgtcgcggtgactggcgcccagaccgtcggcaaacaaaaactgtacttcgaggccaacggtcaacaagttaaaggtgactttgttacgtccgatgagggcaaactgtatttctatgacgttgattctggtgacatgtggacggacaccttcatcgaggataaggcgggcaactggttctatttgggcaaggatggtgcggcagttacgggtgcccaaacgattcgcggtcagaagctgtacttcaaggccaatggtcaacaggtcaagggtgacattgttaagggcaccgacggtaaaatccgctactatgatgcaaaatccggtgaacaggtgttcaacaaaacggtgaaagctgcggatggcaaaacgtatgttatcggtaatgatggtgtcgcggtggaccctagcgtggttaaaggtcaaacctttaaggacgcttcgggcgctctgcgtttctacaacttgaagggtcaactggtcactggcagcggctggtatgaaaccgcgaaccatgactgggtttacattcagtccggcaaggcactgaccggcgaacagaccattaacggtcaacacctgtatttcaaagaagatggtcaccaagtcaagggtcagttggtcacgggcaccgatggtaaagtgcgttactatgacgccaacagcggtgaccaagcattcaacaagagcgtcactgtgaatggtaaaacctattactttggcaacgatggtacggcgcagactgctggcaacccgaagggtcagacgttcaaggatggctccgacatccgtttttactctatggaaggccaactggtgaccggctcgggttggtacgagaacgcgcaaggccagtggctgtatgtgaaaaacggtaaggtgctgactggtctgcaaaccgttggcagccagcgtgtttacttcgacgagaatggtattcaggccaagggcaaagcagtgcgtaccagcgatggcaaaattcgttatttcgacgaaaacagcggcagcatgatcacgaatcaatggaagttcgtctatggtcagtattactactttggtaacgacggtgcacgtatttaccgtggttggaactaa

SEQ ID NO: 2 - protein, 1435 aa, Streptococcus sobrinus
MVDGKYYYYDQDGNVKKNFAVSVGDKIYYFDETGAYKDTSKVDADKSSSAVSQNATIFAANNRAYSTSAKNFEAVDNYLTADSWYRPKSILKDGKTWTESGKDDFRPLLMAWWPDTETKRNYVNYMNKVVGIDKTYTAETSQADLTAAAELVQARIEQKITSENNTKWLREAISAFVKTQPQWNGESEKPYDDHLQNGALLFDNQTDLTPDTQSNYRLLNRTPTNQTGSLDSRFTYNPNDPLGGYDFLLANDVDNSNPVVQAEQLNWLHYLLNFGSIYANDADANFDSIRVDAVDNVDADLLQISSDYLKAAYGIDKNNKNANNHVSIVEAWSDNDTPYLHDDGDNLMNMDNKFRLSMLWSLAKPLDKRSGLNPLIHNSLVDREVDDREVETVPSYSFARAHDSEVQDIIRDIIKAEINPNSFGYSFTQEEIEQAFKIYNEDLKKTDKKYTHYNVPLSYTLLLTNKGSIPRVYYGDMFTDDGQYMANKTVNYDAIESLLKARMKYVSGGQAMQNYQIGNGEILTSVRYGKGALKQSDKGDATTRTSGVGVVMGNQPNFSLDGKVVALNMGAAHANQEYRALMVSTKDGVATYATDADASKAGLVKRTDENGYLYFLNDDLKGVANPQVSGFLQVWVPVGAADDQDIRVAASDTASTDGKSLHQDAAMDSRVMFEGFSNFQSFATKEEEYTNVVIANNVDKFVSWGITDFEMAPQYVSSTDGQFLDSVIQNGYAFTDRYDLGMSKANKYGTADQLVKAIKALHAKGLKVMADWVPDQMYTFPKQEVVTVTRTDKFGKPIAGSQINHSLYVTDTKSSGDDYQAKYGGAFLDELKEKYPELFTKKQISTGQAIDPSVKIKQWSAKYFNGSNILGRGADYVLSDQVSNKYFNVASDTLFLPSSLLGKVVESGIRYDGKGYIYNSSATGDQVKASFITEAGNLYYFGKDGYMVTGAQTINGANYFFLENGTALRNTIYTDAQGNSHYYANDGKRYENGYQQFGNDWRYFKDGNMAVGLTTVDGNVQYFDKDGVQAKDKIIVTRDGKVRYFDQHNGNAATNTFIADKTGHWYYLGKDGVAVTGAQTVGKQKLYFEANGQQVKGDFVTSDEGKLYFYDVDSGDMWTDTFIEDKAGNWFYLGKDGAAVTGAQTIRGQKLYFKANGQQVKGDIVKGTDGKIRYYDAKSGEQVFNKTVKAADGKTYVIGNDGVAVDPSVVKGQTFKDASGALRFYNLKGQLVTGSGWYETANHDWVYIQSGKALTGEQTINGQHLYFKEDGHQVKGQLVTGTDGKVRYYDANSGDQAFNKSVTVNGKTYYFGNDGTAQTAGNPKGQTFKDGSDIRFYSMEGQLVTGSGWYENAQGQWLYVKNGKVLTGLQTVGSQRVYFDENGIQAKGKAVRTSDGKIRYFDENSGSMITNQWKFVYGQYYYFGNDGARIYRGWN

SEQ ID NO: 3 - DNA, 4026 bp, Streptococcus salivarius
  source 1..4026: mol_type = unassigned DNA
atgatcgacggcaaatactattatgttaatgaggacggtagccacaaagaaaactttgcgattacggttaatggtcaactgctgtatttcggtaaggacggcgcactgacctctagcagcacttacagctttaccccaggtacgacgaacatcgtggatggcttttctatcaacaaccgcgcgtatgactccagcgaagcgtcctttgaactgattgatggctacttgactgccgactcctggtatcgtccggcttccatcatcaaggacggtgtcacgtggcaggccagcaccgcagaggactttcgcccgctgctgatggcgtggtggccaaacgtggatacccaggtgaactatctgaactacatgtctaaagtgtttaacctggacgcaaagtatagcagcaccgataaacaagagactctgaaggttgcagctaaggatattcagattaagatcgagcagaaaattcaggcggagaaaagcacccaatggctgcgcgaaacgatcagcgcttttgtgaaaacccaaccacagtggaacaaagagactgagaattactcgaaaggtggtggtgaggatcatctgcaaggcggtgcactgctgtacgtgaatgatagccgtaccccgtgggcaaatagcgattatcgccgcctgaaccgcaccgctaccaatcaaacgggtacgattgacaagtccattctggacgagcagagcgacccaaatcacatgggcggtttcgacttcctgctggcgaatgatgttgacctgtccaacccggttgtgcaggcagagcagctgaaccagattcactacttgatgaattggggctctatcgtgatgggtgacaaagacgcaaactttgatggtatccgtgtcgatgcagttgacaacgtcgatgccgacatgctgcaactgtataccaactacttccgtgaatactacggtgttaacaaaagcgaagcgaacgcactggcgcacattagcgttttggaagcgtggagcttgaatgataatcactacaacgacaaaaccgatggtgcagcattggcgatggagaataagcagcgtctggcgctgctgtttagcctggctaaaccgattaaagagcgcaccccggcagtgagcccgctgtataacaacaccttcaatacgacccaacgcgatgagaaaaccgactggatcaataaagacggttctaaggcctataacgaggatggtactgtgaagcagagcaccattggtaagtacaatgaaaaatatggtgatgcatcgggcaattatgtgttcatccgtgcccatgataacaatgtccaagacatcattgcggagatcattaagaaagaaatcaacccgaaaagcgatggtttcaccatcactgacgccgaaatgaaacaagcgttcgagatttacaataaggacatgctgagcagcgacaagaagtacaccctgaataacatcccggcagcttatgccgtgatgttgcagaacatggaaacgattacccgtgtctattatggtgacctgtacaccgacgacggccactacatggaaaccaagtccccgtattacgacaccatcgttaacctgatgaaaagccgtatcaagtacgtcagcggtggccaggcccaacgtagctactggctgccgaccgacggcaagatggacaatagcgacgttgagctgtatcgcaccaacgaagtgtataccagcgtccgttacggtaaagacattatgaccgcgaacgataccgagggtagcaagtacagccgcaccagcggccaggtcaccctggttgcaaacaacccgaagctgaccctggaccagagcgcgaagctgaatgtggaaatgggtaagattcacgcgaatcagaaataccgtgccctgattgtgggcacggctgacggtatcaagaatttcaccagcgacgcagatgctatcgcggcaggctacgtgaaagaaaccgactccaatggcgttctgacttttggcgctaatgacatcaaaggttatgaaaccttcgacatgtccggctttgttgctgtttgggtgccggtcggcgcgagcgatgatcaggacattcgtgtcgctcctagcactgaggccaagaaagagggtgaattgaccctgaaagcgaccgaagcatacgattcccagctgatctatgaaggttttagcaattttcaaaccatcccggatggtagcgacccgagcgtgtacaccaatcgcaagatcgcagagaacgtggacctgttcaagtcctggggtgttacctcgtttgaaatggcaccgcagttcgtttccgcagatgatggcacttttctggactctgtgatccaaaacggctatgcgtttgccgatcgttacgatttggcgatgagcaagaacaacaaatacggcagcaaagaggacttgcgtgacgcgctgaaagccctgcataaagcaggcatccaggcgattgcagactgggtcccggaccagatttatcagttgccgggcaaagaagtggtcacggcgactcgcaccgacggcgcaggccgtaaaatcgcggacgcgatcattgatcatagcctgtacgttgcgaacactaagagcagcggcaaagattaccaggcgaagtacggtggtgagttcttggcggagctgaaggccaagtacccggagatgttcaaagtgaacatgatttctaccggcaaaccgattgatgacagcgtcaaactgaaacagtggaaagcagaatactttaacggcaccaacgtcttggagcgcggtgtgggttatgtcctgagcgatgaagccacgggtaaatactttaccgtcacgaaggatggcaacttcattccgttgcagctgacgggtaatgagaaagtcgtgaccggctttagcaatgatggcaaaggtatcacctacttcggtacgagcggcactcaagcgaaatctgcgttcgttacgttcaatggtaatacttactattttgacgctcgtggtcacatggttacgaacggcgagtattcgccgaacggtaaggatgtttaccgtttcctgccgaatggtattatgctgtctaacgctttttacgttgatgcaaatggtaacacgtacctgtacaacagcaagggccaaatgtacaaaggcggttacaccaaatttgacgttaccgaaacggacaaagatggtaaggaaagcaaggtggtgaagtttcgttactttacgaacgaaggtgtcatggcaaaaggcgttaccgtgattgacggcttcacgcaatactttggtgaagatggtttccaagcgaaagacaagctggtcacgttcaagggcaagacgtactacttcgatgcacacaccggcaatgcgatcaaggacacctggcgtaatatcaatggcaagtggtatcatttcgacgcgaacggcgttgcagcgaccggcgctcaggtcatcaatggccaaaaactgtatttcaacgaggacggcagccaagtgaaaggcggtgttgtcaaaaacgcggacggtacgtattctaaatacaaagagggttctggtgaactggttaccaacgagttcttcacgacggatggcaatgtttggtactacgcaggcgcgaatggcaagaccgttacgggtgcccaggtgattaacggccaacacctgtacttcaatgcggacggttcgcaagtgaagggcggtgtggtcaagaacgcggatggcacctatagcaaatatgatgcgtctaccggcgaacgcctgaccaatgagtttttcaccacgggtgataacaactggtactacattggcgcaaacggcaagagcgtgacgggcgaggtcaagatcggtgacgatacctatttctttgccaaagatggcaagcaagttaagggtcaaactgtcagcgcgggtaacggtcgtattagctactactatggtgatagcggtaagcgtgcggtgagcacttggatcgaaatccaaccgggtgtttatgtctacttcgacaagaacggcattgcctatccgcctcgtgtgctgaattaa

SEQ ID NO: 4 - protein, 1341 aa, Streptococcus salivarius [sequence omitted]

SEQ ID NO: 5 - DNA, 3744 bp, Streptococcus salivarius
  source 1..3744: mol_type = unassigned DNA
atgccaagccacattaagaccatcaacggcaaacaatactacgtggaggatgacggtacgattcgcaagaattacgtcctggagcgtatcggtggcagccaatactttaatgcagaaaccggtgaactgtctaatcagaaagagtatcgtttcgacaaaaatggtggtactggtagcagcgcggacagcacgaacaccaacgtgactgtgaacggtgacaaaaacgcattttacggtaccacggacaaagacattgagctggtcgacggctatttcaccgcgaacacctggtatcgcccgaaagaaatcctgaaagacggcaaagaatggaccgccagcacggagaacgataaacgcccgctgctgaccgtctggtggcctagcaaagcaatccaggcgtcttatctgaactacatgaaagagcaaggcctgggtaccaaccaaacgtacacgagcttctccagccaaacccaaatggatcaagcagccctggaagtgcaaaagcgtattgaagagcgcatcgcacgcgagggcaataccgactggctgcgcacgaccatcaagaacttcgtgaaaacccaaccgggttggaacagcacctctgaaaatctggacaataatgatcatctgcaaggtggcgccctgctgtacaataacgactcccgcacgagccacgcgaacagcgactatcgcctgctgaatcgtacgccgaccagccagaccggcaaacacaatccgaaatacaccaaagataccagcaatggtggtttcgaatttctgctggcgaacgacatcgataactctaatccggcggttcaagcagagcaactgaactggctgcattacattatgaacatcggtaccatcacgggcggttctgaggatgaaaacttcgacggcgttcgtgttgacgctgtggataatgtgaatgcggatctgctgcaaatcgcgagcgactatttcaaagcaaaatacggtgctgatcaaagccaagatcaggcgatcaaacacttgagcatcctggaagcgtggtcccataacgacgcctactataacgaagataccaaaggcgcgcagttgccgatggatgatccgatgcacctggctctggtctactcgctgctgcgtccgatcggcaatcgcagcggtgtggaaccgctgatttccaacagcctgaatgaccgtagcgagtccggtaagaacagcaaacgtatggcgaactacgcgttcgtacgcgcgcatgatagcgaggtgcaatcgattattggccagatcatcaaaaacgagatcaatccgcaaagcaccggtaatacgttcaccctggatgagatgaagaaagcgtttgagatttacaacaaggatatgcgtagcgcgaataagcagtatacgcagtacaacatcccgagcgcgtatgcgttgatgctgacccacaaggataccgttccgcgtgtgtattacggtgatatgtatacggacgacggtcagtacatggcgcaaaagagcccatactatgatgcgatcgaaacgctgctgaaaggtcgcatccgctatgccgcaggtggtcaggacatgaaggtcaactatattggttacggtaacactaacggctgggatgctgcgggcgtgctgaccagcgtacgttatggcacgggcgcaaatagcgccagcgatacgggtaccgccgaaacgcgtaatcaaggtatggcagtgattgttagcaaccaaccggcgctgcgtctgactagcaatttgaccattaacatgggtgccgcacaccgtaatcaggcttaccgtccgctgctgctgacgaccaacgatggcgtcgcgacctatttgaacgatagcgatgcgaatggtatcgttaagtacaccgacggtaatggtaatctgaccttctccgcaaacgagattcgtggcatccgtaacccgcaagttgatggctatctggccgtctgggttccggtaggtgcgtcggagaatcaggatgttcgtgtggcgccgagcaaagagaagaacagctccggtctggtttacgagagcaatgctgccctggatagccaagttatctacgaaggcttcagcaacttccaggacttcgttcagaatccgagccagtataccaacaaaaagattgcagagaatgcaaatttgttcaaatcctggggtattaccagctttgaatttgcgccgcagtacgtgagctcggatgatggtagcttcctggacagcgttattcagaacggttatgcgtttacggaccgctacgacattggtatgagcaaagacaacaaatatggttcgctggcggatttgaaggcagcactgaagagcttgcatgccgttggtattagcgcaatcgcggattgggttcctgatcagatctacaatctgccaggcgacgaggtcgtcaccgcaacccgcgttaacaactacggcgaaaccaaagatggtgcaatcattgatcactctttgtacgcggccaaaacccgtacttttggtaacgactaccagggtaagtatggtggtgcgttcctggacgagctgaaacgtctgtatccgcagatctttgaccgcgttcagatttctaccggtaagcgcatgaccacggacgagaagatcacccaatggtctgcaaagtatatgaacggtacgaacatcttggaccgtggctctgaatacgttttgaagaatggtctgaatggttactatggcaccaatggtggcaaagtttcgctgccgaaagttgtgggtagcaatcaaagcacgaatggcgacaatcaaaacggcgacggtagcggcaagtttgaaaagcgtctgttcagcgtgcgttaccgttataacaatggccagtacgcgaaaaatgcctttatcaaagataacgacggcaatgtttactatttcgacaatagcggtcgtatggctgtcggtgagaaaacgattgacggcaagcagtacttcttcctggctaatggcgttcagctgcgtgacggctaccgtcaaaatcgtcgcggtcaggtgttttactacgaccagaatggtgtgctgaacgcaaacggtaaacaagacccgaagcctgacaacaataacaatgcgagcggccgtaatcaattcgtccagatcggtaacaacgtgtgggcgtattatgatggcaatggtaaacgtgtcaccggtcaccagaacatcaacggtcaggagttgtttttcgataacaacggtgtccaggttaagggtcgtacggtgaatgagaacggtgcaattcgctactatgacgcgaatagcggtgagatggcacgcaatcgtttcgcggagattgaaccgggcgtctgggcatactttaacaatgacggcaccgcagtgaagggttctcagaatatcaatggtcaagacctgtacttcgaccagaacggtcgtcaggtcaagggtgcgctggccaatgttgatggcaacctgcgctattacgacgttaacagcggtgagctgtaccgtaatcgtttccacgaaatcgacggcagctggtattactttgatggtaacggtaatgcggtgaagggtatggtcaatatcaacggccaaaatctgttgtttgacaataacggcaaacagattaagggtcatctggtccgcgtcaacggcgtcgtgcgctattttgatccgaactctggtgaaatggcggttaatcgttgggttgaggtgagcccaggttggtgggtttactttgacggtgaaggtcgtggtcagatctaa

SEQ ID NO: 6 - protein, 1247 aa, Streptococcus salivarius
MPSHIKTINGKQYYVEDDGTIRKNYVLERIGGSQYFNAETGELSNQKEYRFDKNGGTGSSADSTNTNVTVNGDKNAFYGTTDKDIELVDGYFTANTWYRPKEILKDGKEWTASTENDKRPLLTVWWPSKAIQASYLNYMKEQGLGTNQTYTSFSSQTQMDQAALEVQKRIEERIAREGNTDWLRTTIKNFVKTQPGWNSTSENLDNNDHLQGGALLYNNDSRTSHANSDYRLLNRTPTSQTGKHNPKYTKDTSNGGFEFLLANDIDNSNPAVQAEQLNWLHYIMNIGTITGGSEDENFDGVRVDAVDNVNADLLQIASDYFKAKYGADQSQDQAIKHLSILEAWSHNDAYYNEDTKGAQLPMDDPMHLALVYSLLRPIGNRSGVEPLISNSLNDRSESGKNSKRMANYAFVRAHDSEVQSIIGQIIKNEINPQSTGNTFTLDEMKKAFEIYNKDMRSANKQYTQYNIPSAYALMLTHKDTVPRVYYGDMYTDDGQYMAQKSPYYDAIETLLKGRIRYAAGGQDMKVNYIGYGNTNGWDAAGVLTSVRYGTGANSASDTGTAETRNQGMAVIVSNQPALRLTSNLTINMGAAHRNQAYRPLLLTTNDGVATYLNDSDANGIVKYTDGNGNLTFSANEIRGIRNPQVDGYLAVWVPVGASENQDVRVAPSKEKNSSGLVYESNAALDSQVIYEGFSNFQDFVQNPSQYTNKKIAENANLFKSWGITSFEFAPQYVSSDDGSFLDSVIQNGYAFTDRYDIGMSKDNKYGSLADLKAALKSLHAVGISAIADWVPDQIYNLPGDEVVTATRVNNYGETKDGAIIDHSLYAAKTRTFGNDYQGKYGGAFLDELKRLYPQIFDRVQISTGKRMTTDEKITQWSAKYMNGTNILDRGSEYVLKNGLNGYYGTNGGKVSLPKVVGSNQSTNGDNQNGDGSGKFEKRLFSVRYRYNNGQYAKNAFIKDNDGNVYYFDNSGRMAVGEKTIDGKQYFFLANGVQLRDGYRQNRRGQVFYYDQNGVLNANGKQDPKPDNNNNASGRNQFVQIGNNVWAYYDGNGKRVTGHQNINGQELFFDNNGVQVKGRTVNENGAIRYYDANSGEMARNRFAEIEPGVWAYFNNDGTAVKGSQNINGQDLYFDQNGRQVKGALANVDGNLRYYDVNSGELYRNRFHEIDGSWYYFDGNGNAVKGMVNINGQNLLFDNNGKQIKGHLVRVNGVVRYFDPNSGEMAVNRWVEVSPGWWVYFDGEGRGQI

SEQ ID NO: 7 - DNA, 4434 bp, Streptococcus salivarius
  source 1..4434: mol_type = unassigned DNA
atggacgaaacgcaggataagaccgtgacgcagagcaacagcggcaccaccgcttccctggtcactagccctgaagccacgaaagaggcggacaaacgcacgaacactaaagaggccgacgttctgacgcctgcaaaagaaacgaacgcagtcgagactgcgaccaccactaacacccaggcgacggcggaggccgccacgaccgcgaccaccgcggacgtcgcggtggctgcggtgccgaacaaagaagcggtcgttaccacggatgctccggcggtcacgaccgagaaagcggaagaacagccggctaccgttaaagcagaagtcgtcaatacggaagtgaaagcgccggaagcggctctgaaagacagcgaggttgaggcagcgctgagcctgaagaacatcaagaacattgatggcaagtattactatgttaatgaggatggcagccacaaagagaatttcgctattaccgtgaatggccagctgctgtactttggtaaagacggtgcgctgacgtcctctagcacgtattcttttaccccaggcactaccaatatcgtggacggttttagcattaacaaccgcgcttacgacagcagcgaggcgagctttgagctgatcgacggttacttgaccgcagacagctggtatcgtccggctagcatcatcaaagatggtgttacgtggcaagcgtccaccgccgaggattttcgtccgctgctgatggcatggtggccgaatgtggatacgcaggtgaactatttgaattacatgtccaaagttttcaacctggacgcgaaatactctagcaccgacaaacaggaaaccctgaaagtggcagcaaaagacattcaaatcaagattgaacaaaagattcaagcggagaagagcacgcagtggctgcgtgaaactatcagcgcctttgtgaaaacccagccgcagtggaacaaagaaaccgagaattacagcaagggtggtggtgaggaccacctgcaaggtggcgcactgctgtatgttaacgacagccgtaccccttgggcgaatagcgattaccgtcgtctgaatcgcaccgcaaccaatcagacgggcacgatcgataagtctattctggacgagcagtctgacccaaaccacatgggcggtttcgactttctgctggcgaacgacgtcgacctgagcaatccggtcgtgcaggctgagcagctgaatcaaatccactatctgatgaattggggttccattgtgatgggtgacaaggatgcgaactttgacggcattcgtgtcgatgcagttgacaacgtggacgcggacatgttgcaactgtataccaattacttccgtgagtactacggtgtgaacaagagcgaagctaacgcactggctcacatcagcgttctggaggcgtggagcctgaatgataatcattacaatgacaagaccgatggtgcggcactggcaatggagaataagcaacgtctggcgctgttgttttcgttggcgaaaccgatcaaagagcgtaccccggcagtgagcccgctgtataacaacaccttcaataccacccagcgtgatgaaaagaccgattggattaacaaagacggtagcaaggcttacaacgaagatggcacggtcaaacaatcgaccatcggtaagtacaacgagaaatacggtgacgcatccggtaactacgttttcatccgtgcccacgataacaacgtccaggacatcatcgccgagatcatcaagaaagagatcaacccgaaaagcgacggcttcaccatcaccgacgccgaaatgaagcaagcctttgaaatctataacaaagatatgctgtcgagcgacaaaaagtataccctgaataacattccggcagcgtatgccgtgatgttgcagaatatggaaacgattacccgcgtctattacggtgatctgtatacggacgacggtcactacatggaaaccaaatctccgtattacgataccatcgtgaatttgatgaagagccgtatcaagtatgtttcgggtggccaggcgcaacgtagctattggctgccgaccgacggtaagatggacaatagcgacgttgagctgtaccgcacgaatgaggtttacacgagcgtgcgctatggtaaggatatcatgaccgctaatgataccgaaggctctaagtattcccgcaccagcggccaagtcaccttggtcgcgaacaatccgaagctgaatctggaccaaagcgccaagttgaatgtggagatgggcaaaatccatgcgaatcagaagtatcgcgcactgattgtcggcactgcggacggcattaagaactttacttccgacgcggacgccattgcagcgggttatgtgaaagaaaccgatagcaacggcgtgctgaccttcggtgctaacgacattaagggctacgaaacgtttgatatgagcggtttcgtggcggtgtgggttccggtgggtgcatctgacaatcaggacattcgtgttgcgccgagcaccgaggcaaagaaagaaggtgagctgaccttgaaggcgacggaagcgtatgatagccagctgatttacgaaggctttagcaatttccagacgatcccagatggcagcgatccgtccgtgtatacgaaccgcaagattgcggagaacgtggatctgttcaaaagctggggtgtcaccagctttgagatggcaccgcaatttgtctcggcggatgatggcacctttctggatagcgttattcagaatggctacgccttcgccgaccgttatgacctggccatgtccaagaacaacaagtatggtagcaaagaggacctgcgtgatgcactgaaagcactgcataaggcgggtattcaagctatcgcagactgggttccagaccagatctaccagctgccgggcaaagaagttgtcaccgccacccgtacggatggtgctggccgtaagatcgcagacgcgattatcgaccattctctgtatgttgcaaacagcaaaagcagcggcaaagattatcaagcaaagtacggtggcgagttcctggccgagctgaaagccaaatacccggaaatgttcaaagttaacatgattagcacgggtaagccgattgatgactccgtgaaattgaagcaatggaaagccgagtacttcaatggcaccaacgttttggaacgtggtgtcggctatgttctgagcgacgaggcgaccggtaagtatttcacggtgaccaaagaaggcaatttcattccgctgcaactgacgggtaaagagaaagttatcacgggtttctccagcgatggtaagggtatcacctatttcggtacgagcggtacgcaggcgaagtctgcgtttgttaccttcaatggtaacacctactatttcgacgcgcgtggccacatggttaccaatagcgaatacagcccgaatggcaaggacgtctaccgttttctgccgaacggtatcatgctgagcaatgcgttttacattgatgcgaacggtaatacctacctgtacaactctaagggtcaaatgtacaaaggcggttacacgaaattcgatgtttctgaaacggataaggacggtaaagagtccaaggtcgtcaagttccgctactttacgaacgaaggcgtcatggccaagggtgttaccgtcattgatggttttacccaatacttcggtgaggacggctttcaagcgaaggataagctggtcaccttcaagggcaagacgtattacttcgacgcacacactggtaatggtatcaaagatacctggcgcaatatcaatggtaaatggtactatttcgacgcgaatggcgttgctgcgaccggtgcgcaggtgattaacggccagaaactgtacttcaacgaggatggctcccaagtcaaaggcggcgtggttaagaacgcagacggcacctatagcaaatacaaagaaggttttggtgagctggttactaacgagtttttcacgactgatggcaatgtttggtactacgccggtgcaaatggtaaaaccgttaccggtgcacaagtgatcaacggccaacatttgtacttcaatgcggacggttcccaggtgaagggtggcgttgtcaagaacgcggatggcacctacagcaagtacaatgctagcactggtgaacgtctgacgaacgagttctttacgaccggtgataacaattggtattacattggcgcaaacggtaagagcgtgacgggtgaggtcaagattggtgatgatacttactttttcgcgaaggatggcaaacaagttaaaggtcaaaccgtcagcgccggtaatggtcgcattagctactactacggtgacagcggcaagcgtgcggttagcacctggattgagattcagccgggtgtttatgtgtatttcgacaaaaacggtttggcgtaccctccgcgtgttctgaattaa

SEQ ID NO: 8 - protein, 1477 aa, Streptococcus salivarius
MDETQDKTVTQSNSGTTASLVTSPEATKEADKRTNTKEADVLTPAKETNAVETATTTNTQATAEAATTATTADVAVAAVPNKEAVVTTDAPAVTTEKAEEQPATVKAEVVNTEVKAPEAALKDSEVEAALSLKNIKNIDGKYYYVNEDGSHKENFAITVNGQLLYFGKDGALTSSSTYSFTPGTTNIVDGFSINNRAYDSSEASFELIDGYLTADSWYRPASIIKDGVTWQASTAEDFRPLLMAWWPNVDTQVNYLNYMSKVFNLDAKYSSTDKQETLKVAAKDIQIKIEQKIQAEKSTQWLRETISAFVKTQPQWNKETENYSKGGGEDHLQGGALLYVNDSRTPWANSDYRRLNRTATNQTGTIDKSILDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSLNDNHYNDKTDGAALAMENKQRLALLFSLAKPIKERTPAVSPLYNNTFNTTQRDEKTDWINKDGSKAYNEDGTVKQSTIGKYNEKYGDASGNYVFIRAHDNNVQDIIAEIIKKEINPKSDGFTITDAEMKQAFEIYNKDMLSSDKKYTLNNIPAAYAVMLQNMETITRVYYGDLYTDDGHYMETKSPYYDTIVNLMKSRIKYVSGGQAQRSYWLPTDGKMDNSDVELYRTNEVYTSVRYGKDIMTANDTEGSKYSRTSGQVTLVANNPKLNLDQSAKLNVEMGKIHANQKYRALIVGTADGIKNFTSDADAIAAGYVKETDSNGVLTFGANDIKGYETFDMSGFVAVWVPVGASDNQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANSKSSGKDYQAKYGGEFLAELKAKYPEMFKVNMISTGKPIDDSVKLKQWKAEYFNGTNVLERGVGYVLSDEATGKYFTVTKEGNFIPLQLTGKEKVITGFSSDGKGITYFGTSGTQAKSAFVTFNGNTYYFDARGHMVTNSEYSPNGKDVYRFLPNGIMLSNAFYIDANGNTYLYNSKGQMYKGGYTKFDVSETDKDGKESKVVKFRYFTNEGVMAKGVTVIDGFTQYFGEDGFQAKDKLVTFKGKTYYFDAHTGNGIKDTWRNINGKWYYFDANGVAATGAQVINGQKLYFNEDGSQVKGGVVKNADGTYSKYKEGFGELVTNEFFTTDGNVWYYAGANGKTVTGAQVINGQHLYFNADGSQVKGGVVKNADGTYSKYNASTGERLTNEFFTTGDNNWYYIGANGKSVTGEVKIGDDTYFFAKDGKQVKGQTVSAGNGRISYYYGDSGKRAVSTWIEIQPGVYVYFDKNGLAYPPRVLN

SEQ ID NO: 9 - DNA, 4311 bp, Streptococcus downei
  source 1..4311: mol_type = unassigned DNA
atggttgacggcaaatactactactacgatcaggacggcaacgtaaagaaaaacttcgcggttagcgtgggcgagaaaatctattactttgacgaaactggcgcctacaaagacaccagcaaagttgaggcggacaaaagcggcagcgacattagcaaggaagagactaccttcgcggcaaacaaccgcgcctacagcaccagcgcggagaattttgaggcgatcgacaattatctgaccgcggactcctggtatcgtcctaaatccatcctgaaggatggcaaaacgtggacggaaagcagcaaagatgactttcgtccgctgctgatggcgtggtggccggataccgaaacgaagcgcaattacgtgaactacatgaacaaagttgttggcatcgacaagacctataccgcggaaaccagccaggccgacttgaccgctgcggcggaactggtgcaagcacgcattgagcagaagatcacgaccgaacagaacacgaaatggctgcgtgaggcaatctcggcatttgttaaaacgcaaccgcagtggaacggtgaaagcgagaagccgtacgacgatcacctgcaaaacggtgctctgaaatttgataatcagagcgacctgaccccggatacgcaaagcaactaccgtctgttgaaccgtaccccgactaatcagacgggtagcctggacagccgcttcacttataacgcgaacgaccctttgggcggttatgagctgctgctggcaaatgacgtcgataacagcaatccgatcgtgcaggcggagcagctgaactggctgcattacctgctgaattttggtacgatctacgccaaagatgccgacgctaacttcgatagcattcgtgtggacgcggttgataacgtcgatgcggatctgctgcaaattagcagcgattacctgaaagcagcctacggcattgataagaataacaaaaacgcgaacaaccacgtgagcattgtcgaagcctggagcgataatgataccccgtacctgcatgacgatggtgacaacctgatgaatatggataacaaatttcgcctgtccatgctgtggtcgctggccaaaccgctggacaagcgtagcggtctgaacccgctgattcataacagcttggtggatcgtgaagttgatgaccgcgaggttgaaacggttccgagctattcttttgcacgtgcgcatgatagcgaggtccaggacttgatccgtgacatcatcaaggcagagatcaatccgaacgcattcggttatagctttacccaagacgagattgaccaggcctttaagatttacaatgaggatctgaagaaaacggataagaaatacacccactataatgtgccgttgagctacaccctgctgctgacgaataagggtagcatcccacgtgtctactatggtgatatgtttaccgacgatggtcagtatatggcgaacaaaaccgtcaactatgacgccattgaatctctgctgaaagcgcgtatgaagtatgtcgctggcggtcaagcaatgcagaactaccaaatcggtaatggtgagatcctgaccagcgttcgttatggtaagggtgccctgaaacagagcgacaaaggtgatgcgaccacgcgcaccagcggtgtcggtgtcgttatgggcaatcagccaaactttagcttggacggcaaagtggtggctctgaacatgggcgcagctcatgcgaatcaggagtatcgtgcgctgatggttagcacgaaagacggtgttgccacgtatgcgaccgatgcagatgcgagcaaagccggtctggtcaaacgtaccgacgaaaacggctacctgtatttcctgaatgacgacctgaagggtgtggccaatcctcaggtgagcggtttcttgcaggtgtgggttccggtgggtgccgcggatgatcaagatatccgtgttgcagctagcgataccgcatccaccgatggcaagagcctgcaccaagacgccgcgatggatagccgtgttatgtttgaaggcttctctaactttcagtcctttgccacgaaagaagaggaatataccaacgtcgttatcgccaacaatgtggataagttcgttagctggggtatcacggatttcgagatggccccacaatatgtttccagcaccgacggtcaattcctggactctgtcattcagaacggttatgcttttacggaccgttatgacttgggcatgtctaaggcaaacaaatacggcacggccgatcaactggttaaggccattaaggccctgcacgcgaagggcctgaaggttatggcagattgggtgccggatcagatgtataccttcccgaaacaggaagtcgtgaccgttacccgtaccgacaaatttggcaaaccgatcgcaggttcccaaatcaatcatagcctgtatgttaccgataccaagtccagcggcgatgactatcaggccaaatatggtggtgcgtttctggacgagctgaaggagaaatatccggagctgttcacgaagaaacaaatcagcacgggtcaagctattgacccgagcgtgaaaatcaaacagtggtctgctaagtatttcaatggctccaacatcctgggtcgcggtgcggactacgtactgtcggatcaggcgagcaacaaatacctgaacgtgtctgacgataaactgttcctgccgaaaaccttgctgggccaagttgtcgagagcggtatccgctttgacggcactggttatgtgtacaactctagcactacgggtgaaaaagttaccgattccttcattacggaggcaggtaatctgtactacttcggtcaagacggctatatggtgaccggcgcacagaacattaagggcagcaactattacttcctggccaatggtgcggccctgcgtaacaccgtttacaccgatgcgcaaggtcagaatcactattacggcaacgacggcaagcgttatgagaatggttaccaacagttcggcaacgattcttggcgttacttcaaaaatggcgtgatggcgctgggtctgactacggtggatggtcacgtgcagtatttcgataaagatggtgtccaggccaaggataagatcattgtcacccgcgatggcaaagtccgctatttcgaccagcacaacggtaatgcggttactaacacgttcgttgcggacaagacgggtcactggtactatctgggcaaagacggcgtcgcggttaccggtgcgcagactgtgggtaaacagcatttgtactttgaagcgaacggtcaacaagtcaagggtgacttcgtgacggctaaagacggtaaactgtacttctatgatgtggacagcggcgacatgtggaccaatacctttatcgaggataaagcgggtaattggttctacctgggtaaggacggtgcggccgtcaccggtgcacagacgatcaaaggccagaaattgtatttcaaagccaacggtcagcaagttaaaggtgacattgtcaaggacgcggacggtaagatccgttattacgacgctcagaccggtgaacaggtctttaacaagtccgttagcgtcaacggtaagacctactatttcggtagcgacggcaccgcgcaaacccaggcgaatccgaaaggccaaacctttaaggatggtagcggcgttctgcgtttctacaatttggagggccagtatgtctcgggcagcggctggtacgaaacggccgagcacgagtgggtatatgtgaaatccggtaaagttctgaccggtgcccagacgattggtaatcaacgtgtttacttcaaggacaatggtcaccaggtgaaaggccagctggtcacgggtaatgacggtaaattgcgttactacgacgcgaacagcggtgatcaagcattcaacaaatccgtcacggttaacggtaaaacctactactttggcagcgatggtacggcgcagacgcaggctaatcctaagggtcagaccttcaaagatggtagcggcgtgctgcgtttttacaacttggaaggccaatacgtgtctggcagcggttggtacaagaatgcgcagggccagtggctgtacgtgaaagatggcaaggtcctgaccggtctgcaaacggtcggcaatcagaaggtctacttcgacaaaaatggcatccaagcaaagggtaaggccgttcgcacgtccgatggtaaagtgcgctactttgatgagaatagcggtagcatgattacgaaccaatggaagttcgtttacggtcaatactattacttcggttctgacggcgcagcggtttaccgtggttggaactaa

SEQ ID NO: 10 - protein, 1436 aa, Streptococcus downei
MVDGKYYYYDQDGNVKKNFAVSVGEKIYYFDETGAYKDTSKVEADKSGSDISKEETTFAANNRAYSTSAENFEAIDNYLTADSWYRPKSILKDGKTWTESSKDDFRPLLMAWWPDTETKRNYVNYMNKVVGIDKTYTAETSQADLTAAAELVQARIEQKITTEQNTKWLREAISAFVKTQPQWNGESEKPYDDHLQNGALKFDNQSDLTPDTQSNYRLLNRTPTNQTGSLDSRFTYNANDPLGGYELLLANDVDNSNPIVQAEQLNWLHYLLNFGTIYAKDADANFDSIRVDAVDNVDADLLQISSDYLKAAYGIDKNNKNANNHVSIVEAWSDNDTPYLHDDGDNLMNMDNKFRLSMLWSLAKPLDKRSGLNPLIHNSLVDREVDDREVETVPSYSFARAHDSEVQDLIRDIIKAEINPNAFGYSFTQDEIDQAFKIYNEDLKKTDKKYTHYNVPLSYTLLLTNKGSIPRVYYGDMFTDDGQYMANKTVNYDAIESLLKARMKYVAGGQAMQNYQIGNGEILTSVRYGKGALKQSDKGDATTRTSGVGVVMGNQPNFSLDGKVVALNMGAAHANQEYRALMVSTKDGVATYATDADASKAGLVKRTDENGYLYFLNDDLKGVANPQVSGFLQVWVPVGAADDQDIRVAASDTASTDGKSLHQDAAMDSRVMFEGFSNFQSFATKEEEYTNVVIANNVDKFVSWGITDFEMAPQYVSSTDGQFLDSVIQNGYAFTDRYDLGMSKANKYGTADQLVKAIKALHAKGLKVMADWVPDQMYTFPKQEVVTVTRTDKFGKPIAGSQINHSLYVTDTKSSGDDYQAKYGGAFLDELKEKYPELFTKKQISTGQAIDPSVKIKQWSAKYFNGSNILGRGADYVLSDQASNKYLNVSDDKLFLPKTLLGQVVESGIRFDGTGYVYNSSTTGEKVTDSFITEAGNLYYFGQDGYMVTGAQNIKGSNYYFLANGAALRNTVYTDAQGQNHYYGNDGKRYENGYQQFGNDSWRYFKNGVMALGLTTVDGHVQYFDKDGVQAKDKIIVTRDGKVRYFDQHNGNAVTNTFVADKTGHWYYLGKDGVAVTGAQTVGKQHLYFEANGQQVKGDFVTAKDGKLYFYDVDSGDMWTNTFIEDKAGNWFYLGKDGAAVTGAQTIKGQKLYFKANGQQVKGDIVKDADGKIRYYDAQTGEQVFNKSVSVNGKTYYFGSDGTAQTQANPKGQTFKDGSGVLRFYNLEGQYVSGSGWYETAEHEWVYVKSGKVLTGAQTIGNQRVYFKDNGHQVKGQLVTGNDGKLRYYDANSGDQAFNKSVTVNGKTYYFGSDGTAQTQANPKGQTFKDGSGVLRFYNLEGQYVSGSGWYKNAQGQWLYVKDGKVLTGLQTVGNQKVYFDKNGIQAKGKAVRTSDGKVRYFDENSGSMITNQWKFVYGQYYYFGSDGAAVYRGWN

SEQ ID NO: 11 - DNA, 3942 bp, Streptococcus mutans
  source 1..3942: mol_type = unassigned DNA
atgattgacggcaaatactactactatgacaacaacggcaaagtacgcaccaatttcacgttgatcgcggacggtaaaatcctgcattttgatgaaactggcgcgtacaccgacactagcattgataccgtgaacaaggatattgtcacgacgcgtagcaacctgtataagaaatacaatcaagtgtatgatcgcagcgcgcagagcttcgagcatgttgatcactacctgacggcggaatcttggtaccgtccgaaatacattctgaaagatggcaagacctggacccagagcaccgagaaggacttccgtcctctgctgatgacctggtggccgagccaggaaacgcagcgccagtatgtcaacttcatgaacgcccagttgggtatcaacaaaacgtacgacgacaccagcaatcagctgcaattgaacatcgctgctgcaacgatccaagcaaagatcgaagccaaaatcacgacgctgaagaacaccgattggctgcgtcaaacgatcagcgcgttcgtcaaaacccaaagcgcttggaatagcgacagcgaaaagccgtttgatgaccatctgcaaaacggtgcggttctgtatgataacgaaggtaaattgacgccgtatgccaatagcaactatcgtattctgaaccgcacgccgaccaaccagaccggtaagaaggacccgcgttataccgccgacaacacgatcggcggctacgagtttctgctggccaacgacgtggataatagcaacccggtggttcaggccgagcagctgaactggctgcacttcctgatgaactttggtaatatctacgcaaacgaccctgacgctaacttcgactccatccgcgttgacgctgtcgataatgtggacgccgatctgttacagatcgcgggtgactatctgaaagcggcaaagggcatccataagaatgacaaagcggcgaacgaccacctgtccattctggaagcgtggagcgacaatgacactccgtatctgcatgatgatggcgacaacatgattaacatggataacaaactgcgcctgagcctgctgttctccctggcgaaaccgctgaatcagcgtagcggtatgaacccgttgattacgaacagcctggtcaaccgtactgatgataatgccgaaacggcggcagtgccaagctactcttttatccgtgcccacgatagcgaggtccaggatttgattcgtgatatcattaaggctgagattaacccgaacgtcgtcggttacagcttcacgatggaagagattaagaaggcatttgagatctacaataaggacctgttggccacggagaagaagtatacccactataacaccgcattgagctacgcgttgctgctgacgaacaagagcagcgtgccgcgtgtctactatggtgatatgtttacggacgatggtcaatacatggcccacaagaccattaactacgaggcaatcgaaaccctgctgaaagcacgtatcaagtacgtgtccggtggtcaggctatgcgcaaccagcaagtgggtaattcggagatcatcaccagcgtgcgttacggtaaaggtgcgctgaaggcgatggatacgggtgaccgcactacccgtacctctggtgtggcggtcattgagggcaacaacccgagcttgcgcctgaaggcttctgatcgtgtggttgtgaatatgggtgcggcccacaaaaatcaagcctatcgcccgctgctgttgacgaccgataacggcattaaggcctatcacagcgaccaagaagcggcaggcctggtgcgttacaccaacgaccgtggcgaactgatctttaccgcagccgacattaagggctacgcaaatccgcaagttagcggctacctgggcgtctgggtccctgttggcgcagcagctgatcaggacgttcgtgttgcggcgagcaccgcgccaagcacggacggcaagagcgttcaccagaacgcggctctggacagccgtgtgatgttcgagggtttctcgaacttccaggcatttgctaccaagaaagaagagtataccaatgtggtcatcgctaagaatgtggataagttcgcggagtggggtgtcaccgatttcgagatggctccgcaatacgtttctagcaccgacggtagctttttggatagcgtgattcaaaacggttatgcttttaccgaccgttacgacctgggcatcagcaagccgaacaaatatggcaccgcggacgatctggttaaagcgattaaggcattgcacagcaaaggcatcaaagttatggcggattgggttccggaccagatgtatgccctgccggaaaaagaggttgtgacggcaacccgtgttgacaaatacggtacgccggtagctggcagccagatcaaaaacacgctgtacgtggtcgatggtaaatctagcggtaaggaccagcaggcgaagtacggtggtgccttcctggaagagctgcaagcgaagtatccggaactgttcgcgcgcaaacagattagcaccggtgttccgatggacccgagcgtcaagattaagcaatggagcgcaaaatacttcaacggcacgaatatcctgggtcgtggtgctggttacgtgctgaaagatcaggcaaccaacacctactttaacatcagcgacaataaagagatcaatttcctgccaaagacgttgctgaaccaggattctcaagttggctttagctacgacggtaagggctatgtgtactacagcacctcgggctaccaggctaaaaacacgttcatcagcgagggtgacaagtggtattacttcgacaataacggttatatggttaccggcgcacagagcattaatggtgtgaactattacttcctgccgaatggtttacagctgcgtgatgcgattctgaaaaatgaggacggtacgtacgcgtattatggcaatgatggtcgccgctacgagaatggctattatcagtttatgagcggtgtttggcgccatttcaataatggcgagatgtccgttggtctgaccgtcattgacggtcaagttcaatactttgacgagatgggttaccaggcgaaaggcaaattcgttaccaccgcggatggtaagatccgttacttcgataagcagagcggcaatatgtatcgtaatcgtttcattgagaacgaagagggcaaatggctgtacctgggtgaggacggcgcggcagtcaccggtagccagacgatcaatggtcagcacctgtattttcgtgctaacggcgttcaggttaagggtgagttcgtgaccgatcgtcatggccgcatctcttattacgacggcaacagcggtgatcagatccgcaaccgtttcgtccgcaatgcgcaaggccagtggttttactttgacaacaatggctatgcagtaactggtgctcgtacgatcaacggccagcacctgtatttccgcgcgaacggtgttcaggtaaaaggtgagtttgttacggaccgccacggccgcattagctattatgatggtaatagcggtgaccaaattcgcaatcgtttcgtgcgtaatgcacagggtcagtggttctacttcgacaataatggttatgcagtcacgggtgcacgtaccattaacggccaacacctgtactttcgcgccaatggtgtgcaagtgaaaggcgaatttgttactgatcgttatggtcgtatcagctactatgatggcaattctggcgaccaaattcgcaatcgctttgttcgtaacgcccaaggtcaatggttctatttcgacaacaacggttacgcggtgaccggtgcccgcacgattaatggtcaacacttgtacttccgtgccaacggtgtccaggtgaagggtgaatttgtgaccgaccgctatggtcgcatttcttactacgacgcaaattccggtgaacgcgtccgtatcaattaa

SEQ ID NO: 12 - protein, 1313 aa, Streptococcus mutans
MIDGKYYYYDNNGKVRTNFTLIADGKILHFDETGAYTDTSIDTVNKDIVTTRSNLYKKYNQVYDRSAQSFEHVDHYLTAESWYRPKYILKDGKTWTQSTEKDFRPLLMTWWPSQETQRQYVNFMNAQLGINKTYDDTSNQLQLNIAAATIQAKIEAKITTLKNTDWLRQTISAFVKTQSAWNSDSEKPFDDHLQNGAVLYDNEGKLTPYANSNYRILNRTPTNQTGKKDPRYTADNTIGGYEFLLANDVDNSNPVVQAEQLNWLHFLMNFGNIYANDPDANFDSIRVDAVDNVDADLLQIAGDYLKAAKGIHKNDKAANDHLSILEAWSDNDTPYLHDDGDNMINMDNKLRLSLLFSLAKPLNQRSGMNPLITNSLVNRTDDNAETAAVPSYSFIRAHDSEVQDLIRDIIKAEINPNVVGYSFTMEEIKKAFEIYNKDLLATEKKYTHYNTALSYALLLTNKSSVPRVYYGDMFTDDGQYMAHKTINYEAIETLLKARIKYVSGGQAMRNQQVGNSEIITSVRYGKGALKAMDTGDRTTRTSGVAVIEGNNPSLRLKASDRVVVNMGAAHKNQAYRPLLLTTDNGIKAYHSDQEAAGLVRYTNDRGELIFTAADIKGYANPQVSGYLGVWVPVGAAADQDVRVAASTAPSTDGKSVHQNAALDSRVMFEGFSNFQAFATKKEEYTNVVIAKNVDKFAEWGVTDFEMAPQYVSSTDGSFLDSVIQNGYAFTDRYDLGISKPNKYGTADDLVKAIKALHSKGIKVMADWVPDQMYALPEKEVVTATRVDKYGTPVAGSQIKNTLYVVDGKSSGKDQQAKYGGAFLEELQAKYPELFARKQISTGVPMDPSVKIKQWSAKYFNGTNILGRGAGYVLKDQATNTYFNISDNKEINFLPKTLLNQDSQVGFSYDGKGYVYYSTSGYQAKNTFISEGDKWYYFDNNGYMVTGAQSINGVNYYFLPNGLQLRDAILKNEDGTYAYYGNDGRRYENGYYQFMSGVWRHFNNGEMSVGLTVIDGQVQYFDEMGYQAKGKFVTTADGKIRYFDKQSGNMYRNRFIENEEGKWLYLGEDGAAVTGSQTINGQHLYFRANGVQVKGEFVTDRHGRISYYDGNSGDQIRNRFVRNAQGQWFYFDNNGYAVTGARTINGQHLYFRANGVQVKGEFVTDRHGRISYYDGNSGDQIRNRFVRNAQGQWFYFDNNGYAVTGARTINGQHLYFRANGVQVKGEFVTDRYGRISYYDGNSGDQIRNRFVRNAQGQWFYFDNNGYAVTGARTINGQHLYFRANGVQVKGEFVTDRYGRISYYDANSGERVRIN

SEQ ID NO: 13 - DNA, 3972 bp, Streptococcus dentirousetti
  source 1..3972: mol_type = unassigned DNA
atggttgacggcaaatactactactacgatgcagacggcaacgtaaagaaaaacttcgcggttagcgttggcgatgccattttctattttgatgaaacgggtgcctacaaagataccagcaaagttgatgcggataagaccagctctagcgtcaatcagaccacggaaacgttcgcagcgaataaccgtgcgtatagcaccgcagccgagaactttgaagcgattgataactacctgactgcggatagctggtatcgtccgaagtctatcttgaaagatggtacgacgtggaccgaaagcaccaaggatgattttcgcccgctgctgatggcgtggtggccggataccgaaaccaaacgtaactacgtgaactatatgaacaaggtggtcggtatcgacaaaacgtacaccgcggaaacgtcccaagctgacctgacggcggcagccgaactggtgcaggcgcgtatcgagcagaaaatcactagcgaaaagaatacgaagtggctgcgtgaggcgatttccgcgttcgttaagactcaaccgcagtggaatggcgagagcgagaaaccttatgatgaccacctgcaaaatggtgcgctgaagttcgacaatgaaaccagcctgaccccggatacgcagagcggctatcgcatcctgaaccgtaccccgacgaatcaaaccggtagcctggacccgcgcttcacctttaatcagaatgacccgctgggtggttatgagtatttgctggctaatgatgtcgataacagcaacccggtcgttcaggccgagagcctgaactggctgcattacctgctgaattttggtagcatttacgcgaatgatccggaggccaatttcgacagcatccgtgtggacgcggtggacaatgttgacgcagacctgctgcaaattagctcggattacctgaaatcggcgtacaaaattgacaagaacaacaaaaatgcgaacgaccacgttagcatcgtcgaggcgtggagcgacaatgataccccgtacctgaatgatgatggcgacaatctgatgaacatggataacaagtttcgtctgagcatgctgtggagcctggcgaagccaaccaatgtccgtagcggcttgaatccgctgatccacaacagcgtggttgaccgtgaggtggacgaccgtgaagttgaggctaccccgaattacagctttgcacgcgcacacgacagcgaagttcaagatttgattcgcgacatcatcaaagctgagatcaacccaaacagcttcggttatagctttacccaagaggaaatcgaccaggccttcaagatctacaatgaggatttgaagaaaaccaataagaagtatacccactacaacgtcccgctgagctacaccctgctgctgacgaacaagggcagcattccacgcatttactacggtgacatgtttacggatgacggtcagtatatggccaacaaaaccgttaactatgacgccattgagagcctgctgaaagcacgtatgaagtatgttagcggtggccaagcgatgcagaattacaacatcggcaacggcgagattctgaccagcgtccgttacggtaagggtgccctgaaacagagcgacaaaggcgataagactactcgtaccagcggtattggcgttgtgatgggtaaccagagcaatttcagcctggagggcaaggtggtggccctgaatatgggtgcaacgcataccaaacagaagtatcgtgcattgatggtgtctacggaaaccggcgtggcgatttacaatagcgatgaagaagcagaggcagcaggcctgatcaaaacgaccgatgagaatggttatttgtactttctgaatgacgatctgaagggcgtggctaacccgcaggtcagcggcttcctgcaagtgtgggttccggttggtgcaccggctgaccaggacattcgtgtggcggcgaccgatgcggcttctaccgacggtaagagcctgcatcaggacgcagctctggattctcgcgtcatgtttgaaggtttcagcaacttccagagcttcgcaaccaaggaagaggaatacaccaacgttgttattgcaaagaacgtggataagttcgtgagctggggtatcaccgacttcgagatggcaccgcagtacgttagctctaccgatggcacctttctggatagcgtgattcaaaatggctatgcctttacggaccgttacgacctgggtatgagcaaagcaaacaagtatggtactgctgaccaactggtggccgcgattaaagcgctgcatgcgaagggtctgcgtgtgatggcggattgggtcccagatcaaatgtacactttccctaagaaggaagtggttaccgttacccgtacggacaaatttggcaatccagtggcaggcagccaaatcaaccacaccttgtacgtcactgatactaagggtagcggtgacgactaccaggcgaagtacggtggcgcattcctggatgaactgaaagaaaagtacccggagctgtttaccaagaagcaaatcagcaccggtcaggcaatcgacccgagcgtgaaaatcaagcagtggagcgcgaagtacttcaacggtagcaatatcttgggtcgcggtgcgaactacgtgctgtccgaccaggcgtctaacaagtactttaacgtggccgaaggtaaagtctttctgccagcggcgatgctgggtaaggtcgtcgagagcggtatccgtttcgacggtaaaggttatatctataacagcagcaccactggcgaacaagtgaaggacagcttcattaccgaagcgggtaacttgtactattttggcaaagatggttatatggtcatgggtgcacagaatatccagggtgctaactactacttcttggcgaatggtgcggccctgcgcaatagcatcctgacggatcaggatggcaaaagccactattatgcaaatgacggcaagcgttatgagaacggctactatcaattcggtaacgactcctggcgctattttgaaaacggcgttatggccgttggtttgacgcgcgttgcgggccacgaccaatactttgataaggatggtatccaagcgaagaataagatcattgttacgcgtgacggtaaggtccgctacttcgacgaacacaacggcaatgctgccacgaatacgtttatcagcgatcaagccggccattggtactacctgggtaaagatggtgtcgccgtgacgggtgcgcagaccgttggcaagcaacacctgtacttcgaggctaacggccaacaagtaaaaggcgattttgttaccgccaaggacggtaagttgtattttctggacggtgactctggcgacatgtggaccgataccttcgtccaggataaggctggtcattggttctatctgggcaaagacggtgcggcggtaaccggtgcccagaccgtccgtggtcagaagctgtacttcaaagcgaatggccagcaggttaagggtgacattgtgaaaggcgcggatggtaaaatccgttactatgatgcaaattccggtgaccaggtttacaatcgcacggtgaaaggctccgacggcaagacctatatcattggtaatgacggcgtcgcaatcacgcaaaccatcgccaaaggccagaccatcaaggatggcagcgttctgcgcttctatagcatggagggtcagctggtgaccggcagcggctggtattccaacgcgaaaggtcaatggttgtatgtcaagaacggtcaagtcctgacgggtttgcagacggtgggcagccagcgtgtgtactttgacgcaaatggtattcaagcgaaaggtaaagcagtgcgtacctccgatggcaaactgcgttacttcgatgcgaacagcggcagcatgatcaccaatcagtggaaagaagttaatggtcagtactactatttcgacaacaacggtgttgcgatctatcgcggttggaactaa

SEQ ID NO: 14 - protein, 1323 aa, Streptococcus dentirousetti
MVDGKYYYYDADGNVKKNFAVSVGDAIFYFDETGAYKDTSKVDADKTSSSVNQTTETFAANNRAYSTAAENFEAIDNYLTADSWYRPKSILKDGTTWTESTKDDFRPLLMAWWPDTETKRNYVNYMNKVVGIDKTYTAETSQADLTAAAELVQARIEQKITSEKNTKWLREAISAFVKTQPQWNGESEKPYDDHLQNGALKFDNETSLTPDTQSGYRILNRTPTNQTGSLDPRFTFNQNDPLGGYEYLLANDVDNSNPVVQAESLNWLHYLLNFGSIYANDPEANFDSIRVDAVDNVDADLLQISSDYLKSAYKIDKNNKNANDHVSIVEAWSDNDTPYLNDDGDNLMNMDNKFRLSMLWSLAKPTNVRSGLNPLIHNSVVDREVDDREVEATPNYSFARAHDSEVQDLIRDIIKAEINPNSFGYSFTQEEIDQAFKIYNEDLKKTNKKYTHYNVPLSYTLLLTNKGSIPRIYYGDMFTDDGQYMANKTVNYDAIESLLKARMKYVSGGQAMQNYNIGNGEILTSVRYGKGALKQSDKGDKTTRTSGIGVVMGNQSNFSLEGKVVALNMGATHTKQKYRALMVSTETGVAIYNSDEEAEAAGLIKTTDENGYLYFLNDDLKGVANPQVSGFLQVWVPVGAPADQDIRVAATDAASTDGKSLHQDAALDSRVMFEGFSNFQSFATKEEEYTNVVIAKNVDKFVSWGITDFEMAPQYVSSTDGTFLDSVIQNGYAFTDRYDLGMSKANKYGTADQLVAAIKALHAKGLRVMADWVPDQMYTFPKKEVVTVTRTDKFGNPVAGSQINHTLYVTDTKGSGDDYQAKYGGAFLDELKEKYPELFTKKQISTGQAIDPSVKIKQWSAKYFNGSNILGRGANYVLSDQASNKYFNVAEGKVFLPAAMLGKVVESGIRFDGKGYIYNSSTTGEQVKDSFITEAGNLYYFGKDGYMVMGAQNIQGANYYFLANGAALRNSILTDQDGKSHYYANDGKRYENGYYQFGNDSWRYFENGVMAVGLTRVAGHDQYFDKDGIQAKNKIIVTRDGKVRYFDEHNGNAATNTFISDQAGHWYYLGKDGVAVTGAQTVGKQHLYFEANGQQVKGDFVTAKDGKLYFLDGDSGDMWTDTFVQDKAGHWFYLGKDGAAVTGAQTVRGQKLYFKANGQQVKGDIVKGADGKIRYYDANSGDQVYNRTVKGSDGKTYIIGNDGVAITQTIAKGQTIKDGSVLRFYSMEGQLVTGSGWYSNAKGQWLYVKNGQVLTGLQTVGSQRVYFDANGIQAKGKAVRTSDGKLRYFDANSGSMITNQWKEVNGQYYYFDNNGVAIYRGWN

SEQ ID NO: 15 - DNA, 4047 bp, Streptococcus oralis
  source 1..4047: mol_type = unassigned DNA
atgatcgacggcaaaaactactacgtacaggatgatggcacggtaaagaagaatttcgcggtagaactgaatggtcgtatcctgtattttgatgcagaaaccggcgctctggttgatagcaacgagtatcagttccaacagggtacgagcagcctgaacaatgaattttctcagaagaacgcattctatggtacgaccgataaggatattgagactgtggatggctacctgaccgcagatagctggtatcgcccgaaattcatcctgaaggatggcaagacgtggaccgcgagcacggaaacggatctgcgtccgctgttgatggcatggtggccggacaagcgtacccaaatcaactatctgaactacatgaaccagcagggtctgggtgcgggtgcgtttgagaacaaagtggagcaggccctgctgacgggtgcaagccaacaggtacaacgcaagatcgaagagaagattggtaaagagggtgataccaagtggctgcgcaccctgatgggtgcgttcgtgaaaacgcaaccaaactggaatatcaaaaccgagtctgaaacgaccggcacgaaaaaggaccatctgcaaggcggtgcactgctgtatacgaacaacgagaaatccccgcacgcggacagcaaatttcgtctgctgaatcgtaccccgaccagccaaaccggcacgccgaagtatttcatcgacaagtctaacggtggctacgaatttctgctggcgaacgattttgacaatagcaatcctgcggtacaagctgagcagctgaattggctgcactacatgatgaactttggcagcattgttgcgaatgatccgaccgcgaatttcgacggcgttcgtgtggatgctgttgataacgtcaatgcggacttgttgcaaattgcaagcgattactttaagagccgttacaaagtcggtgagagcgaagaagaagcgatcaagcacctgtccatcctggaagcatggagcgataacgacccggactacaacaaagataccaagggtgcacagttggcgattgataacaaactgcgcctgagcctgctgtactctttcatgcgtaatctgagcatccgtagcggtgttgaaccgacgattaccaatagcctgaatgaccgttccagcgaaaagaagaacggcgagcgtatggcaaattacatcttcgtgcgtgcccacgatagcgaggtccaaacggtgatcgccgacatcattcgcgaaaacatcaatccgaacaccgacggcctgacgtttacgatggacgagctgaagcaggcattcaagatttacaacgaggacatgcgcaaggcggacaaaaagtatacccagtttaacattcctaccgcacacgcgctgatgctgtctaataaggattctattacccgcgtgtactatggtgatctgtatactgacgatggtcagtacatggagaagaaaagcccgtatcacgatgcgattgacgctctgctgcgtgcacgtattaaatacgtcgcgggtggccaggatatgaaagtgacctatatgggcgtgccgcgtgaagcggataagtggagctataacggcattctgaccagcgtgcgctatggcacgggcgctaacgaagccacggatgagggcactgcggaaacgcgcacgcaaggtatggcagtgattgcgagcaataatccaaatctgaaactgaatgaatgggacaagttgcaagtcaacatgggtgcggcgcataagaatcaatattaccgtccggttctgctgaccactaaggacggtatcagccgttatctgaccgatgaagaagtgcctcagagcctgtggaaaaagacggacgcaaacggtattctgaccttcgacatgaatgatattgctggctacagcaacgtgcaagttagcggttacctggccgtctgggtcccggtcggtgcgaaggcggatcaagatgcgcgcacgaccgcatccaagaagaaaaatgcgtcgggtcaggtgtacgaaagcagcgcggctctggatagccagctgatttacgaaggtttcagcaactttcaagactttgccactcgcgatgatcagtacacgaacaaggtcattgcgaaaaacgtgaatctgttcaaagaatggggtgtgaccagcttcgagctgccgccgcagtacgtgagcagccaagatggcacctttctggacagcattatccaaaacggctatgcatttgaagaccgttacgatatggcgatgagcaagaataacaagtatggtagcctgaaagacctgttgaacgcgctgcgcgcactgcacagcgtcaacattcaagcaatcgccgattgggtgccggaccaaatttacaacttgccgggcaaagaggtggtgaccgcaactcgtgtcaacaactacggcacctaccgtgagggtgctgaaatcaaagaaaagctgtatgtcgccaatagcaagaccaacgaaaccgatttccaaggtaaatacggtggtgcgttcctggatgagctgaaggcgaagtacccggagattttcgagcgtgtccaaatcagcaacggccaaaagatgactaccgatgaaaagatcaccaaatggagcgcgaaatactttaatggcaccaatattctgggtcgtggcgcgtactatgtcctgaaagattgggccagcaatgattacctgacgaaccgtaacggcgagattgttttgccgaagcaactggttaacaagaatagctataccggctttgtcagcgacgcgaacggcacgaagttctattctacctctggctaccaggcgaagaacagcttcattcaagacgaaaacggtaattggtattactttgacaaacgtggttatctggttacgggcgcacacgagattgatggcaagcatgtctacttcctgaaaaacggtatccaactgcgtgacagcatccgtgaggatgagaacggtaatcaatactattacgaccagaccggcgcacaagtgctgaaccgttactacacgacggacggtcagaattggcgctatttcgatgcgaaaggtgttatggcacgcggcctggtaaagattggtgacggccaacagtttttcgatgaaaacggttaccaggtcaagggcaagattgttagcgcaaaagacggcaagctgcgctactttgataaagactctggcaatgctgtcattaatcgtttcgcgcagggtgacaatccgagcgactggtactatttcggtgtggaatttgctaaactgacgggtttgcaaaagatcggccagcagacgctgtattttgaccaagacggtaagcaagtcaaaggtaagatcgtaactctgtcggacaaaagcattcgttacttcgatgccaacagcggtgaaatggcggttggcaagttcgcggaaggtgcaaagaatgagtggtattatttcgataaaaccggcaaagcggttactggtttgcagaaaattggtaagcagaccctgtactttgaccaggacggtaaacaggttaaaggcaaggttgtcacgctggctgataaaagcatccgctacttcgacgcagactccggcgagatggcggtcggtaagtttgcagagggtgcgaagaacgagtggtactattttgatcagactggcaaggccgtgactggtttgcaaaagattgacaagcaaaccttgtacttcgaccaggacggtaaacaagtcaagggtaagattgtgacgttgagcgacaagtcgatccgttactttgatgctaatagcggtgagatggctactaacaaattcgtcgagggctcgcagaatgaatggtactacttcgatcaagcgggtaaggctgttacgggcttgcaacaggtcggtcagcaaactctgtacttcacccaggatggtaagcaagtgaagggtaaggtcgtggacgtgaacggtgtttctcgttatttcgacgcaaactccggtgacatggctcgttctaaatggattcaactggaagatggcagctggatgtatttcgaccgtgacggtcgtggccagaattttggccgtaactaa

SEQ ID NO: 16 - protein, 1348 aa, Streptococcus oralis
MIDGKNYYVQDDGTVKKNFAVELNGRILYFDAETGALVDSNEYQFQQGTSSLNNEFSQKNAFYGTTDKDIETVDGYLTADSWYRPKFILKDGKTWTASTETDLRPLLMAWWPDKRTQINYLNYMNQQGLGAGAFENKVEQALLTGASQQVQRKIEEKIGKEGDTKWLRTLMGAFVKTQPNWNIKTESETTGTKKDHLQGGALLYTNNEKSPHADSKFRLLNRTPTSQTGTPKYFIDKSNGGYEFLLANDFDNSNPAVQAEQLNWLHYMMNFGSIVANDPTANFDGVRVDAVDNVNADLLQIASDYFKSRYKVGESEEEAIKHLSILEAWSDNDPDYNKDTKGAQLAIDNKLRLSLLYSFMRNLSIRSGVEPTITNSLNDRSSEKKNGERMANYIFVRAHDSEVQTVIADIIRENINPNTDGLTFTMDELKQAFKIYNEDMRKADKKYTQFNIPTAHALMLSNKDSITRVYYGDLYTDDGQYMEKKSPYHDAIDALLRARIKYVAGGQDMKVTYMGVPREADKWSYNGILTSVRYGTGANEATDEGTAETRTQGMAVIASNNPNLKLNEWDKLQVNMGAAHKNQYYRPVLLTTKDGISRYLTDEEVPQSLWKKTDANGILTFDMNDIAGYSNVQVSGYLAVWVPVGAKADQDARTTASKKKNASGQVYESSAALDSQLIYEGFSNFQDFATRDDQYTNKVIAKNVNLFKEWGVTSFELPPQYVSSQDGTFLDSIIQNGYAFEDRYDMAMSKNNKYGSLKDLLNALRALHSVNIQAIADWVPDQIYNLPGKEVVTATRVNNYGTYREGAEIKEKLYVANSKTNETDFQGKYGGAFLDELKAKYPEIFERVQISNGQKMTTDEKITKWSAKYFNGTNILGRGAYYVLKDWASNDYLTNRNGEIVLPKQLVNKNSYTGFVSDANGTKFYSTSGYQAKNSFIQDENGNWYYFDKRGYLVTGAHEIDGKHVYFLKNGIQLRDSIREDENGNQYYYDQTGAQVLNRYYTTDGQNWRYFDAKGVMARGLVKIGDGQQFFDENGYQVKGKIVSAKDGKLRYFDKDSGNAVINRFAQGDNPSDWYYFGVEFAKLTGLQKIGQQTLYFDQDGKQVKGKIVTLSDKSIRYFDANSGEMAVGKFAEGAKNEWYYFDKTGKAVTGLQKIGKQTLYFDQDGKQVKGKVVTLADKSIRYFDADSGEMAVGKFAEGAKNEWYYFDQTGKAVTGLQKIDKQTLYFDQDGKQVKGKIVTLSDKSIRYFDANSGEMATNKFVEGSQNEWYYFDQAGKAVTGLQQVGQQTLYFTQDGKQVKGKVVDVNGVSRYFDANSGDMARSKWIQLEDGSWMYFDRDGRGQNFGRN

SEQ ID NO: 17 - DNA, 4047 bp, Streptococcus sanguinis
  source 1..4047: mol_type = unassigned DNA
atgattgatggtaaaaagtattacgtacaggacgacggcacggttaagaagaatttcgcggttgagctgaatggcaagatcctgtacttcgatgcagagactggtgcgttgattgacagcgcggagtatcaattccaacaaggcaccagcagcctgaataatgagttcactcaaaagaacgccttttacggtacgaccgataaggatgtggaaaccattgatggttacttgaccgccgattcctggtatcgtccgaagttcattctgaaagatggcaaaacctggacggcgagcacggaaattgacttgcgtccgttgttgatggcgtggtggccggacaaacagacccaggttagctacctgaattacatgaaccagcaaggcttgggtgcaggcgccttcgaaaacaaagtagagcaggcaattctgaccggtgcgtcccaacaggtacaacgtaaaatcgaagaacgcatcggtaaagagggtgataccaagtggctgcgtaccctgatgggtgcatttgtaaagacccagccgaactggaacattaagaccgagtccgaaaccactggcacgaataaagatcatctgcaaggtggcgcactgctgtatagcaattccgacaagacgagccatgccaactctaagtaccgtatcctgaaccgcaccccgaccaaccaaacgggcacgccgaaatactttattgacaagagcaatggtggttatgaatttctgctggcgaatgactttgacaatagcaatccggcagtgcaagcggaacagctgaactggttgcactttatgatgaattttggctccatcgttgcaaatgatccgacggccaacttcgacggcgtccgcgttgacgctgtggataacgtgaatgcggatctgttgcaaattgcgagcgactatttcaagagccgctataaagtcggcgaaagcgaagaagaggccattaagcacctgtccatcctggaagcgtggagcgacaacgacccggactacaacaaggatactaaaggtgcccaactgccgatcgacaacaaactgcgtctgagcctgctgtactccttcatgcgtaagctgagcatccgtagcggcgtcgagccgaccatcaccaactctctgaatgatcgcagcacggagaagaagaatggtgagcgtatggcaaactatatcttcgttcgtgcacatgatagcgaggtgcaaacggtcatcgccgacattatccgtgagaacatcaatccgaataccgacggcctgacgttcacgatggatgaactgaagcaggcctttaaaatttacaatgaggatatgcgtaaagccgacaaaaagtacacgcagttcaatatcccgaccgcgcacgcgctgatgctgagcaacaaagattctatcacccgcgtttactacggtgacctgtatacggatgacggtcagtatatggaaaagaaaagcccgtatcacgacgccattgacgctctgctgcgtgcgcgtatcaaatatgttgcgggtggtcaggacatgaaggtgacctatatgggcgtgccgcgtgaggcagataaatggagctataacggcatcctgaccagcgttcgttatggtacgggtgccaacgaggcaaccgacgagggtacggcagaaacccgtacccagggcatggccgtcattgccagcaacaatccgaacctgaaactgaacgagtgggacaagttgcaggtcaacatgggtgcagctcacaaaaaccaatactatcgtccggtgctgctgaccaccaaggacggcatctcgcgctacctgaccgacgaagaagtcccgcagagcctgtggaaaaagaccgatgcgaacggcatcttgacgtttgacatgaatgatattgcgggttacagcaacgtccaagtgagcggttatctggccgtctgggttcctgtgggtgcgaaggcggaccaggacgctcgtgttacggcatctaagaagaaaaatgcctctggccaagtttacgaaagcagcgcagccctggactcccagctgatctatgagggcttcagcaattttcaggactttgccacccgtgacgaccagtacactaacaaggttatcgcgaaaaacgtcaatctgtttaaagagtggggcgtcaccagcttcgaattgccgccacagtatgtgagcagccaagacggtacgttcctggatagcatcatccagaatggttatgcattcgaagatcgctatgatatggcgatgagcaaaaacaataagtacggtagcttgaacgacctgttgaacgccttgcgtgcactgcatagcgtgaatatccaagcgattgcggattgggtgccggaccagatttacaatctgccgggtaaagaagttgtcactgcaacccgtgttaacaattatggcacgtatcgtgagggtagcgagattaaagagaacctgtacgttgctaacaccaaaaccaatggtacggactaccaaggtaagtatggtggtgcgttcttggacgagctgaaagccaaataccctgagatttttgagcgcgtccaaatcagcaacggccagaagatgaccaccgacgagaagattacgaaatggtccgccaaacactttaacggcacgaacattctgggtcgtggtgcgtattatgtgctgaaagactgggcgagcaacgagtacctgaataacaaaaatggcgagatggttctgccgaagcagctggttaataaaaatgcatataccggcttcgtcagcgacgcgagcggcaccaaatactattctaccagcggctatcaggctcgtaatagctttattcaagatgaaaatggtaattggtactacttcaataaccgtggttatttggtgacgggtgcacaggaaatcgacggtaagcaactgtatttcctgaaaaacggcattcagctgcgtgattctctgcgtgaggacgaaaacggcaaccagtattactatgataagacgggtgcgcaagttctgaatcgttattacactacggacggccaaaattggcgctacttcgacgttaaaggcgtcatggcccgtggtctggtcacgatgggtggtaaccaacaattctttgaccaaaacggttaccaggttaaaggcaaaattgcgcgtgcaaaagacggtaaactgcgttacttcgataaagacagcggtaatgcggcagctaaccgtttcgcccaaggcgataaccctagcgactggtactatttcggtgcagatggtgttgcggttacgggcctgcaaaaggttggtcagcaaactctgtactttgatcaggacggcaagcaggtgaaaggtaaagttgttaccttggcggacaaaagcattcgttatttcgatgcaaacagcggcgagatggcggtgaacaagtttgtggaaggtgctaagaacgtgtggtactacttcgatcaagcaggcaaagcggtgaccggcctgcaaaccatcaataaacaagtgctgtatttcgaccaggatggtaaacaagtcaaaggtaaggtggtcacgctggctgataagtctatccgctacttcgacgcgaacagcggtgagatggcagtgggcaaattcgccgaaggcgcaaagaatgagtggtattactttgaccaggcgggcaaggctgttaccggtctgcaaaagatcggccaacagacgctgtatttcgaccagaacggtaaacaggttaagggtaaagtggtcaccctggcggataagagcatccgctatttcgacgctaactctggcgaaatggcaagcaataagttcgttgagggtgccaaaaatgaatggtactatttcgatcaggctggcaaggcagtgacgggtctgcaacaaattggccagcagaccctgtattttgaccagaatggcaaacaggtgaagggtaagattgtgtatgttaatggtgcgaatcgctactttgatgccaatagcggtgaaatggcgcgtaacaagtggattcagctggaagatggcagctggatgtattttgaccgcaatggtcgtggtcgtcgtttcggttggaactaa

SEQ ID NO: 18 - protein, 1348 aa, Streptococcus sanguinis
MIDGKKYYVQDDGTVKKNFAVELNGKILYFDAETGALIDSAEYQFQQGTSSLNNEFTQKNAFYGTTDKDVETIDGYLTADSWYRPKFILKDGKTWTASTEIDLRPLLMAWWPDKQTQVSYLNYMNQQGLGAGAFENKVEQAILTGASQQVQRKIEERIGKEGDTKWLRTLMGAFVKTQPNWNIKTESETTGTNKDHLQGGALLYSNSDKTSHANSKYRILNRTPTNQTGTPKYFIDKSNGGYEFLLANDFDNSNPAVQAEQLNWLHFMMNFGSIVANDPTANFDGVRVDAVDNVNADLLQIASDYFKSRYKVGESEEEAIKHLSILEAWSDNDPDYNKDTKGAQLPIDNKLRLSLLYSFMRKLSIRSGVEPTITNSLNDRSTEKKNGERMANYIFVRAHDSEVQTVIADIIRENINPNTDGLTFTMDELKQAFKIYNEDMRKADKKYTQFNIPTAHALMLSNKDSITRVYYGDLYTDDGQYMEKKSPYHDAIDALLRARIKYVAGGQDMKVTYMGVPREADKWSYNGILTSVRYGTGANEATDEGTAETRTQGMAVIASNNPNLKLNEWDKLQVNMGAAHKNQYYRPVLLTTKDGISRYLTDEEVPQSLWKKTDANGILTFDMNDIAGYSNVQVSGYLAVWVPVGAKADQDARVTASKKKNASGQVYESSAALDSQLIYEGFSNFQDFATRDDQYTNKVIAKNVNLFKEWGVTSFELPPQYVSSQDGTFLDSIIQNGYAFEDRYDMAMSKNNKYGSLNDLLNALRALHSVNIQAIADWVPDQIYNLPGKEVVTATRVNNYGTYREGSEIKENLYVANTKTNGTDYQGKYGGAFLDELKAKYPEIFERVQISNGQKMTTDEKITKWSAKHFNGTNILGRGAYYVLKDWASNEYLNNKNGEMVLPKQLVNKNAYTGFVSDASGTKYYSTSGYQARNSFIQDENGNWYYFNNRGYLVTGAQEIDGKQLYFLKNGIQLRDSLREDENGNQYYYDKTGAQVLNRYYTTDGQNWRYFDVKGVMARGLVTMGGNQQFFDQNGYQVKGKIARAKDGKLRYFDKDSGNAAANRFAQGDNPSDWYYFGADGVAVTGLQKVGQQTLYFDQDGKQVKGKVVTLADKSIRYFDANSGEMAVNKFVEGAKNVWYYFDQAGKAVTGLQTINKQVLYFDQDGKQVKGKVVTLADKSIRYFDANSGEMAVGKFAEGAKNEWYYFDQAGKAVTGLQKIGQQTLYFDQNGKQVKGKVVTLADKSIRYFDANSGEMASNKFVEGAKNEWYYFDQAGKAVTGLQQIGQQTLYFDQNGKQVKGKIVYVNGANRYFDANSGEMARNKWIQLEDGSWMYFDRNGRGRRFGWN

SEQ ID NO: 19 - DNA, 4023 bp, unidentified
  misc_feature 1..4023: note = unknown Streptococcus sp. C150
  source 1..4023: mol_type = unassigned DNA
atgatcgacggcaaatactactacgtaaacgaggacggcagccacaaagagaatttcgcgatcacggttaatggtcaactgctgtattttggtaaggatggcgcgctgaccagcagcagcacgtacagcttcacccaaggcactaccaatattgtggacggttttagcattaacaaccgtgcgtatgactccagcgaggcctctttcgagctgattgacggttatctgactgcggactcttggtaccgtccggcgagcattatcaaagacggtgtgacgtggcaagcatccaccgccgaggacttccgcccgttgctgatggcgtggtggccgaacgttgatactcaggtgaactacctgaactacatgtccaaagtctttaatctggatgctaaatacagctcgactgataaacaggaaaccctgaaggtggcggcgaaagatatccagatcaaaattgaacaaaagattcaggcggaaaagtccacgcaatggctgcgtgaaacgatcagcgcctttgtaaaaacccagccgcaatggaacaaagagactgagaactacagcaagggcggtggtgaggaccatctgcaaggtggtgccctgctgtatgttaatgactctcgtaccccgtgggcgaacagcaactatcgtttgctgaaccgcacggcgaccaaccagaccggtacgatcgacaagagcatcctggacgagcagagcgatccgaatcacatgggtggttttgatttcttgctggctaatgacgttgacttgagcaatccggtcgtccaggcggaacaactgaatcagatccactacctgatgaattggggttctattgtcatgggtgataaagacgcgaattttgacggtattcgtgtagacgcggtggataatgttgatgcggacatgctgcaattgtacaccaactatttccgcgaatactatggtgtcaacaaaagcgaggcaaacgcgctggcgcacattagcgtcctggaagcctggagcctgaatgacaaccattacaatgataagactgatgttgcggcgctggcaatggagaataagcagcgcttggcactgttgtttagcctggcgaaaccgattaaagaacgcacgcctgccgtgtctccgctgtacaacaatacgtttaacaccactcagcgtgatgaaaagacggactggatcaataaagatggttcgaaagcctacaatgaggatggcactgtcaagaaaagcaccatcggcaagtataacgagaagtatggtgatgctagcggcaactacgttttcatccgcgctcacgacaataacgtgcaagacatcatcgcggagatcattaagaaagagattaacgagaaatctgacggttttaccattacggattcggagatgaagcgtgcatttgagatctataacaaagacatgctgtctaatgacaaaaagtacacgctgaataacatcccggcggcgtacgcggttatgctgcaaaacatggaaacgattacccgcgtgtattacggcgatctgtacacggacgacggtaattacatggaagcgaaaagcccgtactacgatacgattgttaacttgatgaagtctcgcatcaaatacgtgagcggtggccaggcgcagcgcagctactggctgccgaccgatggtaagatggataagtcggatgttgagctgtaccgtacgaacgaagtgtacacgagcgtccgttacggcaaagacattatgaccgccgatgacacgcaaggtagcaaatacagccgtaccagcggtcaggtgaccctggtcgtcaacaacccaaaactgaccttggaccaaagcgcaaagctgaacgtggttatgggcaagattcatgctaatcagaagtaccgcgcactgattgtcggtaccccgaacggtattaagaatttcaccagcgacgcagaggctattgccgcaggctatgtcaaagaaaccgatggcaatggcgtgctgaccttcggtgcaaacgacatcaagggttatgaaactttcgatatgagcggcttcgtcgctgtttgggttccggtcggtgcgagcgacgaccaagatattcgtgtggcggcgtctacggcagcaaagaaagagggtgagctgacgctgaaagcgaccgaagcctatgactcccaactgatctatgaaggctttagcaatttccagaccatcccagatggcagcgatccttctgtttataccaatcgtaagatcgcggaaaatgttgatttgttcaagagctggggtgtcacgagcttcgaaatggctccgcagttcgtttctgcggacgatggcacgtttctggacagcgtcattcaaaacggctatgcgttcgcagaccgttatgatctggccatgagcaaaaacaataagtacggtagcaaagaagatctgcgtaacgcgctgaaggcactgcacaaagcaggcattcaggcgattgcagattgggtgccagaccaaatctaccagctgcctggcaaagaagttgttactgccacccgcacggacggtgctggtcgcaaaatcagcgatgcaatcatcgatcattccctgtacgttgcgaactccaagagctccggtaaggactaccaagcgaagtacggtggcgagttcttggcggaactgaaggcgaaatacccggaaatgttcaaagtgaacatgattagcaccggcaaaccgattgatgatagcgtgaaactgaagcagtggaaagcagaatacttcaacggcaccaatgtgctggatcgcggtgtcggttatgttctgagcgatgaggcaaccggtaagtatttcaccgttaccaaagagggtaactttatcccgttgcagctgaagggtaacaagaaggtgattaccggcttttccagcgacggtaagggcattacctatttcggtactagcggtaaccaagctaaatccgcgttcgtcacttttaacggtaacacgtactacttcgacgcacgtggccacatggttaccaacggtgagtactcgccgaatggtaaagatgtgtatcgttttctgccgaacggcattatgctgagcaacgcgttctatgttgacggcaatggcaacacctacctgtacaactccaaaggccaaatgtataaaggtggctatagcaaatttgacgtcacggaaacgaaggacggtaaagagagcaaagttgtcaagttccgctactttacgaacgagggcgtgatggcgaaaggtgtcacggttgtggatggcttcactcagtactttaacgaggatggcattcaaagcaaagacgagctggtcacttacaatggcaagacctattacttcgaagcacacacgggcaatgccattaagaatacgtggcgtaatatcaagggcaaatggtaccattttgatgctaacggtgtcgcggctactggcgcacaggttatcaacggtcagcacctgtacttcaatgaagatggctctcaagtaaaaggtagcatcgtcaaaaacgctgatggtacgttcagcaagtacaaggacagctctggcgatctggtggtgaacgagtttttcacgacgggtgataacgtctggtactatgctggtgccaatggcaaaacggttactggtgcacaggtgattaatggccagcacttgttcttcaaagaggatggcagccaggtcaagggcgactttgtgaagaatagcgacggcacctactccaagtatgacgctgcgagcggcgaacgtctgaccaacgagttcttcactacgggcgacaatcattggtactatattggcgccaacggtaagaccgttaccggtgaagttaagattggtgacgacacgtatttcttcgcaaaagacggtaagcaactgaaaggtcaaatcgttaccacccgtagcggtcgtatcagctactactttggtgatagcggtaagaaggctattagcacgtgggtggagatccagccgggtgtgtttgttttcttcgacaaaaacggcctggcttacccaccggagaatatgaactga

SEQ ID NO: 20 - protein, 1340 aa, unidentified
  REGION 1..1340: note = unknown Streptococcus sp. C150
MIDGKYYYVNEDGSHKENFAITVNGQLLYFGKDGALTSSSTYSFTQGTTNIVDGFSINNRAYDSSEASFELIDGYLTADSWYRPASIIKDGVTWQASTAEDFRPLLMAWWPNVDTQVNYLNYMSKVFNLDAKYSSTDKQETLKVAAKDIQIKIEQKIQAEKSTQWLRETISAFVKTQPQWNKETENYSKGGGEDHLQGGALLYVNDSRTPWANSNYRLLNRTATNQTGTIDKSILDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSLNDNHYNDKTDVAALAMENKQRLALLFSLAKPIKERTPAVSPLYNNTFNTTQRDEKTDWINKDGSKAYNEDGTVKKSTIGKYNEKYGDASGNYVFIRAHDNNVQDIIAEIIKKEINEKSDGFTITDSEMKRAFEIYNKDMLSNDKKYTLNNIPAAYAVMLQNMETITRVYYGDLYTDDGNYMEAKSPYYDTIVNLMKSRIKYVSGGQAQRSYWLPTDGKMDKSDVELYRTNEVYTSVRYGKDIMTADDTQGSKYSRTSGQVTLVVNNPKLTLDQSAKLNVVMGKIHANQKYRALIVGTPNGIKNFTSDAEAIAAGYVKETDGNGVLTFGANDIKGYETFDMSGFVAVWVPVGASDDQDIRVAASTAAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRNALKALHKAGIQAIADWVPDQIYQLPGKEVVTATRTDGAGRKISDAIIDHSLYVANSKSSGKDYQAKYGGEFLAELKAKYPEMFKVNMISTGKPIDDSVKLKQWKAEYFNGTNVLDRGVGYVLSDEATGKYFTVTKEGNFIPLQLKGNKKVITGFSSDGKGITYFGTSGNQAKSAFVTFNGNTYYFDARGHMVTNGEYSPNGKDVYRFLPNGIMLSNAFYVDGNGNTYLYNSKGQMYKGGYSKFDVTETKDGKESKVVKFRYFTNEGVMAKGVTVVDGFTQYFNEDGIQSKDELVTYNGKTYYFEAHTGNAIKNTWRNIKGKWYHFDANGVAATGAQVINGQHLYFNEDGSQVKGSIVKNADGTFSKYKDSSGDLVVNEFFTTGDNVWYYAGANGKTVTGAQVINGQHLFFKEDGSQVKGDFVKNSDGTYSKYDAASGERLTNEFFTTGDNHWYYIGANGKTVTGEVKIGDDTYFFAKDGKQLKGQIVTTRSGRISYYFGDSGKKAISTWVEIQPGVFVFFDKNGLAYPPENMN

SEQ ID NO: 21-24 - skipped (4 entries)

SEQ ID NO: 25 - DNA, 4308 bp, Streptococcus sobrinus
  source 1..4308: mol_type = unassigned DNA
atggttgacggcaaatactactattatgatcaggatggcaacgttaagaagaatttcgcggttagcgttggtgacaagatctactactttgacgagactggtgcctacaaagacacctctaaagtggacgcggacaagtctagcagcgccgttagccaaaatgcgacgatctttgcggctaacaatcgtgcgtatagcacctctgctgagaactttgaggccgttgataactatctgacggcagatagctggtatcgtcctaaatctattctgaaagatggcaagacgtggaccgagtcgggtaaggacgacttccgtccgctgctgatggcgtggtggccggacacggagactaaacgcaattacgtgaattacatgaacctggttgtcggcatcgacaagacgtacaccgcggaaacctctcaagcagatttgaccgcagcggcggagctggtccaggcgcgtattgaacagaaaatcaccacggaacagaatacgaaatggctgcgcgaggcgatctctgctttcgtcaagacccagccgcagtggaatggtgaaagcgagaagccgtatgacgaccacctgcaaaacggtgctctgaaattcgataatcagagcgacctgaccccggacacccagagcaactatcgcctgctgaatcgcaccccgactaaccagactggcagcctggacagccgtttcacctataatgcgaacgatccgttgggtggctacgaatttctgctggctaacgacgtggataatagcaaccctgtggtgcaggcagaacaactgaactggttgcattacctgttgaattttggtagcatttacgcgaaagatgcggatgcaaacttcgattccatccgtgtggacgccgtggacaacgtcgatgcagatctgttgcagattagcagcgattacctgaaggcagcctatggcattgacaagaacaataagaacgcgaacaaccatgttagcattgttgaggcttggagcgataacgatacgccgtacctgcacgatgacggtgataacctgatgaacatggacaataagttccgcttgagcatgctgtggagcctggccaagccgctggacaagcgcagcggtctgaatcctctgattcataacagcctggtggaccgtgaggttgatgaccgtgaagtggaaacggttccgagctactcttttgcgcgtgcgcatgattccgaggtccaagacattatccgcgacattatcaaggccgaaatcaacccgaatagctttggttatagcttcacccaagaagagattgaccaggcgtttaagatctataatgaagatctgaagaaaaccgacaagaaatacacccactataatgtcccgttgagctatactttgctgctgacgaataaaggttcgattccgcgtgtgtattacggtgatatgttcaccgatgatggtcaatacatggcgaacaaaacggttaactatgatgccattgagtcgctgctgaaagcgcgcatgaagtacgttagcggcggtcaagcgatgcaaaactatcaaatcggcaatggtgagattctgaccagcgttcgttatggtaagggtgcattgaagcaatccgacaagggtgacgcgaccacgcgtacgtccggtgtgggcgtcgtgatgggcaaccagccgaactttagcctggacggcaaggtggtggcattgaacatgggtgccgctcatgcaaatcaggagtatcgtgcgctgatggtgagcaccaaggatggcgttgccacgtatgccaccgacgcggacgcaagcaaggcaggtctggtcaaacgcaccgatgaaaatggttatttgtactttctgaacgacgatctgaagggtgtggcaaacccacaagtcagcggtttcttgcaggtgtgggtcccagtgggtgcggctgacgatcaggacattcgtgttgcagcgagcgacacggctagcacggacggtaagtccctgcatcaagatgcggcaatggatagccgtgttatgtttgagggttttagcaacttccagagctttgcaaccaaagaagaagagtacaccaacgtagttattgcgaacaacgtggacaaattcgttagctggggtattaccgactttgagatggcaccgcaatatgtcagctccaccgatggccagtttctggatagcgttatccagaatggttacgcgttcaccgaccgttatgatctgggtatgagcaaagccaacaaatacggtaccgcggatcagctggttaaagcaatcaaagcgttgcacgcgaagggtctgaaggtgatggcggactgggttccagaccagatgtacacgtttccgaagcaggaagttgtcactgtcacgcgcaccgacaaatttggtaagccgattgcgggcagccaaatcaatcacagcctgtacgtgacggacaccaaatccagcggtgatgattaccaggccaaatatggtggtgcgttcctggatgagctgaaagagaaatacccggagctgttcaccaaaaagcagatctcgaccggtcaggcgatcgacccgagcgtgaagattaagcagtggagcgcgaaatactttaatggtagcaacattctgggtcgtggtgccgactacgtcctgtccgatcaagttagcaacaagtatttcaatgtggccagcgacacgctgtttctgccgtctagcctgttgggtaaggttgtcgaaagcggtattcgttacgatggcaaaggttatatctataacagcagcgcgactggcgaccaagtcaaggcgtcttttatcacggaagcaggcaatctgtactacttcggcaaagacggttacatggttactggtgcgcagaccattaacggtgcgaattacttcttcttggaaaatggtacggccctgcgtaataccatctacaccgatgcacagggcaactcccactattatgctaatgatggcaagcgttacgagaacggttaccagcagttcggcaacgattggcgttacttcaaagatggtaacatggccgtcggtctgaccacggtggatggtaacgttcagtatttcgacaaggacggtgtccaagctaaagacaagattattgtgacccgcgatggtaaggtgcgctactttgatcaacacaatggcaacgcggtcacgaatacctttatcgccgacaagaccggtcactggtactacctgggcaaagatggcgtcgcggtcaccggcgctcaaaccgtcggtaagcaaaaactgtattttgaggcgaacggtgagcaggtgaaaggcgactttgtgactagccatgaaggcaaactgtacttttatgatgttgacagcggcgacatgtggaccgataccttcatcgaggataaggccggcaactggttctacctgggtaaagacggcgcagcagttagcggtgcacagaccattcgcggtcaaaagctgtacttcaaggcgtacggtcaacaggtcaaaggtgacatcgttaaaggcaccgacggcaagatccgttactacgatgcgaaatccggcgagcaggttttcaataagacggtcaaagccgctgatggcaaaacctatgtgatcggcaacaatggtgtggcggtcgatccgagcgttgttaagggtcagacgttcaaagacgccagcggcgcactgcgtttttacaatctgaaaggtcaactggttacgggctccggttggtatgaaacggccaatcacgattgggtgtatattcagagcggtaaagcactgaccggtgagcaaaccatcaatggtcagcacctgtactttaaagaagatggccaccaagttaaaggtcagctggtcacccgtacggacggcaaagtgcgttactatgacgcaaattctggcgatcaagcgttcaacaagtccgtgacggttaacggcaaaacgtattacttcggtaatgatggtaccgcgcaaaccgcgggtaacccgaaaggccaaatcttcaaggacggcagcgttctgcgtttctatagcatggaaggccagctggtaattggcagcggctggtattccaacgcgcaaggccaatggctgtatgtgaagaatggtaaagtgttgaccggtttgcagaccgtcggttcccagcgcgtgtactttgatgagaatggcattcaagcaaaaggcaaagcggttcgcacgagcgacggcaaaattcgctacttcgacgagaacagcggtagcatgatcaccaatcaatggaagtttgtttacggtcaatactattactttggtaatgacggtgcggcaatctaccgtggttggaattaa

SEQ ID NO: 26 - protein, 1435 aa, Streptococcus sobrinus
MVDGKYYYYDQDGNVKKNFAVSVGDKIYYFDETGAYKDTSKVDADKSSSAVSQNATIFAANNRAYSTSAENFEAVDNYLTADSWYRPKSILKDGKTWTESGKDDFRPLLMAWWPDTETKRNYVNYMNLVVGIDKTYTAETSQADLTAAAELVQARIEQKITTEQNTKWLREAISAFVKTQPQWNGESEKPYDDHLQNGALKFDNQSDLTPDTQSNYRLLNRTPTNQTGSLDSRFTYNANDPLGGYEFLLANDVDNSNPVVQAEQLNWLHYLLNFGSIYAKDADANFDSIRVDAVDNVDADLLQISSDYLKAAYGIDKNNKNANNHVSIVEAWSDNDTPYLHDDGDNLMNMDNKFRLSMLWSLAKPLDKRSGLNPLIHNSLVDREVDDREVETVPSYSFARAHDSEVQDIIRDIIKAEINPNSFGYSFTQEEIDQAFKIYNEDLKKTDKKYTHYNVPLSYTLLLTNKGSIPRVYYGDMFTDDGQYMANKTVNYDAIESLLKARMKYVSGGQAMQNYQIGNGEILTSVRYGKGALKQSDKGDATTRTSGVGVVMGNQPNFSLDGKVVALNMGAAHANQEYRALMVSTKDGVATYATDADASKAGLVKRTDENGYLYFLNDDLKGVANPQVSGFLQVWVPVGAADDQDIRVAASDTASTDGKSLHQDAAMDSRVMFEGFSNFQSFATKEEEYTNVVIANNVDKFVSWGITDFEMAPQYVSSTDGQFLDSVIQNGYAFTDRYDLGMSKANKYGTADQLVKAIKALHAKGLKVMADWVPDQMYTFPKQEVVTVTRTDKFGKPIAGSQINHSLYVTDTKSSGDDYQAKYGGAFLDELKEKYPELFTKKQISTGQAIDPSVKIKQWSAKYFNGSNILGRGADYVLSDQVSNKYFNVASDTLFLPSSLLGKVVESGIRYDGKGYIYNSSATGDQVKASFITEAGNLYYFGKDGYMVTGAQTINGANYFFLENGTALRNTIYTDAQGNSHYYANDGKRYENGYQQFGNDWRYFKDGNMAVGLTTVDGNVQYFDKDGVQAKDKIIVTRDGKVRYFDQHNGNAVTNTFIADKTGHWYYLGKDGVAVTGAQTVGKQKLYFEANGEQVKGDFVTSHEGKLYFYDVDSGDMWTDTFIEDKAGNWFYLGKDGAAVSGAQTIRGQKLYFKAYGQQVKGDIVKGTDGKIRYYDAKSGEQVFNKTVKAADGKTYVIGNNGVAVDPSVVKGQTFKDASGALRFYNLKGQLVTGSGWYETANHDWVYIQSGKALTGEQTINGQHLYFKEDGHQVKGQLVTRTDGKVRYYDANSGDQAFNKSVTVNGKTYYFGNDGTAQTAGNPKGQIFKDGSVLRFYSMEGQLVIGSGWYSNAQGQWLYVKNGKVLTGLQTVGSQRVYFDENGIQAKGKAVRTSDGKIRYFDENSGSMITNQWKFVYGQYYYFGNDGAAIYRGWN

SEQ ID NO: 27 - DNA, 4023 bp, Streptococcus salivarius
  source 1..4023: mol_type = unassigned DNA
atgattgacggcaaatactactacgtaaacaaagatggctcgcacaaagagaatttcgcaattaccgtgaatggtcagttgttgtatttcggtaaggacggtgcattgacgtctagcagcacctacagctttacgcagggcaccaccaacatcgttgatggctttagcaaaaacaaccgtgcgtacgattccagcgaggcgagctttgaactgatcgacggttatctgaccgcggactcctggtatcgtccggtgagcattatcaaggacggcgttacgtggcaagccagcaccaaagaggactttcgcccgctgctgatggcctggtggccgaatgttgacacccaggtcaactacctgaattacatgtcgaaggtgtttaacctggacgcgaagtatacgagcaccgacaaacaggttgacctgaatcgcgcagccaaggacattcaggttaagattgagcaaaagattcaggccgagaagagcactcaatggctgcgtgaagcgatttcggccttcgtcaaaacccagccgcagtggaataaagaaacggagaacttctccaagggtggtggtgaggatcatctgcaaggtggtgcactgctgtacgttaacgacccgcgtaccccgtgggctaactccaactaccgcctgctgaatcgtactgcgaccaaccagaccggcacgatcgacaagagcgttctggacgaacagagcgatcctaaccacatgggcggcttcgattttctgctggcgaatgacgtcgataccagcaatccggtggtgcaggcggaacaactgaatcagatccactacctgatgaattggggttccattgttatgggcgacaaagatgcaaacttcgatggtatccgcgtggacgcggtcgataacgttgacgcagatatgctgcaactgtacaccaactactttcgtgagtattatggcgtgaacaaaagcgaggcaaacgctttggcgcacatctcggtgctggaagcgtggagcttgaatgataatcactataatgacaagactgacggtgcggccctggcgatggagaacaaacagcgtttggccctgctgtttagcttggcgaaaccgatcaaagaacgtacccctgcggtgagcccgctgtacaacaacactttcaacacgacgcagcgtgacgaaaagaccgattggattaacaaagacggtagcaaagcctataatgaggacggcaccgtcaagcagtccaccatcggcaagtacaacgagaaatacggcgacgcgtccggcaattatgtgttcattcgcgcccacgataacaacgtccaagacattattgcagagatcattaagaaagaaatcaatccgaaaagcgacggtttcaccattaccgacgccgaaatgaaaaaggcattcgaaatctacaacaaagatatgctgtcctctgataagaaatacaccctgaacaacatcccagcggcctacgcggtgatgctgcaaaacatggaaaccattactcgtgtgtattacggcgatctgtataccgacgatggccattacatggaaaccaagagcccgtactacgacaccattgtgaacctgatgaagaaccgtatcaaatacgtgtccggtggtcaagcgcaacgttcctattggctgccgaccgacggtaagatggataaaagcgatgtcgaactgtatcgcaccaacgaggtgtacaccagcgtccgttacggtaaggacatcatgactgccgatgacacccaaggtagcaagtacagccgtaccagcggtcaggtgaccctggtggtgaacaacccgaagctgtctttggataagagcgcgaagctggacgtcgaaatgggcaagatccatgcaaaccagaaataccgtgctctgatcgtgggtacgccgaacggcatcaaaaacttcacgagcgacgccgaggcaatcgcggctggctacgtgaaagaaaccgacggcaatggtgtgctgaccttcggtgcaaatgacatcaaaggttacgaaacgtttgacatgagcggtttcgttgcagtttgggttccggtaggtgcaagcgatgatcaagacatccgtgtcgccgcaagcaccgcggcaaagaaagaaggtgagctgactttgaaggcaactgaggcgtatgactctcagctgatttacgaaggtttttcgaattttcagaccattccggatggtagcgatccgagcgtttacaccaatcgtaagatcgcggaaaatgttgatttgttcaagagctggggtgtgacctctttcgaaatggcgccacagtttgtgagcgcagacgacggtacgtttctggacagcgttatccagaacggctatgcgtttgcggaccgttatgatctggcgatgtccaaaaacaataagtacggttcgaaagaagatctgcgtaacgcgttgaaggctttgcacaaggccggcatccaagccattgcggactgggttccggatcagatctaccaactgccgggcaaagaagtagtgaccgccactcgtaccgatggtgccggtcgtaagattagcgatgcaattatcgatcacagcctgtacgtcgcaaacagcaagtcgtctggcaaagactatcaagctaaatacggtggtgagttcctggccgagctgaaagcaaagtacccggaaatgtttaaagtcaacatgattagcacgggtaaaccgatcgacgactctgtcaaactgaagcaatggaaggcggagtactttaacggtacgaatgttctggaccgtggtgttggttacgtcctgagcgatgaggcgacgggcaagtactttaccgttacgaaagagggtaactttatcccactgcaattgaaaggtaacgagaaagttatcacgggcttcagctctgacggcaagggcattacctatttcggcacctcgggtaatcaagcgaaaagcgcttttgtcacgttcaatggtaatacctactattttgacgcgcgtggccacatggttaccaacggcgaatatagccctaatggtaaggatgtgtatcgtttcctgccgaatggtattatgttgagcaatgcattctacgttgacggtaacggcaatacctacctgtacaactccaagggccaaatgtacaaaggtggttatagcaaattcgacgttacggaaaccaaagatggtaaagagagcaaagtggtgaaatttcgctactttaccaatgaaggtgtgatggcaaaaggtgttaccgtggtggacggcttcactcaatacttcaacgaagatggcattcagagcaaggacgaactggtgacctacaatggtaaaacctattacttcgaagcgcataccggtaatgcgatcaaaaacacgtggcgcaatatcaagggtaagtggtatcactttgatgcgaatggcgtggcggcaacgggtgcacaggttatcaatggtcagcacctgtactttaatgaggatggttcccaggtgaagggtggcgtcgtgaagaatgcggatggtaccttcagcaagtataaagatggttccggtgacctggtggtcaatgagttcttcactactggtgataacgtgtggtactacgctggtgccaacggcaaaactgtgacgggtgcccaggtcatcaatggccaacacctgtttttcaaagaggacggtagccaggttaagggtgatttcgttaagaacagcgacggcacctactctaagtatgatgcggccagcggcgaacgcctgacgaatgagtttttcacgaccggtgacaaccactggtactatattggtgccaatggcaaaaccgttaccggcgaagtcaagatcggtgatgatacgtacttcttcgcaaaagatggcaagcagctgaagggccagatcgtgacgacccgcagcggtcgtatcagctactacttcggcgactctggtaagaaggcgattagcacctgggtggagattcagccgggtgttttcgtgtttttcgacaaaaatggcctggcatatccgccggaaaacatgaattaa

SEQ ID NO: 28 - protein, 1340 aa, Streptococcus salivarius
MIDGKYYYVNKDGSHKENFAITVNGQLLYFGKDGALTSSSTYSFTQGTTNIVDGFSKNNRAYDSSEASFELIDGYLTADSWYRPVSIIKDGVTWQASTKEDFRPLLMAWWPNVDTQVNYLNYMSKVFNLDAKYTSTDKQVDLNRAAKDIQVKIEQKIQAEKSTQWLREAISAFVKTQPQWNKETENFSKGGGEDHLQGGALLYVNDPRTPWANSNYRLLNRTATNQTGTIDKSVLDEQSDPNHMGGFDFLLANDVDTSNPVVQAEQLNQIHYLMNWGSIVMGDKDANFDGIRVDAVDNVDADMLQLYTNYFREYYGVNKSEANALAHISVLEAWSLNDNHYNDKTDGAALAMENKQRLALLFSLAKPIKERTPAVSPLYNNTFNTTQRDEKTDWINKDGSKAYNEDGTVKQSTIGKYNEKYGDASGNYVFIRAHDNNVQDIIAEIIKKEINPKSDGFTITDAEMKKAFEIYNKDMLSSDKKYTLNNIPAAYAVMLQNMETITRVYYGDLYTDDGHYMETKSPYYDTIVNLMKNRIKYVSGGQAQRSYWLPTDGKMDKSDVELYRTNEVYTSVRYGKDIMTADDTQGSKYSRTSGQVTLVVNNPKLSLDKSAKLDVEMGKIHANQKYRALIVGTPNGIKNFTSDAEAIAAGYVKETDGNGVLTFGANDIKGYETFDMSGFVAVWVPVGASDDQDIRVAASTAAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRNALKALHKAGIQAIADWVPDQIYQLPGKEVVTATRTDGAGRKISDAIIDHSLYVANSKSSGKDYQAKYGGEFLAELKAKYPEMFKVNMISTGKPIDDSVKLKQWKAEYFNGTNVLDRGVGYVLSDEATGKYFTVTKEGNFIPLQLKGNEKVITGFSSDGKGITYFGTSGNQAKSAFVTFNGNTYYFDARGHMVTNGEYSPNGKDVYRFLPNGIMLSNAFYVDGNGNTYLYNSKGQMYKGGYSKFDVTETKDGKESKVVKFRYFTNEGVMAKGVTVVDGFTQYFNEDGIQSKDELVTYNGKTYYFEAHTGNAIKNTWRNIKGKWYHFDANGVAATGAQVINGQHLYFNEDGSQVKGGVVKNADGTFSKYKDGSGDLVVNEFFTTGDNVWYYAGANGKTVTGAQVINGQHLFFKEDGSQVKGDFVKNSDGTYSKYDAASGERLTNEFFTTGDNHWYYIGANGKTVTGEVKIGDDTYFFAKDGKQLKGQIVTTRSGRISYYFGDSGKKAISTWVEIQPGVFVFFDKNGLAYPPENMN

SEQ ID NO: 29 - DNA, 4026 bp, Streptococcus salivarius
  source 1..4026: mol_type = unassigned DNA
atgacggacggtaaatactattatgtaaatgaggacggcagccacaaagagaatttcgcaattacggtaaacggtcaactgttgtactttggcaaggacggcgctctgacgagcagcagcacgcacagcttcacgccgggtactacgaatattgtggacggtttctcgatcaacaaccgtgcgtacgatagcagcgaagcgagctttgagctgatcaacggttacctgacggcggattcctggtatcgcccggtttctatcatcaaggatggcgtcacgtggcaggcaagcactgccgaggattttcgtccgctgttgatggcctggtggccgaacgttgatacccaggtgaactatctgaactatatgtccaaggtctttaacctggaagccaagtacaccagcaccgataaacaggctgatctgaaccgtgctgcaaaggatatccaggtcaagatcgaacagaagatccaggcggaaaagagcacgcagtggctgcgtgagactatctccgcgtttgttaaaacccagccgcaatggaacaaagagactgagaattactccaagggtggtggcgaagatcatctgcaaggcggtgcgctgttgtacgtgaacgacagccgtaccccgtgggcgaatagcaattaccgcctgctgaatcgcacggcaacgaaccagaccggtaccattaacaagtcggtgttggacgagcaatccgatccaaatcacatgggtggcttcgacttcctgctggcaaacgatgtggatctgagcaatcctgttgtgcaggccgagcagctgaatcaaatccattatctgatgaactggggcagcattgttatgggtgacaaagacgcgaattttgatggtatccgtgtggacgccgttgacaacgtgaacgctgacatgttgcagctgtacacgaactactttcgtgagtattacggcgtcaacaaaagcgaagcgcaagcgctggcgcacattagcgttctggaagcgtggagcttgaacgataaccactataacgacaaaaccgatggtgcggcactggcgatggagaataagcaacgtctggccttgctgttctctctggccaagccgatcaaagatcgtactccggcagtgagcccactgtataacaatactttcaataccacccaacgtgacttcaagacggattggattaacaaggacggtagcaccgcctacaatgaggatggcaccgcgaaacaatctaccatcggtaagtacaatgagaaatatggtgatgcaagcggtaactatgtgtttattcgtgcccatgacaataacgtccaagacattattgcggagatcattaagaaagaaatcaataagaagagcgatggttttaccatcagcgatagcgaaatgaaacaggcgttcgaaatctacaacaaagatatgctgagcagcaataagaaatacactctgaataacattccggcagcgtacgccgtgatgctgcaaaacatggagactatcacccgtgtgtattatggtgacctgtacaccgacgacggtcactatatggaaaccaagagcccgtatcatgacaccattgtgaacctgatgaaaaaccgtatcaagtacgtttctggtggccaggcccaacgctcctattggctgccgaccgacggtaaaatggacaatagcgatgtcgaactgtaccgtactagcgaggtctataccagcgttcgctacggtaaggacattatgacggcggatgacaccgagggtagcaagtactcccgcacgagcggtcaggttaccctggttgttaacaacccgaagctgactctgcatgaaagcgccaaactgaacgtcgagatgggtaagatccacgcaaaccagaaataccgtgcgctgattgtgggtaccgccgatggcatcaaaaactttacgtctgatgccgaagcgatcgcggcaggctacgtaaaagaaacggacagcaatggtgttctgaccttcggcgcaaatgatatcaaaggttacgagactttcgatatgagcggtttcgtcgcagtttgggtgccggtgggtgcgagcgatgatcaggacatccgcgtggcgccgtcgacggaagcgaagaaagaaggtgaactgacgctgaaagccacggaagcgtatgatagccagttgatttatgaaggcttctccaatttccagaccattccggatggcagcgacccgagcgtttataccaaccgcaaaattgctgagaatgttgatctgtttaagtcctggggtgtcactagcttcgaaatggctccgcagtttgtttcggcggacgacggcaccttcctggatagcgttatccagaacggttacgcctttgcggaccgttatgatttggccatgagcaagaacaacaagtacggttctaaagaggatctgcgcgacgcactgaaagcgctgcacaaagctggcattcaggcaatcgcggactgggtcccagaccaaatctaccaactgccaggcaaagaagtggttacggcgacgcgcacggacggtgcgggtcgcaagatcgcggacgccatcattgatcatagcctgtatgttgctaactccaagagctccggtcgcgattaccaagcgcagtatggtggcgagtttctggcagagctgaaagcgaagtacccgaaaatgttcacggaaaacatgattagcacgggtaagccgatcgatgacagcgtcaaactgaagcaatggaaagccaagtatttcaatggtacgaatgtgctggaccgtggtgtcggttacgtcctgtccgacgaggcgaccggcaaatacttcaccgttaccaaagagggtaacttcattccgctgcaactgaccggcaatgaaaaagcggtgaccggtttcagcaacgacggcaagggtatcacctactttggtacgagcggtaatcaggccaagagcgcgttcgtcacctttaacggcaatacgtactatttcgacgcgcgtggccacatggtcacgaacggcgagtatagcccgaacggcaaagatgtctaccgttttctgccaaatggtattatgttgtcgaacgcgttttatgtcgacgcaaacggtaatacgtacttgtacaactacaagggccagatgtacaaaggtggttatacgaaatttgatgtcaccgaaactgataaagatggtaatgagagcaaggtggtcaagtttcgttatttcaccaatgagggcgtcatggctaagggtctgaccgtcattgacggtagcacccagtactttggtgaggatggttttcaaacgaaggacaagctggcgacctataaaggtaagacttattacttcgaggcacacacgggcaatgcgatcaaaaacacctggcgtaacatcgacggtaagtggtatcacttcgatgagaatggcgttgccgcgaccggtgcacaagtgattaacggtcaaaaactgtatttcaacgaggatggctcgcaagtgaagggcggtgttgttaagaacgccgacggtacctacagcaaatacaaagagggcagcggtgagctggttaccaacgagtttttcacgaccgacggtaatgtgtggtactatgctggtgcggatggcaagactgtgaccggtgctcaggtcattaatggtcagcacctgtactttaaagaagatggcagccaggtgaaaggtggtgtggtgaaaaacgcggacggtacgtacagcaagtatgacgccgccaccggtgaacgcttgaccaatgagttctttaccacgggcgataacaattggtactatattggttctaatggtaagaccgtaaccggtgaagtcaaaatcggtgcggacacctattactttgccaaagatggcaaacaggtcaagggccaaaccgtcaccgcaggcaatggccgcatctcctattactacggcgattctggtaagaaagcaatcagcacgtggatcgaaattcaaccgggtatctatgtctattttgataagacgggcatcgcgtacccaccgcgtgtgctgaattaa

SEQ ID NO: 30 - protein, 1341 aa, Streptococcus salivarius
MTDGKYYYVNEDGSHKENFAITVNGQLLYFGKDGALTSSSTHSFTPGTTNIVDGFSINNRAYDSSEASFELINGYLTADSWYRPVSIIKDGVTWQASTAEDFRPLLMAWWPNVDTQVNYLNYMSKVFNLEAKYTSTDKQADLNRAAKDIQVKIEQKIQAEKSTQWLRETISAFVKTQPQWNKETENYSKGGGEDHLQGGALLYVNDSRTPWANSNYRLLNRTATNQTGTINKSVLDEQSDPNHMGGFDFLLANDVDLSNPVVQAEQLNQIHYLMNWGSIVMGDKDANFDGIRVDAVDNVNADMLQLYTNYFREYYGVNKSEAQALAHISVLEAWSLNDNHYNDKTDGAALAMENKQRLALLFSLAKPIKDRTPAVSPLYNNTFNTTQRDFKTDWINKDGSTAYNEDGTAKQSTIGKYNEKYGDASGNYVFIRAHDNNVQDIIAEIIKKEINKKSDGFTISDSEMKQAFEIYNKDMLSSNKKYTLNNIPAAYAVMLQNMETITRVYYGDLYTDDGHYMETKSPYHDTIVNLMKNRIKYVSGGQAQRSYWLPTDGKMDNSDVELYRTSEVYTSVRYGKDIMTADDTEGSKYSRTSGQVTLVVNNPKLTLHESAKLNVEMGKIHANQKYRALIVGTADGIKNFTSDAEAIAAGYVKETDSNGVLTFGANDIKGYETFDMSGFVAVWVPVGASDDQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANSKSSGRDYQAQYGGEFLAELKAKYPKMFTENMISTGKPIDDSVKLKQWKAKYFNGTNVLDRGVGYVLSDEATGKYFTVTKEGNFIPLQLTGNEKAVTGFSNDGKGITYFGTSGNQAKSAFVTFNGNTYYFDARGHMVTNGEYSPNGKDVYRFLPNGIMLSNAFYVDANGNTYLYNYKGQMYKGGYTKFDVTETDKDGNESKVVKFRYFTNEGVMAKGLTVIDGSTQYFGEDGFQTKDKLATYKGKTYYFEAHTGNAIKNTWRNIDGKWYHFDENGVAATGAQVINGQKLYFNEDGSQVKGGVVKNADGTYSKYKEGSGELVTNEFFTTDGNVWYYAGADGKTVTGAQVINGQHLYFKEDGSQVKGGVVKNADGTYSKYDAATGERLTNEFFTTGDNNWYYIGSNGKTVTGEVKIGADTYYFAKDGKQVKGQTVTAGNGRISYYYGDSGKKAISTWIEIQPGIYVYFDKTGIAYPPRVLN

SEQ ID NO: 31-32 - skipped (2 entries)

SEQ ID NO: 33 - DNA, 4026 bp, Streptococcus salivarius
  source 1..4026: mol_type = unassigned DNA
atgatcgacggcaaatactactatgtaaacgaggacggcagccacaaagagaatttcgcgattacggtaaacggtcagctgctgtactttggtaaggacggtgctctgacgagcagctccacgtacagctttaccccgggtacgaccaatattgtcgatggcttcagcattaacaaccgtgcgtatgacagcagcgaggcatcctttgagctgatcgatggttatttgaccgcggatagctggtatcgtccggcgagcatcattaaggacggcgttacgtggcaggcctcgaccgcagaagattttcgtccgctgctgatggcttggtggccgaatgttgacacccaggtgaattatctgaattacatgtccaaggttttcaacctggatgcaaagtacaccagcaccgacaagcaggaaaccctgaacgtggctgcgaaagatatccaagtcaagattgagcaaaagattcaggcagagaaatctacccagtggctgcgtgaaacgattagcgcgtttgttaaaactcagccgcaatggaataaagaaacggaaaactattccaagggtggtggcgaggaccatctgcaaggcggtgccctgttgtacgttaacgattcgcgcaccccgtgggcgaactcgaactatcgcttgctgaaccataccgctaccaatcaaaaaggcactattgacaaatctgtcctggacgagcagagcgacccgaaccacatgggcggtttcgattttctgctggcgaacgacgtcgacctgagcaacccggtggtgcaggccgaacaactgaaccagattcactacctgatgaattggggtagcatcgtgatgggtgataaagatgcgaactttgacggcattcgtgtcgatgcggtcgataacgtggacgccgacatgttgcagctgtacacgaactactttcgtgagtactacggcgttaacaagagcgaagcaaatgccctggcgcatatcagcgttctggaagcgtggagcctgaatgacaatcactataacgataagacggacggtgcggccctggcaatggagaataaacaacgtctggcgctgctgttcagcctggcgaaaccgatcaaagagcgtacgccggctgtgagcccactgtataacaacaccttcaatactacgcagcgtgacgagaaaacggactggattaacaaagacggtagcaaagcgtataacgaggatggtaccgtcaagcaatcgaccattggtaagtacaatgagaagtatggcgacgcaagcggtaattacgtgttcattcgtgcccacgacaacaatgttcaagacatcatcgccgaaatcatcaagaaagagatcaaccctaagagcgacggtttcaccatcaccgacgcagagatgaagaaggcctttgaaatctacaacaaggacatgttgagcagcgataagaagtatactctgaacaacattccggctgcgtacgcggtgatgttgcagaatatggaaaccatcacgcgtgtttactatggtgatctgtataccgataatggcaactacatggaaacgaaaagcccgtactatgacaccattgttaatctgatgaagaatcgcatcaagtatgtgtctggcggtcaagcgcagcgttcttactggctgccgaccgatggtaagatggacaatagcgatgtggaactgtaccgcaccaacgaggtatacgcttctgtgcgctatggtaaagacattatgaccgccgatgataccgagggttccaagtactcccgtacgagcggccaagttaccttggtggcaaacaacccgaaattgaccctggaccaaagcgcgaaactgaaagtggagatgggtaagatccacgcaaatcaaaagtaccgtgcactgattgtcggtaccgccgacggtatcaagaatttcaccagcgatgcggatgcgattgcagcaggctatgttaaagagactgatagcaatggtgtgctgacgtttggtgcgaacgacattaaaggctatgaaacgtttgacatgagcggtttcgttgcggtgtgggtgcctgtgggtgctagcgatgatcaggatatccgtgtcgcgccgagcaccgaggcaaagaaagaaggtgagctgacgttgaaagcgaccgaggcctatgacagccagttgatttacgaaggtttcagcaatttccaaaccattccagacggttccgatccgagcgtctacaccaatcgcaaaatcgcggaaaacgttgatctgttcaaaagctggggtgtgaccagcttcgaaatggcaccgcaattcgttagcgcggacgatggtacgttcttggacagcgttatccaaaatggctatgcgttcgccgatcgttatgacttggcgatgagcaaaaacaacaaatacggcagcaaagaggatctgcgcgacgccctgaaagcgctgcataaagcgggtattcaagccatcgctgactgggttccggaccagatctaccagctgccgggtaaagaagtcgttaccgcgacccgcaccgatggcgctggccgtaagatcgcggatgcaattatcgatcatagcttgtatgtggccaatactaaaagctccggtaaggattaccaggcgaaatatggtggtgaatttctggctgagctgaaggccaaatacccggagatgttcaaggtcaacatgattagcaccggcaaacctattgatgactctgtcaaattgaaacaatggaaggcagagtatttcaatggcactaacgtcctggaacgtggtgttggttacgtgctgagcgacgaggcgaccggtaaatacttcaccgttacgaaggacggcaatttcatcccgctgcaactgaccggtaatgagaaggttgtgacgggtttttctaatgacggtaagggcattacctacttcggtacctcgggtacccaggcaaagagcgcattcgtgacgtttaacggtaacacctactactttgatgcacgcggccacatggtgacgaacggcgagtacagcccgaacggcaaggatgtttatcgcttcctgccgaatggcatcatgctgtccaatgcgttttacgtcgatgcaaatggtaatacttacctgtacaacagcaagggtcagatgtataagggcggttataccaagttcgacgttactgaaacggacaaggacggtaaagagagcaaagtagtgaagtttcgttatttcacgaacgaaggcgtcatggcgaaaggtgtcaccgttattgatggctttacccagtatttcggtgaagatggctttcaagcgaaggacaagctggtgacctttaagggcaaaacctactattttgacgcgcacacgggcaacgccatcaagaacacctggcgtaatatcgacggtaagtggtatcattttgatgcgaacggtgtggcggcgaccggcgcacaggtcattaatggtcaaaaactgtactttaatgaggacggtagccaagtcaaaggtggcgtcgtcaagaatgcagatggcacctatagcaaatacaaagagggctccggtgagctggttaccaacgagttctttaccacggatggtaacgtctggtactatgctggtgcgaatggcaagaccgttaccggtgcacaggttatcaacggccagcacctgtacttcaatgcggatggctctcaagtgaagggcggtgtcgtcaaaaacgcggacggtacgtactccaaatacgatgccgcgaccggtgaacgtctgaccaatgagtttttcacgactggtgacaacaattggtactacatcggcgccaacggtaagacggttacgggcgaagtgaaaattggcgacgatacgtactacttcgcaaaagatggtaaacaggtgaaaggtcagacggtttccgctggtaatggccgcatcagctactattacggtgactctggtaaacgtgcggttagcacgtgggttgaaattcaaccgggcgtgtatgtctattttgataagaatggcctggcatatccaccgcgcgttttgaattaa

SEQ ID NO: 34 - protein, 1341 aa, Streptococcus salivarius
MIDGKYYYVNEDGSHKENFAITVNGQLLYFGKDGALTSSSTYSFTPGTTNIVDGFSINNRAYDSSEASFELIDGYLTADSWYRPASIIKDGVTWQASTAEDFRPLLMAWWPNVDTQVNYLNYMSKVFNLDAKYTSTDKQETLNVAAKDIQVKIEQKIQAEKSTQWLRETISAFVKTQPQWNKETENYSKGGGEDHLQGGALLYVNDSRTPWANSNYRLLNHTATNQKGTIDKSVLDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSLNDNHYNDKTDGAALAMENKQRLALLFSLAKPIKERTPAVSPLYNNTFNTTQRDEKTDWINKDGSKAYNEDGTVKQSTIGKYNEKYGDASGNYVFIRAHDNNVQDIIAEIIKKEINPKSDGFTITDAEMKKAFEIYNKDMLSSDKKYTLNNIPAAYAVMLQNMETITRVYYGDLYTDNGNYMETKSPYYDTIVNLMKNRIKYVSGGQAQRSYWLPTDGKMDNSDVELYRTNEVYASVRYGKDIMTADDTEGSKYSRTSGQVTLVANNPKLTLDQSAKLKVEMGKIHANQKYRALIVGTADGIKNFTSDADAIAAGYVKETDSNGVLTFGANDIKGYETFDMSGFVAVWVPVGASDDQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANTKSSGKDYQAKYGGEFLAELKAKYPEMFKVNMISTGKPIDDSVKLKQWKAEYFNGTNVLERGVGYVLSDEATGKYFTVTKDGNFIPLQLTGNEKVVTGFSNDGKGITYFGTSGTQAKSAFVTFNGNTYYFDARGHMVTNGEYSPNGKDVYRFLPNGIMLSNAFYVDANGNTYLYNSKGQMYKGGYTKFDVTETDKDGKESKVVKFRYFTNEGVMAKGVTVIDGFTQYFGEDGFQAKDKLVTFKGKTYYFDAHTGNAIKNTWRNIDGKWYHFDANGVAATGAQVINGQKLYFNEDGSQVKGGVVKNADGTYSKYKEGSGELVTNEFFTTDGNVWYYAGANGKTVTGAQVINGQHLYFNADGSQVKGGVVKNADGTYSKYDAATGERLTNEFFTTGDNNWYYIGANGKTVTGEVKIGDDTYYFAKDGKQVKGQTVSAGNGRISYYYGDSGKRAVSTWVEIQPGVYVYFDKNGLAYPPRVLN

SEQ ID NO: 35-58 - skipped (24 entries)

SEQ ID NO: 59 - protein, 1242 aa, Streptococcus sp.
MINGKEYYVEDDGTVRKNYVLERNGGSQYFNAETGELSNQKDYRFDKNGGTGSAADSTTNTNVTVNGDKNAFYGTTEKDIELVDGYFTANTWYRPKEILKDGKEWTASTENDKRPLLTVWWPSKAIQASYLNYMREEGLGTNQTFTSYSSQTQMDQAALEVQKRIEERIAREGNTDWLRTTIKNFVKTQPGWNSTSENLDNSDHLQGGALLYNNSNRTSYANSDYRLLNRTPTQQDGTRRYFKDNSSGGFEFLLANDIDNSNPAVQAEQLNWLHYIMNIGSLTGGSEDENFDGVRVDAVDNVNADLLQIASDYFKAKYGVEKSEEEAIKHLSILEAWSHNDAYYNEDTKGAQLPMDDPLRLAMVFSFLRPIGNRSGLEPLITNSLNDRSESKKNTKRMANYTFVRAHDSEVQSVIGQIIKNEINPQSTGNTFTLDEMKKAFKIYNADMRSANKRYTQYNIPSAYAFMLTNKDTVPRVYYGDLYTDDGQYMAQKSPYHDAISTLLQARIRYAAGGQDMKMSYVGSGNTNGWDASGVLTSVRYGKGANNASDAGTAETRNQGMAVILSNQPALRLNSNLTINMGAAHRNQAYRPLLLTTSNGVASYLNDGDANGIVKYTDANGYLTFNPGEISGVRNAQVDGYLAVWVPLGASENQDVRVAASKSKNSSGLVYDSSAALDSQVIYEGFSNFQDFVQDPSQYTNKKIAENANLFKSWGITSFEFAPQYVSSDDGTFLDSVIQNGYAFSDRYDIGMSKDNKYGSLADLKAALKSLHAVGISAIADWVPDQIYNLPGDEVVTATRVNNYGETKDGAIIDHSLYVAKTRTFGNDYQGKYGGAYLDELKRLYPQFFDRVQISTGKRLTTDEKITKWSAKYMNGTNILDRGSEYVLKNGLSGYYGTNGGKVSLPKVVGSNQSTNNNNQNGDGSGRFEKSWGSVYYRYNDGQRARNAFIKDNDGNVYYFDNTGRMAIGEKTIDGKQYFFLANGVQLRDGYRQNRRGQVFYYDENGIMSQTGKPSPKPEPKPDNNTFSRNQFIQIGNNVWAYYDGNGKRVIGRQNINGQELFFDNNGVQVKGRTAQVDGVTRYFDANSGEMARNRFAEVEPGVWAYFNNDGAAVTGSQNINGQTLYFDQNGHQVKGALVTVDGNLRYYDANSGDLYRNRFQEVNGSWYYFDGNGNAVKGMVNINGQNLLFDNDGKQVKGHLVRVNGVIRYYDPNSGEMAVNRWVEISSGWWVYFDGEGRGQI

SEQ ID NO: 60 - protein, 1518 aa, Streptococcus salivarius
MENKIHYKLHKVKKQWVTIAVASVALATVLGGLSVTTSSVSADETQDKTVTQSNSGTTASLVTSPEATKEADKRTNTKEADVLTPAKETNAVETATTTNTQATAEAATTATTADVAVAAVPNKEAVVTTDAPAVTTEKAEEQPATVKAEVVNTEVKAPEAALKDSEVEAALSLKNIKNIDGKYYYVNEDGSHKENFAITVNGQLLYFGKDGALTSSSTYSFTPGTTNIVDGFSINNRAYDSSEASFELIDGYLTADSWYRPASIIKDGVTWQASTAEDFRPLLMAWWPNVDTQVNYLNYMSKVFNLDAKYSSTDKQETLKVAAKDIQIKIEQKIQAEKSTQWLRETISAFVKTQPQWNKETENYSKGGGEDHLQGGALLYVNDSRTPWANSDYRRLNRTATNQTGTIDKSILDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSLNDNHYNDKTDGAALAMENKQRLALLFSLAKPIKERTPAVSPLYNNTFNTTQRDEKTDWINKDGSKAYNEDGTVKQSTIGKYNEKYGDASGNYVFIRAHDNNVQDIIAEIIKKEINPKSDGFTITDAEMKQAFEIYNKDMLSSDKKYTLNNIPAAYAVMLQNMETITRVYYGDLYTDDGHYMETKSPYYDTIVNLMKSRIKYVSGGQAQRSYWLPTDGKMDNSDVELYRTNEVYTSVRYGKDIMTANDTEGSKYSRTSGQVTLVANNPKLNLDQSAKLNVEMGKIHANQKYRALIVGTADGIKNFTSDADAIAAGYVKETDSNGVLTFGANDIKGYETFDMSGFVAVWVPVGASDNQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANSKSSGKDYQAKYGGEFLAELKAKYPEMFKVNMISTGKPIDDSVKLKQWKAEYFNGTNVLERGVGYVLSDEATGKYFTVTKEGNFIPLQLTGKEKVITGFSSDGKGITYFGTSGTQAKSAFVTFNGNTYYFDARGHMVTNSEYSPNGKDVYRFLPNGIMLSNAFYIDANGNTYLYNSKGQMYKGGYTKFDVSETDKDGKESKVVKFRYFTNEGVMAKGVTVIDGFTQYFGEDGFQAKDKLVTFKGKTYYFDAHTGNGIKDTWRNINGKWYYFDANGVAATGAQVINGQKLYFNEDGSQVKGGVVKNADGTYSKYKEGFGELVTNEFFTTDGNVWYYAGANGKTVTGAQVINGQHLYFNADGSQVKGGVVKNADGTYSKYNASTGERLTNEFFTTGDNNWYYIGANGKSVTGEVKIGDDTYFFAKDGKQVKGQTVSAGNGRISYYYGDSGKRAVSTWIEIQPGVYVYFDKNGLAYPPRVLN

SEQ ID NO: 61 - protein, 1528 aa, Streptococcus salivarius
MTNKITGKIIMENKVHYKLHKVKKQWVTIAVASAALATVVGGLSATTSSVSADETQDKIVTQPNLDTTADLVTSTEATKEVDKRTNTKEADVLTPAKETNAVETATTTNTQATAEAATTATTSDVAVAAVPNKEAVVTTDAPAVTTEKAEEQPATVKAEVVNTEVKAPQAALKDSEVEAALSLKNIKYTDGKYYYVNEDGSHKENFAITVNGQLLYFGKDGALTSSSTHSFTPGTTNIVDGFSINNRAYDSSEASFELINGYLTADSWYRPVSIIKDGVTWQASTAEDFRPLLMAWWPNVDTQVNYLNYMSKVFNLEAKYTSTDKQADLNRAAKDIQVKIEQKIQAEKSTQWLRETISAFVKTQPQWNKETENYSKGGGEDHLQGGALLYVNDSRTPWANSNYRLLNRTATNQTGTINKSVLDEQSDPNHMGGFDFLLANDVDLSNPVVQAEQLNQIHYLMNWGSIVMGDKDANFDGIRVDAVDNVNADMLQLYTNYFREYYGVNKSEAQALAHISVLEAWSLNDNHYNDKTDGAALAMENKQRLALLFSLAKPIKDRTPAVSPLYNNTFNTTQRDFKTDWINKDGSTAYNEDGTAKQSTIGKYNEKYGDASGNYVFIRAHDNNVQDIIAEIIKKEINKKSDGFTISDSEMKQAFEIYNKDMLSSNKKYTLNNIPAAYAVMLQNMETITRVYYGDLYTDDGHYMETKSPYHDTIVNLMKNRIKYVSGGQAQRSYWLPTDGKMDNSDVELYRTSEVYTSVRYGKDIMTADDTEGSKYSRTSGQVTLVVNNPKLTLHESAKLNVEMGKIHANQKYRALIVGTADGIKNFTSDAEAIAAGYVKETDSNGVLTFGANDIKGYETFDMSGFVAVWVPVGASDDQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANSKSSGRDYQAQYGGEFLAELKAKYPKMFTENMISTGKPIDDSVKLKQWKAKYFNGTNVLDRGVGYVLSDEATGKYFTVTKEGNFIPLQLTGNEKAVTGFSNDGKGITYFGTSGNQAKSAFVTFNGNTYYFDARGHMVTNGEYSPNGKDVYRFLPNGIMLSNAFYVDANGNTYLYNYKGQMYKGGYTKFDVTETDKDGNESKVVKFRYFTNEGVMAKGLTVIDGSTQYFGEDGFQTKDKLATYKGKTYYFEAHTGNAIKNTWRNIDGKWYHFDENGVAATGAQVINGQKLYFNEDGSQVKGGVVKNADGTYSKYKEGSGELVTNEFFTTDGNVWYYAGADGKTVTGAQVINGQHLYFKEDGSQVKGGVVKNADGTYSKYDAATGERLTNEFFTTGDNNWYYIGSNGKTVTGEVKIGADTYYFAKDGKQVKGQTVTAGNGRISYYYGDSGKKAISTWIEIQPGIYVYFDKTGIAYPPRVLN

SEQ ID NO: 62 - protein, 1518 aa, Streptococcus salivarius
MENKIHYKLHKVKKQWVTIAVASVALATVLGGLSVTTSSVSADETQDKTVTQSNSGTTASLVTSPEATKEADKRTNTKEADVLTPAKETNAVETATTTNTQATAEAATTATTADVAVAAVPNKEAVVTTDAPAVTTEKAEEQPATVKAEVVNTEVKAPEAALKDSEVEAALSLKNIKNIDGKYYYVNEDGSHKENFAITVNGQLLYFGKDGALTSSSTYSFTPGTTNIVDGFSINNRAYDSSEASFELIDGYLTADSWYRPASIIKDGVTWQASTAEDFRPLLMAWWPNVDTQVNYLNYMSKVFNLDAKYSSTDKQETLKVAAKDIQIKIEQKIQAEKSTQWLRETISAFVKTQPQWNKETENYSKGGGEDHLQGGALLYVNDSRTPWANSDYRRLNRTATNQTGTIDKSILDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSLNDNHYNDKTDGAALAMENKQRLALLFSLAKPIKERTPAVSPLYNNTFNTTQRDEKTDWINKDGSKAYNEDGTVKQSTIGKYNEKYGDASGNYVFIRAHDNNVQDIIAEIIKKEINPKSDGFTITDAEMKQAFEIYNKDMLSSDKKYTLNNIPAAYAVMLQNMETITRVYYGDLYTDDGHYMETKSPYYDTIVNLMKSRIKYVSGGQAQRSYWLPTDGKMDNSDVELYRTNEVYTSVRYGKDIMTANDTEGSKYSRTSGQVTLVANNPKLTLDQSAKLNVEMGKIHANQKYRALIVGTADGIKNFTSDADAIAAGYVKETDSNGVLTFGANDIKGYETFDMSGFVAVWVPVGASDDQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANTKSSGKDYQAKYGGEFLAELKAKYPEMFKVNMISTGKPIDDSVKLKQWKAEYFNGTNVLERGVGYVLSDEATGKYFTVTKDGNFIPLQLTGNEKVVTGFSNDGKGITYFGTSGTQAKSAFVTFNGNTYYFDARGHMVTNGEYSPNGKDVYRFLPNGIMLSNAFYVDANGNTYLYNSKGQMYKGGYTKFDVTETDKDGKESKVVKFRYFTNEGVMAKGVTVIDGFTQYFGEDGFQAKDKLVTFKGKTYYFDAHTGNAIKDTWRNINGKWYHFDANGVAATGAQVINGQKLYFNEDGSQVKGGVVKNADGTYSKYKEGSGELVTNEFFTTDGNVWYYAGANGKTVTGAQVINGQHLYFNADGSQVKGGVVKNADGTYSKYDASTGERLTNEFFTTGDNNWYYIGANGKSVTGEVKIGDDTYFFAKDGKQVKGQTVSAGNGRISYYYGDSGKRAVSTWIEIQPGVYVYFDKNGIAYPPRVLN

SEQ ID NO: 63 - protein, 1431 aa, Streptococcus salivarius
MTKETNTVDAATTTNTQAAADAATKTADAAVTALPNKEAVVTTDAPAVTTEKAAEQPATVKSEVVNTEVKAPEAALKDSEVEAALSLKNIKNIDGKYYYVNKDGSHKENFAITVNGQLLYFGKDGALTSSSTYSFTQGTTNIVDGFSKNNRAYDSSEASFELIDGYLTADSWYRPVSIIKDGVTWQASTKEDFRPLLMAWWPNVDTQVNYLNYMSKVFNLDAKYTSTDKQVDLNRAAKDIQVKIEQKIQAEKSTQWLREAISAFVKTQPQWNKETENFSKGGGEDHLQGGALLYVNDPRTPWANSNYRLLNRTATNQTGTIDKSVLDEQSDPNHMGGFDFLLANDVDTSNPVVQAEQLNQIHYLMNWGSIVMGDKDANFDGIRVDAVDNVDADMLQLYTNYFREYYGVNKSEANALAHISVLEAWSLNDNHYNDKTDGAALAMENKQRLALLFSLAKPIKERTPAVSPLYNNTFNTTQRDEKTDWINKDGSKAYNEDGTVKQSTIGKYNEKYGDASGNYVFIRAHDNNVQDIIAEIIKKEINPKSDGFTITDAEMKKAFEIYNKDMLSSDKKYTLNNIPAAYAVMLQNMETITRVYYGDLYTDDGHYMETKSPYYDTIVNLMKNRIKYVSGGQAQRSYWLPTDGKMDKSDVELYRTNEVYTSVRYGKDIMTADDTQGSKYSRTSGQVTLVVNNPKLSLDKSAKLDVEMGKIHANQKYRALIVGTPNGIKNFTSDAEAIAAGYVKETDGNGVLTFGANDIKGYETFDMSGFVAVWVPVGASDDQDIRVAASTAAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRNALKALHKAGIQAIADWVPDQIYQLPGKEVVTATRTDGAGRKISDAIIDHSLYVANSKSSGKDYQAKYGGEFLAELKAKYPEMFKVNMISTGKPIDDSVKLKQWKAEYFNGTNVLDRGVGYVLSDEATGKYFTVTKEGNFIPLQLKGNEKVITGFSSDGKGITYFGTSGNQAKSAFVTFNGNTYYFDARGHMVTNGEYSPNGKDVYRFLPNGIMLSNAFYVDGNGNTYLYNSKGQMYKGGYSKFDVTETKDGKESKVVKFRYFTNEGVMAKGVTVVDGFTQYFNEDGIQSKDELVTYNGKTYYFEAHTGNAIKNTWRNIKGKWYHFDANGVAATGAQVINGQHLYFNEDGSQVKGGVVKNADGTFSKYKDGSGDLVVNEFFTTGDNVWYYAGANGKTVTGAQVINGQHLFFKEDGSQVKGDFVKNSDGTYSKYDAASGERLTNEFFTTGDNHWYYIGANGKTVTGEVKIGDDTYFFAKDGKQLKGQIVTTRSGRISYYFGDSGKKAISTWVEIQPGVFVFFDKNGLAYPPENMN

SEQ ID NO: 64 - protein, 1532 aa, unidentified
  REGION 1..1532: note = unknown Streptococcus sp. C150
MENKVHYKLHKVKKQWVTIAVASAALATVVGGLSATTSSVSADETQDKTVTQPNSDTTADLVTSTEATKEVDKRTNTKEADVLTPAKETNTVETAATTNTQATAEAAKTATTTNTQATAEVAKTATTADVAVAAVPNKEAVVTTDAPAVTTEKAEEQPATVKAEVVNTEVKAPEAALKDSEVEAALSLKNIKNIDGKYYYVNEDGSHKENFAITVNGQLLYFGKDGALTSSSTYSFTQGTTNIVDGFSINNRAYDSSEASFELIDGYLTADSWYRPASIIKDGVTWQASTAEDFRPLLMAWWPNVDTQVNYLNYMSKVFNLDAKYSSTDKQETLKVAAKDIQIKIEQKIQAEKSTQWLRETISAFVKTQPQWNKETENYSKGGGEDHLQGGALLYVNDSRTPWANSNYRLLNRTATNQTGTIDKSILDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSLNDNHYNDKTDVAALAMENKQRLALLFSLAKPIKERTPAVSPLYNNTFNTTQRDEKTDWINKDGSKAYNEDGTVKKSTIGKYNEKYGDASGNYVFIRAHDNNVQDIIAEIIKKEINEKSDGFTITDSEMKRAFEIYNKDMLSNDKKYTLNNIPAAYAVMLQNMETITRVYYGDLYTDDGNYMEAKSPYYDTIVNLMKSRIKYVSGGQAQRSYWLPTDGKMDKSDVELYRTNEVYTSVRYGKDIMTADDTQGSKYSRTSGQVTLVVNNPKLTLDQSAKLNVVMGKIHANQKYRALIVGTPNGIKNFTSDAEAIAAGYVKETDGNGVLTFGANDIKGYETFDMSGFVAVWVPVGASDDQDIRVAASTAAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRNALKALHKAGIQAIADWVPDQIYQLPGKEVVTATRTDGAGRKISDAIIDHSLYVANSKSSGKDYQAKYGGEFLAELKAKYPEMFKVNMISTGKPIDDSVKLKQWKAEYFNGTNVLDRGVGYVLSDEATGKYFTVTKEGNFIPLQLKGNKKVITGFSSDGKGITYFGTSGNQAKSAFVTFNGNTYYFDARGHMVTNGEYSPNGKDVYRFLPNGIMLSNAFYVDGNGNTYLYNSKGQMYKGGYSKFDVTETKDGKESKVVKFRYFTNEGVMAKGVTVVDGFTQYFNEDGIQSKDELVTYNGKTYYFEAHTGNAIKNTWRNIKGKWYHFDANGVAATGAQVINGQHLYFNEDGSQVKGSIVKNADGTFSKYKDSSGDLVVNEFFTTGDNVWYYAGANGKTVTGAQVINGQHLFFKEDGSQVKGDFVKNSDGTYSKYDAASGERLTNEFFTTGDNHWYYIGANGKTVTGEVKIGDDTYFFAKDGKQLKGQIVTTRSGRISYYFGDSGKKAISTWVEIQPGVFVFFDKNGLAYPPENMN

SEQ ID NO: 65 - protein, 1341 aa, Streptococcus salivarius
MIDGKYYYVNEDGSHKENFAITVNGQLLYFGKDGALTSSSTYSFTPGTTNIVDGFSINNRAYDSSEASFELIDGYLTADSWYRPASIIKDGVTWQASTAEDFRPLLMAWWPNVDTQVNYLNYMSKVFNLDAKYSSTDKQETLKVAAKDIQIKIEQKIQAEKSTQWLRETISAFVKTQPQWNKETENYSKGGGEDHLQGGALLYVNDSRTPWANSDYRRLNRTATNQTGTIDKSILDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSLNDNHYNDKTDGAALAMENKQRLALLFSLAKPIKERTPAVSPLYNNTFNTTQRDEKTDWINKDGSKAYNEDGTVKQSTIGKYNEKYGDASGNYVFIRAHDNNVQDIIAEIIKKEINPKSDGFTITDAEMKQAFEIYNKDMLSSDKKYTLNNIPAAYAVMLQNMETITRVYYGDLYTDDGHYMETKSPYYDTIVNLMKSRIKYVSGGQAQRSYWLPTDGKMDNSDVELYRTNEVYTSVRYGKDIMTANDTEGSKYSRTSGQVTLVANNPKLNLDQSAKLNVEMGKIHANQKYRALIVGTADGIKNFTSDADAIAAGYVKETDSNGVLTFGANDIKGYETFDMSGFVAVWVPVGASDNQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANSKSSGKDYQAKYGGEFLAELKAKYPEMFKVNMISTGKPIDDSVKLKQWKAEYFNGTNVLERGVGYVLSDEATGKYFTVTKEGNFIPLQLTGKEKVITGFSSDGKGITYFGTSGTQAKSAFVTFNGNTYYFDARGHMVTNSEYSPNGKDVYRFLPNGIMLSNAFYIDANGNTYLYNSKGQMYKGGYTKFDVSETDKDGKESKVVKFRYFTNEGVMAKGVTVIDGFTQYFGEDGFQAKDKLVTFKGKTYYFDAHTGNGIKDTWRNINGKWYYFDANGVAATGAQVINGQKLYFNEDGSQVKGGVVKNADGTYSKYKEGFGELVTNEFFTTDGNVWYYAGANGKTVTGAQVINGQHLYFNADGSQVKGGVVKNADGTYSKYNASTGERLTNEFFTTGDNNWYYIGANGKSVTGEVKIGDDTYFFAKDGKQVKGQTVSAGNGRISYYYGDSGKRAVSTWIEIQPGVYVYFDKNGLAYPPRVLN

(19) United States
(12) Patent Application Publication
(10) Pub. No.: US 2024/0309120 A1
(43) Pub. Date: Sep. 19, 2024

(54) ALPHA-1,3-GLUCAN GRAFT COPOLYMERS

(71) Applicant: NUTRITION & BIOSCIENCES USA 4, INC., Rochester, NY (US)

(72) Inventors: RONG GUAN, WILMINGTON, DE (US); MICHAEL D. GAGNON, WEST GROVE, PA (US); NIKITA ILTCHENKO, WILMINGTON, DE (US); YEFIM BRUN, WILMINGTON, DE (US); LAURIE A. HOWE, BEAR, DE (US)

(21) Appl. No.: 18/539,541

(22) Filed: Dec. 14, 2023

Related U.S. Application Data
(63) Continuation of application No. 16/663,830, filed on Oct. 25, 2019, now Pat. No. 11,859,022.
(60) Provisional application No. 62/750,520, filed on Oct. 25, 2018.

(51) Int. Cl. C08B 37/00; C12N 9/10; C12P 19/04; C12P 19/18

(52) U.S. Cl. CPC C08B 37/0009; C12N 9/1048; C12P 19/04; C12P 19/18

(57) ABSTRACT

Compositions are disclosed herein comprising a graft copolymer that comprises: (i) a backbone comprising an alpha-1,3-glucan ether or ester compound, and (ii) one or more alpha-1,3-glucan side chains comprising at least about 50% alpha-1,3 glycosidic linkages. Further disclosed are reactions for producing such graft copolymers, as well as their use in various applications.

This application is a continuation of application Ser. No. 16/663,830 (filed Oct. 25, 2019) (now U.S. Pat. No. 11,859,022), which claims the benefit of U.S. Provisional Application No. 62/750,520 (filed Oct. 25, 2018), both of which prior applications are incorporated herein by reference in their entirety.

### FIELD

The present disclosure is in the field of polysaccharides. For example, the disclosure pertains to compositions comprising alpha-1,3-glucan derivative/alpha-1,3-glucan graft copolymers and use thereof in various applications.

### REFERENCE TO SEQUENCE LISTING SUBMITTED ELECTRONICALLY

The official copy of the sequence listing is submitted electronically via EFS-Web or Patent Center as a file named CL6595USCNT_SequenceListing.xml created on Jun. 6, 2024 and having a size of about 122 kilobytes. The sequence listing contained in this file is part of the specification and herein incorporated by reference in its entirety.

### BACKGROUND

Driven by a desire to use polysaccharides in various applications, researchers have explored for polysaccharides that are biodegradable and that can be made economically from renewably sourced feedstocks. One such polysaccharide is alpha-1,3-glucan, an insoluble glucan polymer characterized by having alpha-1,3-glycosidic linkages. This polymer has been prepared, for example, using a glucosyltransferase enzyme isolated from Streptococcus salivarius (Simpson et al., Microbiology 141:1451-1460, 1995). Also for example, U.S. Pat. No. 7,000,000 disclosed the preparation of a spun fiber from enzymatically produced alpha-1,3-glucan. Various other glucan materials have also been studied for developing new or enhanced applications. For example, U.S. Patent Appl. Publ. No. 2015/0232819 discloses enzymatic synthesis of several insoluble glucans having mixed alpha-1,3 and-1,6 linkages.

Despite this work, new forms of alpha-1,3-glucan are desired to enhance the economic value and performance characteristics of this material in various applications. Compositions comprising alpha-1,3-glucan in the form of a graft copolymer are presently disclosed to address this need.

### SUMMARY

In one embodiment, the present disclosure concerns a composition comprising a graft copolymer that comprises: (i) a backbone comprising an alpha-1,3-glucan ether or ester compound that has a degree of substitution (DoS) of about 0.001 to about 3.0, and (ii) one or more alpha-1,3-glucan side chains comprising at least about 50% alpha-1,3 glycosidic linkages.

In another embodiment, the present disclosure concerns a method of producing a graft copolymer, the method comprising: (a) contacting (in the context of a reaction composition) at least (i) water, (ii) sucrose, (iii) an alpha-1,3-glucan ether or ester compound that has a degree of substitution (DoS) of about 0.001 to about 3.0, and (iv) a glucosyltransferase enzyme that synthesizes alpha-1,3-glucan comprising at least about 50% alpha-1,3 glycosidic linkages, whereby a graft copolymer as presently disclosed is produced, optionally wherein the viscosity of the reaction composition increases by at least 10% at least 1 hour following the contacting step; and (b) optionally, isolating the graft copolymer produced in step (a).

In another embodiment, the present disclosure concerns a method of providing an aqueous composition, the method comprising: (a) providing a graft copolymer as presently disclosed and (b) dispersing or dissolving the graft copolymer into an aqueous liquid, thereby producing the aqueous composition.

### BRIEF DESCRIPTION OF THE DRAWINGS AND SEQUENCES

FIGS. 1A-B: Shear viscosities of 2-hour whole samples of glucosyltransferase reactions with 5 g/L CMG25 primer and 50 g/L sucrose (FIG. 1A) or 100 g/L sucrose (FIG. 1B). Logarithmic scales are used for the y-axes of these figures. Refer to Example 1.

FIG. 2: Shear viscosities of 0- and 24-hour whole samples of glucosyltransferase reaction with 10 g/L CMG25 primer and 22.5 g/L sucrose. A logarithmic scale is used for the y-axis. Refer to Example 2.

FIGS. 3A-D: Shear viscosities of various aqueous preparations: Samples 1 and 3 (FIG. 3A), Samples 2 and 4 (FIG. 3B), Samples 7 and 8 (FIG. 3C), and Samples 5-7 (FIG. 3D). Logarithmic scales are used for the y-axes of these figures. Refer to Example 3 and Table 5 therein.

FIG. 4: Shear viscosities of 24-hour whole samples of glucosyltransferase reactions with 10 g/L CMG31 primer and 10 or 20 g/L sucrose. A logarithmic scale is used for the y-axis. Refer to Example 4.

FIG. 5: Shear viscosities of 24-hour whole samples of glucosyltransferase reactions with 10 g/L CMG70 primer and 10 or 20 g/L sucrose. A logarithmic scale is used for the y-axis. Refer to Example 5.

TABLE 1
Summary of Nucleic Acid and Protein SEQ ID Numbers^{b}

| | Nucleic acid | Protein |
|---|---|---|
| Description | SEQ ID NO. | SEQ ID NO. |
| GTF 0874, Streptococcus sobrinus. The first 156 amino acids | 1 ^{a} | 2 |
| of the protein are deleted compared to GENBANK | | (1435 aa) |
| Identification No. 450874; a start methionine is included. | | |
| GTF 6855, Streptococcus salivarius SK126. The first 178 | 3 ^{a} | 4 |
| amino acids of the protein are deleted compared to | | (1341 aa) |
| GENBANK Identification No. 228476855 (Acc. No. | | |
| ZP_04061500.1); a start methionine is included. | | |
| GTF 2379, Streptococcus salivarius. The first 203 amino | 5 ^{a} | 6 |
| acids of the protein are deleted compared to GENBANK | | (1247 aa) |
| Identification No. 662379; a start methionine is included. | | |
| GTF 7527 or GTFJ, Streptococcus salivarius. The first 42 | 7 ^{a} | 8 |
| amino acids of the protein are deleted compared to | | (1477 aa) |
| GENBANK Identification No. 47527; a start methionine is | | |
| included. | | |
| GTF 1724, Streptococcus downei. The first 162 amino acids | 9 ^{a} | 10 |
| of the protein are deleted compared to GENBANK | | (1436 aa) |
| Identification No. 121724; a start methionine is included. | | |
| GTF 0544, Streptococcus mutans. The first 164 amino acids | 11 ^{a} | 12 |
| of the protein are deleted compared to GENBANK | | (1313 aa) |
| Identification No. 290580544; a start methionine is included. | | |
| GTF 5926, Streptococcus dentirousetti. The first 144 amino | 13 ^{a} | 14 |
| acids of the protein are deleted compared to GENBANK | | (1323 aa) |
| Identification No. 167735926; a start methionine is included. | | |
| GTF 4297, Streptococcus oralis. The first 228 amino acids of | 15 ^{a} | 16 |
| the protein are deleted compared to GENBANK Identification | | (1348 aa) |
| No. 7684297; a start methionine is included. | | |
| GTF 5618, Streptococcus sanguinis. The first 223 amino | 17 ^{a} | 18 |
| acids of the protein are deleted compared to GENBANK | | (1348 aa) |
| Identification No. 328945618; a start methionine is included. | | |
| GTF 2765, unknown Streptococcus sp. C150. The first 193 | 19 ^{a} | 20 |
| amino acids of the protein are deleted compared to | | (1340 aa) |
| GENBANK Identification No. 322372765; a start methionine is | | |
| included. | | |
| GTF 0427, Streptococcus sobrinus. The first 156 amino acids | 25 ^{a} | 26 |
| of the protein are deleted compared to GENBANK | | (1435 aa) |
| Identification No. 940427; a start methionine is included. | | |
| GTF 2919, Streptococcus salivarius PS4. The first 92 amino | 27 ^{a} | 28 |
| acids of the protein are deleted compared to GENBANK | | (1340 aa) |
| Identification No. 383282919; a start methionine is included. | | |
| GTF 2678, Streptococcus salivarius K12. The first 188 amino | 29 ^{a} | 30 |
| acids of the protein are deleted compared to GENBANK | | (1341 aa) |
| Identification No. 400182678; a start methionine is included. | | |
| GTF 3929, Streptococcus salivarius JIM8777. The first 178 | 33 ^{a} | 34 |
| amino acids of the protein are deleted compared to | | (1341 aa) |
| GENBANK Identification No. 387783929; a start methionine is | | |
| included. | | |
| GTF 3298, Streptococcus sp. C150. The first 209 amino | | 59 |
| acids of the protein are deleted compared to GENBANK | | (1242 aa) |
| Identification No. 322373298; a start methionine is included. | | |
| Wild type GTFJ, Streptococcus salivarius. GENBANK | | 60 |
| Identification No. 47527. | | (1518 aa) |
| Wild type GTF corresponding to GTF 2678, Streptococcus | | 61 |
| salivarius K12. | | (1528 aa) |
| Wild type GTF corresponding to GTF 6855, Streptococcus | | 62 |
| salivarius SK126. | | (1518 aa) |
| Wild type GTF corresponding to GTF 2919, Streptococcus | | 63 |
| salivarius PS4. | | (1431 aa) |
| Wild type GTF corresponding to GTF 2765, unknown | | 64 |
| Streptococcus sp. C150. | | (1532 aa) |
| Shorter version of GTF 7527, Streptococcus salivarius, (also | | 65 |
| referred to as “7527-NT” herein. The first 178 amino acids of | | (1341 aa) |
| the protein are deleted compared to GENBANK Identification | | |
| No. 47527; a start methionine is included. | | |

^{a} This DNA coding sequence is codon-optimized for expression in E. coli, and is merely disclosed as an example of a suitable coding sequence.
^{b}SEQ ID NOs: 21-24, 31, 32 and 35-58 are intentionally not included in this table and merely serve as placeholders.

### DETAILED DESCRIPTION

The disclosures of all cited patent and non-patent literature are incorporated herein by reference in their entirety.

Unless otherwise disclosed, the terms “a” and “an” as used herein are intended to encompass one or more (i.e., at least one) of a referenced feature.

Where present, all ranges are inclusive and combinable, except as otherwise noted. For example, when a range of “1 to 5” (i.e., 1-5) is recited, the recited range should be construed as including ranges “1 to 4”, “1 to 3”, “1-2”, “1-2 & 4-5”, “1-3 & 5”, and the like.

The terms “graft copolymer”, “branched copolymer” and the like herein generally refer to a copolymer comprising a “backbone” (or “main chain”) and side chains branching from the backbone. Examples of graft copolymers herein have a backbone comprising an alpha-1,3-glucan ether or ester compound, and at least one side chain of alpha-1,3-glucan comprising at least about 50% alpha-1,3 glycosidic linkages. In some aspects, a backbone can have an alpha-1,3-glucan extension, since the non-reducing end of the ether/ester glucan backbone is contemplated to be able to prime alpha-1,3-glucan synthesis by a glucosyltransferase enzyme. A backbone can be a [non-derivatized alpha-1,3-glucan]-[alpha-1,3-glucan ether/ester derivative] linear copolymer in some instances. An alpha-1,3-glucan ether or ester compound of a backbone herein can optionally be referred to in shorthand as an “alpha-glucan derivative”. In an alternative aspect of the present disclosure, any feature herein characterizing a graft copolymer can likewise characterize (if appropriate and applicable) a [non-derivatized alpha-1,3-glucan]-[alpha-1,3-glucan ether/ester derivative] linear copolymer that does not have any alpha-1,3-glucan side chains (such a copolymer is not a graft copolymer).

The terms “alpha-1,3-glucan side chain” and “alpha-1,3-glucan arm” and the like can be used interchangeably herein. An alpha-1,3-glucan side chain(s) is contemplated to be (i) joined directly to a glucose unit of the backbone via alpha-glycosidic linkage (e.g., alpha-1,6, alpha-1,4, or alpha-1,2) (in some cases, such linkage might result from the promiscuous activity of an alpha-1,3-glucan-synthesizing glucosyltransferase enzyme); (ii) extensions of pre-existing branches (e.g., alpha-1,2,-1,4, and/or-1,6), as such branches present non-reducing ends that can possibly prime alpha-1,3-glucan synthesis by a glucosyltransferase enzyme; and/or (iii) possibly joined in an uncharacterized manner.

The terms “alpha-glucan”, “alpha-glucan polymer” and the like are used interchangeably herein. An alpha-glucan is a polymer comprising glucose monomeric units linked together by alpha-glycosidic linkages. In typical embodiments, an alpha-glucan herein comprises at least about 80%, 81%, 82%, 83%, 84%, 85%, 86%, 87%, 88%, 89%, 90%, 91%, 92%, 93%, 94%, 95%, 96%, 97%, 98%, 99%, or 100% alpha-glycosidic linkages. Examples of alpha-glucan polymers herein include alpha-1,3-glucan used to prepare an ether or ester derivative, which is consequently used as a backbone in a graft copolymer, and alpha-1,3-glucan side arms of a graft copolymer.

The terms “poly alpha-1,3-glucan”, “alpha-1,3-glucan”, “alpha-1,3-glucan polymer” and the like are used interchangeably herein. Alpha-1,3-glucan is a polymer comprising glucose monomeric units linked together by glycosidic linkages, wherein at least about 30% of the glycosidic linkages are alpha-1,3. Alpha-1,3-glucan in certain embodiments comprises at least about 90% or 95% alpha-1,3 glycosidic linkages. Most or all of the other linkages in alpha-1,3-glucan herein typically are alpha-1,6, though some linkages may also be alpha-1,2 and/or alpha-1,4. Alpha-1,3-glucan as presently defined can characterize (i) an alpha-1,3-glucan side chain herein and (ii) an alpha-1,3-glucan used to prepare an ether or ester derivative, which is consequently used as a backbone in a graft copolymer. In some aspects, alpha-1,3-glucan can characterize an alpha-1,3-glucan “homopolymer”, which is alpha-1,3-glucan that is not part of (i) a graft copolymer or (ii) part of a [non-derivatized alpha-1,3-glucan]-[alpha-1,3-glucan ether/ester derivative) linear copolymer.

An alpha-1,3-glucan used to prepare an ether or ester derivative herein typically is linear (no branches) or substantially linear. “Completely linear” alpha-1,3-glucan has no branches, before being ether-or ester-derivatized and consequently used to produce a graft copolymer. A “substantially linear” alpha-1,3-glucan herein has 5% or less branches (e.g., alpha-1,2,-1,4, and/or-1,6), before being ether-or ester-derivatized and consequently used to produce a graft copolymer. Branches of a substantially linear alpha-1,3-glucan herein typically are short, being one (pendant) to three glucose monomers in length, and comprise less than about 5% of all the glucose monomers of the entire glucan molecule.

An alpha-1,3-glucan ether or ester derivative used in a glucosyltransferase reaction herein for alpha-1,3-glucan synthesis (thereby producing a copolymer) can optionally be characterized as a “primer”, “acceptor”, or other like term.

An “alpha-1,2 branch” (and like terms) herein comprises a glucose that is alpha-1,2-linked to an alpha-1,3-glucan; an alpha-1,2 branch herein can also be referred to as an alpha-1,2,3 linkage. An “alpha-1,6 branch” (and like terms) as referred to herein comprises a glucose that is alpha-1,6-linked to an alpha-1,3-glucan; an alpha-1,6 branch herein can also be referred to as an alpha-1,6,3 linkage. An “alpha-1,4 branch” (and like terms) as referred to herein comprises a glucose that is alpha-1,4-linked to an alpha-1,3-glucan; an alpha-1,4 branch herein can also be referred to as an alpha-1,4,3 linkage.

The percent branching in an alpha-glucan or graft copolymer herein refers to that percentage of all the glycosidic linkages therein that represent branch points.

The terms “glycosidic linkage”, “glycosidic bond” and the like refer to the covalent bonds connecting the sugar monomers within a saccharide compound (oligosaccharides and/or polysaccharides). Examples of glycosidic linkages include alpha-linked glucose oligomers with 1,6-alpha-D-glycosidic linkages (herein also referred to as “alpha-1,6” linkages); 1,3-alpha-D-glycosidic linkages (herein also referred to as “alpha-1,3” linkages); 1,4-alpha-D-glycosidic linkages (herein also referred to as “alpha-1,4” linkages); and 1,2-alpha-D-glycosidic linkages (herein also referred to as “alpha-1,2” linkages). The glycosidic linkages of a glucan polymer herein can also be referred to as “glucosidic linkages”. Herein, “alpha-D-glucose” is referred to as “glucose”. Alpha-1,2 linkages typically only occur at branch points, and do not occur in tandem (i.e., two or more consecutive glucose monomers are not joined by consecutive alpha-1,2 linkages).

The linkage profile of an alpha-glucan or graft copolymer herein can be determined using any method known in the art. For example, a linkage profile can be determined using methods using nuclear magnetic resonance (NMR) spectroscopy (e.g., ¹³C NMR or ¹H NMR). These and other methods that can be used are disclosed in, for example, Food Carbohydrates: Chemistry, Physical Properties, and Applications (S. W. Cui, Ed., Chapter 3, S. W. Cui, Structural Analysis of Polysaccharides, Taylor & Francis Group LLC, Boca Raton, FL, 2005), which is incorporated herein by reference.

The “molecular weight” of large alpha-glucan and graft copolymers herein can be represented as weight-average molecular weight (Mw) or number-average molecular weight (Mn), the units of which are in Daltons or grams/mole. Alternatively, such molecular weight can be represented as DPw (weight average degree of polymerization) or DPn (number average degree of polymerization). The molecular weight of smaller polymers such as oligosaccharides typically can be provided as “DP” ( degree of polymerization), which simply refers to the number of glucoses comprised within the alpha-glucan; “DP” can also characterize the molecular weight of a polymer on an individual molecule basis. Various means for calculating these various molecular weight measurements can be employed such as high-pressure liquid chromatography (HPLC), size exclusion chromatography (SEC), or gel permeation chromatography (GPC), and/or by following the procedure disclosed in the below Examples.

The term “sucrose” herein refers to a non-reducing disaccharide composed of an alpha-D-glucose molecule and a beta-D-fructose molecule linked by an alpha-1,2-glycosidic bond. Sucrose is known commonly as table sugar. Sucrose can alternatively be referred to as “alpha-D-glucopyranosyl-(1→2)-beta-D-fructofuranoside”. “Alpha-D-glucopyranosyl” and “glucosyl” are used interchangeably herein.

The terms “glucosyltransferase”, “glucosyltransferase enzyme”, “GTF”, “glucansucrase” and the like are used interchangeably herein. The activity of a glucosyltransferase herein catalyzes the reaction of the substrate sucrose to make the products alpha-glucan and fructose. Other products (by-products) of a GTF reaction can include glucose, various soluble gluco-oligosaccharides, and leucrose. Wild type forms of glucosyltransferase enzymes generally contain (in the N-terminal to C-terminal direction) a signal peptide (which is typically removed by cleavage processes), a variable domain, a catalytic domain, and a glucan-binding domain. A glucosyltransferase herein is classified under the glycoside hydrolase family 70 (GH70) according to the CAZy (Carbohydrate-Active EnZymes) database (Cantarel et al., Nucleic Acids Res. 37:D233-238, 2009).

The term “glucosyltransferase catalytic domain” herein refers to the domain of a glucosyltransferase enzyme that provides alpha-glucan-synthesizing activity to a glucosyltransferase enzyme. A glucosyltransferase catalytic domain typically does not require the presence of any other domains to have this activity.

The terms “enzymatic reaction”, “glucosyltransferase reaction”, “glucan synthesis reaction”, “reaction composition”, “reaction formulation” and the like are used interchangeably herein and generally refer to a reaction that initially comprises water, sucrose, at least one active glucosyltransferase enzyme, and optionally other components such as an alpha-1,3-glucan ether-or ester-derivative (as a primer). Components that can be further present in a glucosyltransferase reaction typically after it has commenced include fructose, glucose, leucrose, soluble gluco-oligosaccharides (e.g., DP2-DP7) (such may be considered as products or by-products, depending on the glucosyltransferase used), and/or insoluble alpha-glucan product(s) of DP8 or higher. It would be understood that certain glucan products, such as alpha-1,3-glucan with a degree of polymerization (DP) of at least 8 or 9, are water-insoluble and thus not dissolved in a glucan synthesis reaction, but rather may be present out of solution (e.g., by virtue of having precipitated from the reaction). It is in a glucan synthesis reaction where the step of contacting water, sucrose and a glucosyltransferase enzyme is performed. The term “under suitable reaction conditions” as used herein refers to reaction conditions that support conversion of sucrose to alpha-glucan product(s) via glucosyltransferase enzyme activity. It is during such a reaction that glucosyl groups originally derived from the input sucrose are enzymatically transferred and used in alpha-glucan polymer synthesis; glucosyl groups as involved in this process can thus optionally be referred to as the glucosyl component or moiety (or like terms) of a glucosyltransferase reaction.

The “yield” of insoluble alpha-glucan product in a glucosyltransferase reaction in some aspects herein represents the molar yield based on the converted sucrose. The molar yield of an alpha-glucan product can be calculated based on the moles of insoluble alpha-glucan product divided by the moles of the sucrose converted. Moles of converted sucrose can be calculated as follows: (mass of initial sucrose−mass of final sucrose)/molecular weight of sucrose [342 g/mol]. This molar yield calculation can be considered as a measure of selectivity of the reaction toward the alpha-glucan. In some aspects, the “yield” of insoluble alpha-glucan product in a glucosyltransferase reaction can be based on the glucosyl component of the reaction. Such a yield (yield based on glucosyl) can be measured using the following formula:

$$Insoluble Alpha - Glucan Yield = ( ( IS / 2 - ( FS / 2 + LE / 2 + GL + SO ) ) / ( IS / 2 - FS / 2 ) ) \times 100 \% .$$

The fructose balance of a glucosyltransferase reaction can be measured to ensure that HPLC data, if applicable, are not out of range (90-110% is considered acceptable). Fructose balance can be measured using the following formula:

$$Fructose Balance = ( ( 180 / 342 \times ( FS + LE ) + FR ) / ( 180 / 342 \times IS ) ) \times 100 \% .$$

In the above two formulae, IS is [Initial Sucrose], FS is [Final Sucrose], LE is [Leucrose], GL is [Glucose], SO is [Soluble Oligomers] (gluco-oligosaccharides), and FR is [Fructose]; the concentrations of each foregoing substrate/product provided in double brackets are in units of grams/L and as measured by HPLC, for example.

Terms used herein regarding “ethers” (e.g., alpha-1,3-glucan ether-derivative and like terms) are defined as in U.S. Patent Appl. Publ. Nos. 2014/179913, 2016/0304629, 2016/0311935, 2015/0239995, 2018/0230241, and/or 2018/0237816, which are incorporated herein by reference. An alpha-1,3-glucan ether compound has a DoS of about 0.001 to about 3.0 with one or more different types of organic group(s). An alpha-1,3-glucan ether compound is termed an “ether” herein by virtue of comprising the substructure —C_{G}—O—C—, where “—C_{G}—” represents carbon 2, 4, or 6 of a glucose monomeric unit of the alpha-1,3-glucan ether compound, and where “—C—” is comprised in the organic group.

An “organic group” group (e.g., an ether-linked organic group) in some aspects refers to a chain of one or more carbons that (i) has the formula —CₙH₂ₙ₊₁ (i.e., an alkyl group, which is completely saturated) or (ii) is mostly saturated but has one or more hydrogens substituted with another atom or functional group (i.e., a “substituted alkyl group”). Such substitution can be with one or more hydroxyl groups, oxygen atoms (thereby forming an aldehyde or ketone group), carboxyl groups, or other alkyl groups. A “hydroxy alkyl” group herein refers to a substituted alkyl group in which one or more hydrogen atoms of the alkyl group are substituted with a hydroxyl group. A “carboxy alkyl” group herein refers to a substituted alkyl group in which one or more hydrogen atoms of the alkyl group are substituted with a carboxyl group.

Terms used herein regarding “esters” (e.g., alpha-1,3-glucan ester-derivative and like terms) are defined as in U.S. Pat. Appl. Publ. Nos. 2014/0187767 and/or 2018/0155455, and/or Int. Patent Appl. Publ. No. WO2018/098065, which are incorporated herein by reference. An alpha-1,3-glucan ester compound herein has a DoS of about 0.001 to about 3.0 with one or more different types of acyl group(s). An alpha-1,3-glucan ester compound is termed an “ester” herein by virtue of comprising the substructure —C_{G}—O—CO—C—, where “—C_{G}—” represents carbon 2, 4, or 6 of a glucose monomeric unit of the alpha-1,3-glucan ester compound, and where “—CO—C—” is comprised in the acyl group.

The term “degree of substitution” (DOS) as used herein refers to the average number of hydroxyl groups that are substituted (with organic groups via ether linkage, or with acyl groups via ester linkage) in each monomeric unit (glucose) of an alpha-1,3-glucan ether-or ester-derivative herein. DoS herein specifically refers to substitution with organic groups or acyl groups, and does not refer to any substituting saccharide groups (alpha-1,3-glucan side chain) (the DoS of an alpha-1,3-glucan ether or ester backbone of a graft copolymer herein is strictly based on its substitution with organic or acyl groups). In this sense, it can be said that the DoS of an alpha-1,3-glucan ether-or ester-derivative is the same both before and after its use as a primer for alpha-1,3-glucan side arm synthesis.

The terms “percent by volume”, “volume percent”, “vol %”, “v/v %” and the like are used interchangeably herein. The percent by volume of a solute in a solution can be determined using the formula: [(volume of solute)/(volume of solution)]×100%.

The terms “percent by weight”, “weight percentage (wt %)”, “weight-weight percentage (% w/w)” and the like are used interchangeably herein. Percent by weight refers to the percentage of a material on a mass basis as it is comprised in a composition, mixture, or solution.

The term “weight/volume percent”, “w/v %” and the like are used interchangeably herein. Weight/volume percent can be calculated as: ((mass [g] of material)/(total volume [mL] of the material plus the liquid in which the material is placed))×100%. The material can be insoluble in the liquid (i.e., be a solid phase in a liquid phase, such as with a dispersion), or soluble in the liquid (i.e., be a solute dissolved in the liquid).

The terms “aqueous liquid”, “aqueous fluid” and the like as used herein can refer to water or an aqueous solution. An “aqueous solution” herein can comprise one or more dissolved salts, where the maximal total salt concentration can be about 3.5 wt % in some embodiments. Although aqueous liquids herein typically comprise water as the only solvent in the liquid, an aqueous liquid can optionally comprise one or more other solvents (e.g., polar organic solvent) that are miscible in water. Thus, an aqueous solution can comprise a solvent having at least about 10 wt % water.

An “aqueous composition” herein has a liquid component that comprises at least about 10 wt % water, for example. Examples of aqueous compositions include mixtures, solutions, dispersions (e.g., colloidal dispersions), suspensions and emulsions, for example.

As used herein, the term “colloidal dispersion” refers to a heterogeneous system having a dispersed phase and a dispersion medium, i.e., microscopically dispersed insoluble particles are suspended throughout another substance (e.g., an aqueous composition such as water or aqueous solution). An example of a colloidal dispersion herein is a hydrocolloid. All, or a portion of, the particles of a colloidal dispersion such as a hydrocolloid can comprise a graft copolymer herein. The terms “dispersant” and “dispersion agent” are used interchangeably herein to refer to a material that promotes the formation and/or stabilization of a dispersion. “Dispersing” herein refers to the act of preparing a dispersion of a material in an aqueous liquid.

An alpha-glucan or graft copolymer that is “insoluble”, “aqueous-insoluble”, “water-insoluble” (and like terms) (e.g., alpha-1,3-glucan with a DP of 8 or higher) does not dissolve (or does not appreciably dissolve) in water or other aqueous conditions, optionally where the aqueous conditions are further characterized to have a pH of 4-9 (e.g., pH 6-8) (i.e., non-caustic) and/or a temperature of about 1 to 85° C. (e.g., 20-25° C.). In contrast, alpha-glucans such as certain oligosaccharides herein that are “soluble”, “aqueous-soluble”, “water-soluble” and the like (e.g., alpha-1,3-glucan with a DP less than 8) appreciably dissolve under these conditions.

The term “viscosity” as used herein refers to the measure of the extent to which a fluid (aqueous or non-aqueous) resists a force tending to cause it to flow. Various units of viscosity that can be used herein include centipoise (cP, cps) and Pascal-second (Pa·s), for example. A centipoise is one one-hundredth of a poise; one poise is equal to 0.100 kg·m⁻¹·s⁻¹.

The term “zeta potential” as used herein refers to the electrical potential difference between a dispersion medium and the stationary layer of fluid attached to a graft copolymer particle dispersed in the dispersion medium. In general, a dispersed graft copolymer herein with a high zeta potential (negative or positive) is more electrically stabilized compared to a dispersed material with low zeta potentials (closer to zero). Since the repulsive forces of a high zeta potential material in a dispersion tend to exceed its attractive forces, such a dispersion is relatively more stable than a dispersion of low zeta potential material, which tends to more easily flocculate/coagulate.

The terms “particle”, “particulate” and other like terms are interchangeably used herein. “Particle size” (and like terms) in some aspects can refer to particle diameter and/or the length of the longest particle dimension. Average particle size can be based on the average of diameters and/or longest particle dimensions of at least about 50, 100, 500, 1000, 2500, 5000, or 10000 or more particles, for example.

The term “household care product” and like terms typically refer to products, goods and services relating to the treatment, cleaning, caring and/or conditioning of a home and its contents. The foregoing include, for example, chemicals, compositions, products, or combinations thereof having application in such care.

The terms “fabric”, “textile”, “cloth” and the like are used interchangeably herein to refer to a woven material having a network of natural and/or artificial fibers. Such fibers can be in the form of thread or yarn, for example.

A “fabric care composition” and like terms refer to any composition suitable for treating fabric in some manner. Examples of such a composition include laundry detergents and fabric softeners, which are examples of laundry care compositions.

The terms “heavy duty detergent”, “all-purpose detergent” and the like are used interchangeably herein to refer to a detergent useful for regular washing of white and/or colored textiles at any temperature. The terms “low duty detergent”, “fine fabric detergent” and the like are used interchangeably herein to refer to a detergent useful for the care of delicate fabrics such as viscose, wool, silk, microfiber or other fabric requiring special care. “Special care” can include conditions of using excess water, low agitation, and/or no bleach, for example.

A “detergent composition” herein typically comprises at least a surfactant (detergent compound) and/or a builder. A “surfactant” herein refers to a substance that tends to reduce the surface tension of a liquid in which the substance is dissolved. A surfactant may act as a detergent, wetting agent, emulsifier, foaming agent, and/or dispersant, for example.

The term “personal care product” and like terms typically refer to products, goods and services relating to the treatment, cleaning, cleansing, caring or conditioning of a person. The foregoing include, for example, chemicals, compositions, products, or combinations thereof having application in such care.

An “oral care composition” herein is any composition suitable for treating a soft or hard surface in the oral cavity such as dental (teeth) and/or gum surfaces.

The terms “sequence identity”, “identity” and the like as used herein with respect to a polypeptide amino acid sequence are as defined and determined in U.S. Patent Appl. Publ. No. 2017/0002336, which is incorporated herein by reference.

Various polypeptide amino acid sequences and polynucleotide sequences are disclosed herein as features of certain embodiments. Variants of these sequences that are at least about 70-85%, 85-90%, or 90%-95% identical to the sequences disclosed herein can be used or referenced. Alternatively, a variant amino acid sequence or polynucleotide sequence can have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity with a sequence disclosed herein. The variant amino acid sequence or polynucleotide sequence has the same function/activity of the disclosed sequence, or at least about 80%, 81%, 82%, 83%, 84%, 85%, 86%, 87%, 88%, 89%, 90%, 91%, 92%, 93%, 94%, 95%, 96%, 97%, 98%, or 99% of the function/activity of the disclosed sequence. Any polypeptide amino acid sequence disclosed herein not beginning with a methionine can typically further comprise at least a start-methionine at the N-terminus of the amino acid sequence. In contrast, any polypeptide amino acid sequence disclosed herein beginning with a methionine can optionally lack such a methionine residue.

The terms “aligns with”, “corresponds with”, and the like can be used interchangeably herein. Some embodiments herein relate to a glucosyltransferase comprising at least one amino acid substitution at a position corresponding with at least one particular amino acid residue of SEQ ID NO:62. An amino acid position of a glucosyltransferase or subsequence thereof (e.g., catalytic domain or catalytic domain plus glucan-binding domains) (can refer to such an amino acid position or sequence as a “query” position or sequence) can be characterized to correspond with a particular amino acid residue of SEQ ID NO:62 (can refer to such an amino acid position or sequence as a “subject” position or sequence) if (1) the query sequence can be aligned with the subject sequence (e.g., where an alignment indicates that the query sequence and the subject sequence [or a subsequence of the subject sequence] are at least about 30%, 40%, 50%, 60%, 70%, 80%, or 90% identical), and (2) if the query amino acid position directly aligns with (directly lines up against) the subject amino acid position in the alignment of (1). In general, one can align a query amino acid sequence with a subject sequence (SEQ ID NO:62 or a subsequence of SEQ ID NO:62) using any alignment algorithm, tool and/or software described disclosed herein (e.g., BLASTP, ClustalW, ClustalV, Clustal-Omega, EMBOSS) to determine percent identity. Just for further example, one can align a query sequence with a subject sequence herein using the Needleman-Wunsch algorithm (Needleman and Wunsch, J. Mol. Biol. 48:443-453, 1970) as implemented in the Needle program of the European Molecular Biology Open Software Suite (EMBOSS [e.g., version 5.0.0 or later], Rice et al., Trends Genet. 16:276-277, 2000). The parameters of such an EMBOSS alignment can comprise, for example: gap open penalty of 10, gap extension penalty of 0.5, EBLOSUM62 (EMBOSS version of BLOSUM62) substitution matrix.

The numbering of particular amino acid residues of SEQ ID NO:62 herein is with respect to the full-length amino acid sequence of SEQ ID NO:62. The first amino acid (i.e., position 1, Met-1) of SEQ ID NO:62 is at the start of the signal peptide. Unless otherwise disclosed, substitutions herein are with respect to the full-length amino acid sequence of SEQ ID NO:62.

A “non-native glucosyltransferase” herein (“mutant”, “variant”, “modified” and like terms can likewise be used to describe such a glucosyltransferase) has at least one amino acid substitution at a position corresponding with a particular amino acid residue of SEQ ID NO:62. Such at least one amino acid substitution typically is in place of the amino acid residue(s) that normally (natively) occurs at the same position in the native counterpart (parent) of the non-native glucosyltransferase (i.e., although SEQ ID NO:62 is used as a reference for position, an amino acid substitution herein is with respect to the native counterpart of a non-native glucosyltransferase) (considered another way, when aligning the sequence of a non-native glucosyltransferase with SEQ ID NO:62, determining whether a substitution exists at a particular position does not depend in-and-of-itself on the respective amino acid residue in SEQ ID NO:62, but rather depends on what amino acid exists at the subject position within the native counterpart of the non-native glucosyltransferase). The amino acid normally occurring at the relevant site in the native counterpart glucosyltransferase often (but not always) is the same as (or conserved with) the particular amino acid residue of SEQ ID NO:62 for which the alignment is made. A non-native glucosyltransferase optionally can have other amino acid changes (mutations, deletions, and/or insertions) relative to its native counterpart sequence.

The term “isolated” means a substance (or process) in a form or environment that does not occur in nature. A non-limiting example of an isolated substance includes any non-naturally occurring substance such as a graft copolymer herein (as well as the enzymatic reactions and other processes used in preparation thereof). It is believed that the embodiments disclosed herein are synthetic/man-made (could not have been made except for human intervention/involvement), and/or have properties that are not naturally occurring.

The term “increased” as used herein can refer to a quantity or activity that is at least about 1%, 2%, 3%, 4%, 5%, 6%, 7%, 8%, 9%, 10%, 11%, 12%, 13%, 14%, 15%, 16%, 17%, 18%, 19%, 20%, 50%, 100%, or 200% more than the quantity or activity for which the increased quantity or activity is being compared. The terms “increased”, “elevated”, “enhanced”, “greater than”, “improved” and the like are used interchangeably herein.

New forms of alpha-1,3-glucan are desired to enhance the economic value and performance characteristics of this material in various applications. Compositions comprising alpha-1,3-glucan in the form of a graft copolymer are presently disclosed to address this need.

Certain embodiments of the present disclosure concern a composition comprising a graft copolymer that comprises:
(i) a backbone comprising an alpha-1,3-glucan ether or ester compound that has a degree of substitution (DoS) of about 0.001 to about 3.0, and
(ii) one or more alpha-1,3-glucan side chains comprising at least about 50% alpha-1,3 glycosidic linkages.
   Significantly, graft copolymers of the present disclosure have several advantageous properties, including enhanced dispersion functions (e.g., viscosity and stability).

A graft copolymer herein has a backbone comprising an alpha-1,3-glucan ether compound or ester compound that has a DoS of about 0.001 to about 3.0. An alpha-1,3-glucan ether or ester compound can comprise about, or at least about, 50%, 60%, 70%, 80%, 85%, 90%, 95%, 96%, 97%, 98%, 99%, 99.5%, or 100% alpha-1,3 glycosidic linkages, for example. In some aspects, accordingly, there can be less than about 50%, 40%, 30%, 20%, 15%, 10%, 9%, 8%, 7%, 6%, 5%, 4%, 3%, 2%, 1%, 0.5%, or 0% glycosidic linkages that are not alpha-1,3. Typically, the glycosidic linkages that are not alpha-1,3 are mostly or entirely alpha-1,6. In certain embodiments, an alpha-1,3-glucan ether-or ester-derivative has no glycosidic branch points or less than about 5%, 4%, 3%, 2%, or 1% glycosidic branch points as a percent of the glycosidic linkages in the derivative. In aspects in which a backbone comprises 50% alpha-1,3 glycosidic linkages, such a backbone typically does not comprise alternan (alternating alpha-1,3 and-1,6 linkages).

An alpha-1,3-glucan ether compound or ester compound of a graft copolymer backbone herein can have a weight-average degree of polymerization (DPw) of about, at least about, or less than about, 11, 12, 15, 20, 25, 30, 35, 40, 45, 50, 55, 60, 65, 70, 75, 80, 85, 90, 95, 100, 150, 200, 300, 400, 500, 600, 700, 800, 900, 1000, 1100, 1200, 1250, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, or 4000, for example. DPw can optionally be expressed as a range between any two of these values. Merely as examples, the DPw can be about 11-4000, 15-4000, 11-1300, 15-1300, 400-1300, 500-1300, 600-1300, 700-1300, 400-1200, 500-1200, 600-1200, 700-1200, 400-1000, 500-1000, 600-1000, 700-1000, 400-900, 500-900, 600-900, 700-900, 11-25, 12-25, 11-22, 12-22, 11-20, 12-20, 20-300, 20-200, 20-150, 20-100, 20-75, 30-300, 30-200, 30-150, 30-100, 30-75, 50-300, 50-200, 50-150, 50-100, 50-75, 75-300, 75-200, 75-150, 75-100, 100-300, 100-200, 100-150, 150-300, 150-200, or 200-300.

Any of the preceding linkage and/or molecular weight features of an alpha-1,3-glucan ether-or ester-derivative can likewise characterize an alpha-1,3-glucan polymer used to prepare the alpha-1,3-glucan ether-or ester-derivative. Any glucosyltransferase disclosed herein for synthesizing alpha-1,3-glucan side chains comprising at least about 50% alpha-1,3 linkages can be used to synthesize alpha-1,3-glucan for ether-or ester-derivatization.

A backbone of a graft copolymer in some aspects can be comprised entirely of an alpha-1,3-glucan ether-or ester-derivative as presently disclosed. However, in some aspects, a backbone can comprise other elements. For example, a graft copolymer backbone can comprise non-derivatized alpha-1,3-glucan originating from a/the non-reducing end of an alpha-1,3-glucan ether-or ester-derivative, by virtue of the glucan derivative (at a non-reducing end) serving to prime alpha-1,3-glucan synthesis during synthesis of the graft copolymer (during the step of adding alpha-1,3-glucan side chains). In such aspects, the DoS of the backbone can be considered to be the DoS of the alpha-1,3-glucan derivative as it existed before the addition of non-derivatized alpha-1,3-glucan during side chain synthesis.

In some alternative aspects, an alpha-1,3-glucan ether compound or ester compound of a graft copolymer backbone herein can comprise at least about 30% alpha-1,3 linkages and a percentage of alpha-1,6 linkages that brings the total of both the alpha-1,3 and-1,6 linkages in the backbone to 100%. For example, the percentage of alpha-1,3 and -1,6 linkages can be about 30-40% and 60-70%, respectively. Glucosyltransferases contemplated to be useful for producing alpha-1,3-glucan for preparing such derivatives are disclosed in U.S. Patent Appl. Publ. No. 2015/0232819, which is incorporated herein by reference.

In some alternative aspects, a graft copolymer herein can have, as a backbone, an ether-or ester-derivative of another type of alpha-glucan, such as dextran (alpha-glucan with at least 90%, 95%, 99% or 100% alpha-1,6 linkages), alpha-1,4-glucan (e.g., with at least 90%, 95%, 99% or 100% alpha-1,4 linkages), alternan, or reuteran. Any of the above DPw values/ranges can apply to any of these types of backbones.

The backbone of a graft copolymer in some aspects can comprise an alpha-1,3-glucan ether compound. The DoS of an alpha-1,3-glucan ether with one or more etherified organic groups can be about 0.001 to about 3.0, for example. The DoS in some aspects can be about, or at least about, or up to about, 0.001, 0.0025, 0.005, 0.01, 0.025, 0.05, 0.075, 0.1, 0.2, 0.25, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 (DoS can optionally be expressed as a range between any two of these values). Examples of DoS ranges herein include 0.05-2.6, 0.05-2.4, 0.05-2.2, 0.05-2.0, 0.05-1.8, 0.05-1.6, 0.05-1.4, 0.05-1.3, 0.05-1.2, 0.05-1.1, 0.05-1.0, 0.05-0.8, 0.05-0.6, 0.05-0.4, 0.1-2.6, 0.1-2.4, 0.1-2.2, 0.1-2.0, 0.1-1.8, 0.1-1.6, 0.1-1.4, 0.1-1.3, 0.1-1.2, 0.1-1.1, 0.1-1.0, 0.1-0.8, 0.1-0.6, 0.1-0.4, 0.2-2.6, 0.2-2.4, 0.2-2.2, 0.2-2.0, 0.2-1.8, 0.2-1.6, 0.2-1.4, 0.2-1.3, 0.2-1.2, 0.2-1.1, 0.2-1.0, 0.2-0.8, 0.2-0.6, 0.2-0.4, 0.3-2.6, 0.3-2.4, 0.3-2.2, 0.3-2.0, 0.3-1.8, 0.3-1.6, 0.3-1.4, 0.3-1.3, 0.3-1.2, 0.3-1.1, 0.3-1.0, 0.3-0.8, 0.3-0.6, and 0.3-0.4. An ether group can be anionic, uncharged (nonionic), or cationic; the charge of an ether group can be as it exists when the graft copolymer ether derivative is in an aqueous composition herein, for example, further taking into account the pH of the aqueous composition (in some aspects, the pH can be 4-10 or 5-9).

An organic group etherified to an alpha-1,3-glucan backbone of a graft copolymer herein can be, for example, any of those as disclosed in U.S. Patent Appl. Publ. Nos. 2014/179913, 2016/0304629, 2016/0311935, 2015/0232785, 2015/0239995, 2018/0237816, and 2019/0202942, and Int. Patent Appl. Publ. Nos. WO2017/218389 and WO2017/218391, which are incorporated herein by reference. An organic group etherified to an alpha-1,3-glucan backbone of a graft copolymer herein can comprise an alkyl group such as a methyl, ethyl, propyl, butyl, pentyl, hexyl, heptyl, octyl, nonyl, or decyl group, for example (these are examples of uncharged groups). In some aspects, an organic group can be a substituted alkyl group in which there is a substitution on one or more carbons of the alkyl group. The substitution(s) may be one or more hydroxyl, aldehyde, ketone, and/or carboxyl groups. For example, a substituted alkyl group can be a hydroxy alkyl group, dihydroxy alkyl group, or carboxy alkyl group. Examples of suitable hydroxy alkyl groups include hydroxymethyl, hydroxyethyl, hydroxypropyl, hydroxybutyl and hydroxypentyl groups (these are examples of uncharged groups). Other examples include dihydroxy alkyl groups (diols) such as dihydroxymethyl, dihydroxyethyl, dihydroxypropyl, dihydroxybutyl and dihydroxypentyl groups (these are examples of uncharged groups). Examples of suitable carboxy alkyl groups include carboxymethyl (—CH₂COOH), carboxyethyl, carboxypropyl, carboxybutyl and carboxypentyl groups (these are examples of anionic groups). An organic group in some aspects can comprise an aryl group such as a benzyl group.

An organic group etherified to an alpha-1,3-glucan backbone of a graft copolymer herein can be a positively charged (cationic) organic group in some aspects. A positively charged group can be, for example, any of those as disclosed in U.S. Patent Appl. Publ. No. 2016/0311935, which is incorporated herein by reference. A positively charged group can comprise a substituted ammonium group, for example. Examples of substituted ammonium groups are primary, secondary, tertiary and quaternary ammonium groups. An ammonium group can be substituted with one, two, or three alkyl groups (e.g., methyl, ethyl, propyl, butyl, pentyl, hexyl, heptyl, octyl, nonyl, decyl), for example. One of the groups of a substituted ammonium group comprises one carbon, or a chain of carbons, in ether linkage to alpha-1,3-glucan; such a carbon or carbon chain can be —CH₂—, —CH₂CH—, —CH₂CH₂CH₂—, —CH₂CH₂CH₂CH₂—, or —CH₂CH₂CH₂CH₂CH₂—, for example. A carbon or carbon chain in this context can optionally have at least one substitution with an oxygen atom (e.g., alcohol group) and/or alkyl group (e.g., methyl, ethyl, propyl, butyl). One or more positively charged organic groups in some aspects can be trimethylammonium hydroxypropyl groups (structure I, when each of R₂, R₃ and R₄ is a methyl group).

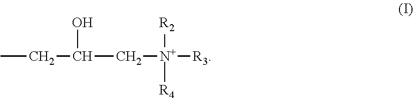

An alpha-1,3-glucan ether backbone of a graft copolymer in certain aspects can contain one type of etherified organic group. Non-limiting examples of such a backbone is carboxymethyl alpha-1,3-glucan or benzyl alpha-1,3-glucan. Alternatively, an alpha-1,3-glucan ether backbone can contain two or more different types of etherified organic groups (e.g., combinations of [i] carboxymethyl and benzyl groups, [ii] hydroxypropyl and hydroxyethyl groups, [iii] hydroxypropyl and ethyl groups, [iv] propyl and ethyl groups, or [v] propyl and hydroxyethyl groups). In some aspects, an alpha-1,3-glucan ether backbone can comprise at least one nonionic organic group and at least one anionic group as ether groups. In some aspects, an alpha-1,3-glucan ether backbone can comprise at least one nonionic organic group and at least one positively charged organic group as ether groups. Thus, an alpha-1,3-glucan ether backbone herein can optionally be amphiphilic.

Any suitable process for ether-derivatizing polysaccharides can be employed to prepare an alpha-1,3-glucan ether backbone for graft copolymer production, such as disclosed in U.S. Pat. Nos. 2,961,439, 2,344,179, 2,203,703, 2,203,704, 2,380,879 and 2,974,134, U.S. Patent Appl. Publ. Nos. 2014/179913, 2016/0304629, 2016/0311935, 2015/0232785, 2015/0239995, 2018/0237816, and 2019/0202942, and Int. Patent Appl. Publ. Nos. WO2017/218389 and WO2017/218391, all of which are incorporated herein by reference.

The backbone of a graft copolymer in some aspects can comprise an alpha-1,3-glucan ester compound. The DoS of an alpha-1,3-glucan ester with one or more acyl groups can be about 0.001 to about 3.0, for example. The DoS with an acyl group in some aspects can have the same value or range as listed above for ether derivatives.

An acyl group esterified to an alpha-1,3-glucan backbone of a graft copolymer herein can be, for example, any of those as disclosed in U.S. Patent Appl. Publ. Nos. 2014/0187767 and 2018/0155455, and Int. Patent Appl. Publ. No. WO2018/098065, which are incorporated herein by reference. Examples of acyl groups herein include methanoyl (formyl), ethanoyl (acetyl, —CO—CH₃), propanoyl (propionyl), butanoyl (butyryl), pentanoyl (valeryl), hexanoyl (caproyl), heptanoyl (enanthyl), octanoyl (caprylyl), nonanoyl (pelargonyl), decanoyl (capryl), undecanoyl, dodecanoyl (lauroyl), tridecanoyl, tetradecanoyl (myristyl), pentadecanoyl, hexadecanoyl (palmityl), heptadecanoyl, octadecanoyl (stearyl), nonadecanoyl, eicosanoyl (arachidyl), uneicosanoyl, docosanoyl (behenyl), tricosanoyl, tetracosanoyl (lignoceryl), pentacosanoyl and hexacosanoyl (cerotyl) groups, for example. Additional examples of acyl groups herein include branched acyl groups (e.g., 2-methylpropanoyl, 2-methylbutanoyl, 2,2-dimethylpropanoyl, 3-methylbutanoyl, 2-methylpentanoyl, 3-methylpentanoyl group, 4-methylpentanoyl, 2,2-dimethylbutanoyl, 2,3-dimethylbutanoyl, 3,3-dimethylbutanoyl group, 2-ethylbutanoyl group, 2-ethylhexanoyl), cyclic acyl groups (e.g., cyclopropanoyl, cyclobutanoyl, cyclopentanoyl, cyclohexanoyl, cycloheptanoyl), and aryl acyl groups (e.g., benzoyl). Additional examples of acyl groups herein include —CO—CH₂—CH₂—COOH, —CO—CH₂—CH₂—CH₂—COOH, —CO—CH₂—CH₂—CH₂—CH₂—COOH, —CO—CH₂—CH₂—CH₂—CH₂—CH₂—COOH, —CO—CH₂—CH₂—CH₂—CH₂—CH₂—CH₂—COOH, —CO—CH═CH—COOH, —CO—CH═CH—CH₂—COOH, —CO—CH═CH—CH₂—CH₂—COOH, —CO—CH═CH—CH₂—CH₂—CH₂—COOH, —CO—CH═CH—CH₂—CH₂—CH₂—CH₂—COOH, —CO—CH₂—CH═CH—COOH, —CO—CH₂—CH═CH—CH₂—COOH, —CO—CH₂—CH═CH—CH₂—CH₂—COOH, —CO—CH₂—CH═CH—CH₂—CH₂—CH₂—COOH, —CO—CH₂—CH₂—CH═CH—COOH, —CO—CH₂—CH₂—CH═CH—CH₂—COOH, —CO—CH₂—CH₂—CH═CH—CH₂—CH₂—COOH, —CO—CH₂—CH₂—CH₂—CH═CH—COOH, —CO—CH₂—CH₂—CH₂—CH═CH—CH₂—COOH, —CO—CH₂—CH₂—CH₂—CH₂—CH═CH—COOH,

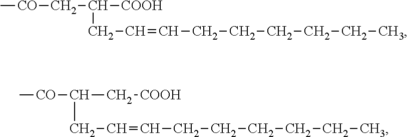 [SMILES: CCCCCCC=CCC(CC(C)=O)C(=O)O ; CCCCCCC=CCC(CC(=O)O)C(C)=O]

and any other acyl group that can be formed using a cyclic organic anhydride as an ester-derivatization agent.

An alpha-1,3-glucan ester backbone of a graft copolymer in certain aspects can contain one type of acyl group (e.g., acetyl group or benzoyl group). Alternatively, a graft copolymer ester compound can contain two or more different types of acyl groups (e.g., combinations of two of three of acetyl, propionyl, and/or butyryl groups).

Any suitable process for ester-derivatizing polysaccharides can be employed to prepare an alpha-1,3-glucan ester backbone for graft copolymer production, such as disclosed in U.S. Patent Appl. Publ. Nos. 2014/0187767 and 2018/0155455, and Int. Patent Appl. Publ. No. WO2018/098065, which are incorporated herein by reference.

A graft copolymer as presently disclosed comprises one or more alpha-1,3-glucan side chains comprising at least about 50% alpha-1,3 glycosidic linkages. An alpha-1,3-glucan side chain in certain aspects can comprise about, or at least about, 50%, 60%, 70%, 80%, 85%, 90%, 95%, 96%, 97%, 98%, 99%, 99.5%, or 100% alpha-1,3 glycosidic linkages. In some aspects, accordingly, an alpha-1,3-glucan side chain has less than about 50%, 40%, 30%, 20%, 15%, 10%, 9%, 8%, 7%, 6%, 5%, 4%, 3%, 2%, 1%, 0.5%, or 0% glycosidic linkages that are not alpha-1,3. Typically, the glycosidic linkages that are not alpha-1,3 are mostly or entirely alpha-1,6. In certain embodiments, an alpha-1,3-glucan side chain has no branch points or less than about 5%, 4%, 3%, 2%, or 1% branch points as a percent of the glycosidic linkages in the side chain. Glucosyltransferases contemplated to be useful for producing alpha-1,3-glucan side chains comprising at least about 50% alpha-1,3 linkages as above are disclosed herein and in U.S. Pat. Nos. 7,000,000 and 8,871,474, and Int. Patent Appl. Publ. No. WO2017/079595, all of which are incorporated herein by reference.

The DP of one or more alpha-1,3-glucan side chains in certain aspects can individually be about, or at least about, or less than about, 11, 12, 15, 20, 25, 30, 35, 40, 45, 50, 55, 60, 65, 70, 75, 80, 85, 90, 95, 100, 150, 200, 300, 400, 500, 600, 700, 800, 900, 1000, 1100, 1200, 1300, 1400, 1500, 1600, or 1650. DP can optionally be expressed as a range between any two of these values. Merely as examples, the DP of one or more alpha-1,3-glucan side chains can individually be about 400-1650, 500-1650, 600-1650, 700-1650, 400-1250, 500-1250, 600-1250, 700-1250, 400-1200, 500-1200, 600-1200, 700-1200, 400-1000, 500-1000, 600-1000, 700-1000, 400-900, 500-900, 600-900, 700-900, 11-25, 12-25, 11-22, 12-22, 11-20, 12-20, 20-300, 20-200, 20-150, 20-100, 20-75, 30-300, 30-200, 30-150, 30-100, 30-75, 50-300, 50-200, 50-150, 50-100, 50-75, 75-300, 75-200, 75-150, 75-100, 100-300, 100-200, 100-150, 150-300, 150-200, or 200-300. The DPw of a plurality of alpha-1,3-glucan side chains of a graft copolymer can be referred to, if desired; any of the foregoing DP values, which characterize side chains on an individual basis, can optionally be considered a DPw of all the side chains of a copolymer. In some aspects in which a graft copolymer has a plurality of alpha-1,3-glucan side chains, the individual DP values of the side chains are similar to each other (e.g., the DP values vary by less than 2.5%, 5%, 10%, 15%, or 20%).

In some aspects, an alpha-1,3-glucan side chain can comprise at least about 30% alpha-1,3 linkages and a percentage of alpha-1,6 linkages that brings the total of both the alpha-1,3 and-1,6 linkages in the side chain to 100%. For example, the percentage of alpha-1,3 and-1,6 linkages can be about 30-40% and 60-70%, respectively.

Glucosyltransferases contemplated to be useful for producing alpha-1,3-glucan side chains comprising at least about 30% alpha-1,3 linkages are disclosed in U.S. Patent Appl. Publ. No. 2015/0232819, which is incorporated herein by reference.

One or more alpha-1,3-glucan side chains in some aspects are contemplated to be (A) joined directly to a glucose unit of the backbone via alpha-glycosidic linkage (e.g., alpha-1,6, alpha-1,4, or alpha-1,2) (in some cases, such linkage might result from the promiscuous activity of an alpha-1,3-glucan-synthesizing glucosyltransferase enzyme), (B) extensions of pre-existing branches (e.g., alpha-1,2,-1,4, and/or-1,6), as such branches present non-reducing ends that can possibly prime alpha-1,3-glucan synthesis by a glucosyltransferase enzyme; and/or (C) possibly joined in another manner. In some aspects, alpha-1,3-glucan side chains are all linked to the backbone via the linkage type of (A) or (B), or via a combination of both (A) and (B) linkage types. Regarding the latter, a combination of both linkage types can comprise about 5%, 10%, 15%, 20%, 25%, 30%, 35%, 40%, 45%, 50%, 55%, 60%, 65%, 70%, 75%, 80%, 85%, 90%, or 95% of linkage type (A), with the balance of the other linkages being of type (B). The number of alpha-1,3-glucan side chains of a graft copolymer herein can be about, at least about, or up to about, 10%, 20%, 30%, 40%, 50%, 60%, 70%, 80%, or 90% of the number of monomeric units of the alpha-1,3-glucan derivative component of the graft copolymer. In some aspects, a graft copolymer is contemplated to comprise about, at least about, or less than about, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% branch points; such branchpoints can be those of alpha-1,3-glucan side arms (directly linked to backbone and/or linked via a pre-existing branch on the backbone) and/or branches not having alpha-1,3-glucan extended therefrom (e.g., alpha-1,2,-1,4, or -1,6 branch).

A graft copolymer as presently disclosed can be aqueous insoluble or aqueous soluble, but typically is aqueous insoluble. Graft copolymer insolubility can be under non-caustic aqueous conditions, such as the conditions of a glucosyltransferase reaction herein (e.g., pH 4-8, see below). In some aspects, a graft copolymer is insoluble in aqueous conditions at a temperature up to about 50, 60, 70, 80, 90, 100, 110, or 120° C. An aqueous composition herein such as an aqueous solution can comprise a solvent having about, or at least about, 10, 20, 30, 40, 50, 60, 70, 80, 90, or 100 wt % water, for example.

The DPw of a graft copolymer herein can be the sum of the DPw of any alpha-1,3-glucan ether- or ester-derivative backbone herein (such DPw can optionally be that of the alpha-1,3-glucan prior to its ether-or ester-derivatization) plus the DP/DPw of any alpha-1,3-glucan side chain(s) herein, for example. Merely as examples, the DPw of a graft copolymer herein can be about 1000-3000, 1500-3000, 2000-3000, 2500-3000, 1000-2500, 1500-2500, 2000-2500, 1000-2000, 1500-2000, or 1000-1500. Other examples include any of the DPw values disclosed in the below Examples for a graft copolymer product.

A graft copolymer herein can comprise about, or at least about, 1%, 5%, 10%, 15%, 20%, 25%, 30%, 35%, 40%, 45%, 50%, 55%, or 60% (e.g., by weight, or by mole percent [mol %]) (or a range between any two of these values) of alpha-1,3-glucan ether-or ester-derivative backbone, for example. In some aspects, a graft copolymer can comprise about 1-60%, 1-50%, 1-40%, 1-30%, 10-60%, 10-50%, 10-40%, 10-30%, 20-60%, 20-50%, 20-40%, or 20-30% (by wt % or mol %) of alpha-1,3-glucan ether-or ester-derivative backbone.

A graft copolymer as presently disclosed can be a product of any of the enzymatic reaction processes disclosed below, for example.

Certain embodiments of the present disclosure concern a method of producing (preparing) a graft copolymer as described herein. Such a graft polymer production method can comprise: (a) contacting (in the context of a reaction composition) at least (i) water, (ii) sucrose, (iii) an alpha-1,3-glucan ether or ester compound that has a DoS of about 0.001 to about 3.0 (serves as a primer), and (iv) a glucosyltransferase enzyme that synthesizes alpha-1,3-glucan comprising at least about 50% alpha-1,3 glycosidic linkages, thereby producing a graft copolymer as presently disclosed, optionally wherein the viscosity of the reaction composition increases by at least 10% at least 1 hour following the contacting step; and (b) optionally, isolating the graft copolymer produced in step (a). Step (a) can optionally be characterized as performing a reaction (or preparing/providing a reaction composition) comprising at least water, sucrose, an alpha-1,3-glucan ether or ester compound that has a DoS of about 0.001 to about 3.0, and a glucosyltransferase enzyme that synthesizes alpha-1,3-glucan with at least about 50% alpha-1,3 glycosidic linkages. A graft polymer production method herein can optionally further comprise, prior to step (a), ether-or ester-derivatizing alpha-1,3-glucan to provide an alpha-1,3-glucan ether or ester compound (for use in step [a]). Any feature of a graft polymer production method herein (e.g., features of a backbone, alpha-1,3-glucan side chains, graft copolymer) can be as described elsewhere herein. For example, the alpha-1,3-glucan derivative can be an ether (e.g., an anionic ether such as carboxymethyl ether) and/or comprise over 99% alpha-1,3 glycosidic linkages, and the side arms can comprise over 99% alpha-1,3 glycosidic linkages. A method herein of producing a graft copolymer can also be characterized as a method of producing alpha-1,3-glucan, if desired.

A glucosyltransferase enzyme for producing alpha-1,3-glucan side chains of a graft copolymer herein can be derived from any microbial source, such as bacteria. Examples of bacterial glucosyltransferase enzymes are those derived from a Streptococcus species, Leuconostoc species or Lactobacillus species. Examples of Streptococcus species include S. salivarius, S. sobrinus, S. dentirousetti, S. downei, S. mutans, S. oralis, S. gallolyticus and S. sanguinis. Examples of Leuconostoc species include L. mesenteroides, L. amelibiosum, L. argentinum, L. carnosum, L. citreum, L. cremoris, L. dextranicum and L. fructosum. Examples of Lactobacillus species include L. acidophilus, L. delbrueckii, L. helveticus, L. salivarius, L. casei, L. curvatus, L. plantarum, L. sakei, L. brevis, L. buchneri, L. fermentum and L. reuteri.

A glucosyltransferase enzyme for producing alpha-1,3-glucan side chains of a graft copolymer herein can in some aspects comprise an amino acid sequence as disclosed in any of U.S. Patent Appl. Publ. Nos. 2014/0087431, 2017/0166938, 2017/0002335 2018/0072998 and 2019/0078062 (corresponds to U.S. patent application Ser. No. 16/127,288), all of which are incorporated herein by reference. In some aspects, a glucosyltransferase enzyme herein can comprise an amino acid sequence that is 100% identical to, or at least 90%, 91%, 92%, 93%, 94%, 95%, 96%, 97%, 98%, 98.5%, 99%, or 99.5% identical to, SEQ ID NO:2, 4, 6, 8, 10, 12, 14, 16, 18, 20, 26, 28, 30, 34, or 59 (Table 1), and have glucosyltransferase activity. It is noted that a glucosyltransferase enzyme with SEQ ID NO:2, 4, 8, 10, 14, 20, 26, 28, 30, or 34 can synthesize alpha-1,3-glucan side chains comprising at least about 90% (˜100%) alpha-1,3 linkages.

The amino acid sequence of a glucosyltransferase enzyme in certain aspects has been modified such that the enzyme produces more products (alpha-1,3-glucan and fructose), and less by-products (e.g., glucose, oligosaccharides such as leucrose), from a given amount of sucrose substrate. For example, one, two, three, four, or more amino acid residues of the catalytic domain of a glucosyltransferase herein can be modified/substituted to obtain an enzyme that produces more products (insoluble alpha-glucan and fructose). Examples of a suitable modified glucosyltransferase enzyme are disclosed in the below Examples (Tables A and B). A modified glucosyltransferase enzyme, for example, can comprise one or more amino acid substitutions corresponding with those in Tables A and/or B (or in Table 3 of U.S. Patent Appl. Publ. No. 2018/0072998 [incorporated herein by reference, corresponding to application Ser. No. 15/702,893]) that is/are associated with an alpha-1,3-glucan yield of at least 40% (the position numbering of such at least one substitution corresponds with the position numbering of SEQ ID NO:62). A set of amino acid modifications as listed in Tables A or B can be used, for example.

The amino acid sequence of a glucosyltransferase enzyme in certain aspects has been modified such that the enzyme produces alpha-1,3-glucan with a molecular weight (DPw) that is lower than the molecular weight of alpha-1,3-glucan produced by its corresponding parent glucosyltransferase. Examples of a suitable modified glucosyltransferase enzyme are disclosed in the below Examples (Tables C and D). A modified glucosyltransferase enzyme, for example, can comprise one or more amino acid substitutions corresponding with those in Tables C and/or D that is/are associated with an alpha-1,3-glucan product molecular weight that is at least 5% less than the molecular weight of alpha-1,3-glucan produced by parent enzyme (the position numbering of such at least one substitution corresponds with the position numbering of SEQ ID NO:62). A set of amino acid modifications as listed in Table D can be used, for example.

The amino acid sequence of a glucosyltransferase enzyme in certain aspects has been modified such that the enzyme produces alpha-1,3-glucan with a molecular weight (DPw) that is higher than the molecular weight of alpha-1,3-glucan produced by its corresponding parent glucosyltransferase. Examples of a suitable modified glucosyltransferase enzyme are disclosed in the below Examples (Tables E and F). A modified glucosyltransferase enzyme, for example, can comprise one or more amino acid substitutions corresponding with those in Tables E and/or F that is/are associated with an alpha-1,3-glucan product molecular weight that is at least 5% higher than the molecular weight of alpha-1,3-glucan produced by parent enzyme (the position numbering of such at least one substitution corresponds with the position numbering of SEQ ID NO:62). A set of amino acid modifications as listed in Table 5 of U.S. Patent Appl. Publ. No. 2019/0078062 (incorporated herein by reference, corresponds to application Ser. No. 16/127,288) can be used, for example.

In some aspects, a modified glucosyltransferase (i) comprises at least one amino acid substitution or a set of amino acid substitutions (as described above regarding yield or molecular weight), and (ii) comprises or consists of a glucosyltransferase catalytic domain that is at least about 90%, 91%, 92%, 93%, 94%, 95%, 96%, 97%, 98%, 99%, or 99.5% identical to amino acid residues 55-960 of SEQ ID NO:4, residues 54-957 of SEQ ID NO:65, residues 55-960 of SEQ ID NO:30, residues 55-960 of SEQ ID NO:28, or residues 55-960 of SEQ ID NO:20. Each of these subsequences are the approximate catalytic domains of each respective reference sequence, and are believed to be able to produce alpha-1,3-glucan comprising at least about 50% (e.g., ≥90% or ≥95%) alpha-1,3 linkages, and optionally further have a DPw of at least 100. In some aspects, a modified glucosyltransferase (i) comprises at least one amino acid substitution or a set of amino acid substitutions (as described above), and (ii) comprises or consists of an amino acid sequence that is at least about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 69%, 70%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5% identical to SEQ ID NO:62 or a subsequence thereof such as SEQ ID NO:4 (without start methionine thereof) or positions 55-960 of SEQ ID NO:4 (approximate catalytic domain).

The temperature of a reaction composition herein can be controlled, if desired, and can be about 5-50° C., 20-40° C., 30-40° C., 20-30° C., 20-25° C., 20° C., 25° C., 30° C., 35° C., or 40° C., for example.

The initial concentration of sucrose in a reaction composition herein can be about, at least about, or less than about, 10, 15, 20, 25, 30, 40, 45, 50, 55, 60, 80, 90, 95, 100, 105, 110, 125, 150, 200, 300, 400, 500, 600, 10-50, 10-40, 10-30, 10-25, 15-50, 15-40, 15-30, or 15-25 g/L, or a range between any two of these values. Merely as examples, the initial sucrose concentration can be about 10-150, 40-60, 45-55, 90-110, or 95-105 g/L, for example. “Initial concentration of sucrose” refers to the sucrose concentration in a reaction composition just after all the reaction components have been added/combined (e.g., at least water, sucrose, an alpha-1,3-glucan ether or ester compound, glucosyltransferase enzyme).

The initial concentration of an alpha-1,3-glucan ether or ester compound as presently disclosed in a reaction composition can be about, or at least about, 0.1, 0.5, 1, 2, 2.5, 3, 4, 5, 6, 7, 8, 9, 10, 11, 12, 13, 14, 15, 16, 17, 18, 19, or 20 g/L, or a range between any two of these values. Merely as examples, the initial concentration of an alpha-1,3-glucan ether or ester compound can be about 0.1-15, 0.5-15, 1-15, 2-15, 4-15, 0.1-10, 0.5-10, 1-10, 2-10, 4-10, 0.1-5, 0.5-5, 1-5, 2-5, 4-5, 0.1-2.5, 0.5-2.5, 1-2.5, or 2-2.5 g/L. Any of the foregoing values (in g/L) can optionally rather be expressed in terms of w/v %. An alpha-1,3-glucan ether or ester compound typically is soluble in aqueous conditions as presently disclosed (e.g., reaction composition); such a compound is dissolved (along with sucrose, buffer and enzyme components) in setting up a reaction composition.

The pH of a reaction composition in certain embodiments can be about 4.0-9.0, 4.0-8.5, 4.0-8.0, 5.0-8.0, 5.5-7.5, or 5.5-6.5. In some aspects, the pH can be about 4.0, 4.5, 5.0, 5.5, 6.0, 6.5, 7.0, 7.5, or 8.0. The pH can be adjusted or controlled by the addition or incorporation of a suitable buffer, including but not limited to: phosphate, tris, citrate, or a combination thereof. The buffer concentration in a reaction composition herein can be about 0.1-300 mM, 0.1-100 mM, 10-100 mM, 5 mM, 10 mM, 20 mM, or 50 mM, for example.

A reaction composition can be contained within any vessel (e.g., an inert vessel/container) suitable for applying one or more of the reaction conditions disclosed herein. An inert vessel in some aspects can be of stainless steel, plastic, or glass (or comprise two or more of these components) and be of a size suitable to contain a particular reaction. For example, the volume/capacity of an inert vessel (and/or the volume of a reaction composition herein), can be about, or at least about, 1, 10, 50, 100, 500, 1000, 2500, 5000, 10000, 12500, 15000, or 20000 liters. An inert vessel can optionally be equipped with a stirring device. Any of the foregoing features, for example, can be used to characterize an isolated reaction herein.

A reaction composition herein can contain one, two, or more different glucosyltransferase enzymes that produce alpha-1,3-glucan side chains, for example. In some aspects, only one or two glucosyltransferase enzymes is/are comprised in a reaction composition. A reaction composition herein can be, and typically is, cell-free (e.g., no whole cells present).

Completion of a reaction in certain aspects can be determined visually (e.g., no more accumulation of insoluble graft copolymer product), and/or by measuring the amount of sucrose left in the solution (residual sucrose), where a percent sucrose consumption of at least about 90%, 95%, or 99% can indicate reaction completion. In some aspects, a reaction can be considered complete when its sucrose content is at or below about 2-5 g/L. A reaction of the disclosed process can be conducted for about, at least about, or up to about, 1, 1.5, 2, 2.5, 3, 3.5, 4, 6, 8, 10, 12, 14, 16, 18, 20, 22, 24, 36, 48, 60, 72, 96, 120, 144, 168, 1-4, 1-3.5, 1-3, 1.5-4, 1.5-3.5, 1.5-3, 2-4, 2-3.5, or 2-3 hours, for example. A reaction can optionally be terminated and/or otherwise treated to stop glucosyltransferase activity by heating it to at least about 65° C. for at least about 30-60 minutes.

Examples of other conditions and/or components suitable for synthesizing alpha-1,3-glucan side chains in graft copolymer production herein are disclosed in U.S. Patent Appl. Publ. Nos. 2014/0087431, 2017/0166938 and 2017/0002335, which are incorporated herein by reference.

Graft copolymer produced in a reaction composition herein can optionally be isolated. In certain embodiments, isolating graft copolymer can include at least conducting a step of centrifugation, filtration, fractionation, chromatographic separation, dialysis, evaporation, or dilution. Isolation of insoluble graft copolymer can include at least conducting a step of preparing a cake of graft copolymer. Cake preparation can include at least conducting a step of centrifugation (cake is pelleted graft copolymer) and/or filtration (cake is filtered graft copolymer). Isolation can optionally further comprise washing the centrifuged and/or filtered graft copolymer one, two, or more times with water or other aqueous liquid. A wash volume can optionally be at least about 10-100% of the volume of the reaction composition used to produce the graft copolymer. Washing can be done by various modes, as desired, such as by displacement or re-slurry washing. In some aspects, the aqueous portion of the resulting cake has no (detectable) dissolved sugars, or about 0.1-1.5, 0.1-1.25, 0.1-1.0, 0.1-0.75, 0.1-0.5, 0.2-0.6, 0.3-0.5, 0.2, 0.3, 0.4, 0.5, or 0.6 wt % dissolved sugars. Such dissolved sugars can include sucrose, fructose, leucrose, and/or soluble gluco-oligosaccharides, for example. Isolation herein can optionally further comprise drying graft copolymer, and/or preparing a dispersion of graft copolymer.

An isolated graft copolymer herein as provided in a dry form, can comprise no more than 2.0, 1.5, 1.0, 0.5, 0.25, 0.10, 0.05, or 0.01 wt % water, for example. In some aspects, a graft copolymer is provided in an amount of at least 1 gram (e.g., at least about 2.5, 5, 10, 25, 50, 100, 250, 500, 750, 1000, 2500, 5000, 7500, 10000, 25000, 50000, or 100000 g); such an amount can be a dry amount, for example.

In some aspects, a graft copolymer that has been isolated (optionally characterized as “purified”) can be present in a composition at a wt % (dry weight basis) of at least about 50%, 60%, 70%, 80%, 85%, 90%, 91%, 92%, 93%, 94%, 95%, 96%, 97%, 98%, 98.5%, 99%, 99.5%, 99.8%, or 99.9%. Such isolated graft copolymer can be used as an ingredient/component in a product/application, for example.

Certain embodiments of the present disclosure concern a method of providing an aqueous composition that comprises a graft copolymer. Such a method comprises (a) providing a graft copolymer as presently disclosed, and (b) dispersing or dissolving the graft copolymer into an aqueous liquid, thereby producing an aqueous composition that comprises a graft copolymer. This method can optionally be characterized as a dispersion method, in aspects in which the graft copolymer provided in step (a) is insoluble in aqueous conditions as presently disclosed. Alternatively, the method can optionally be characterized as a dissolution method, in aspects in which the graft copolymer provided in step (a) is soluble in aqueous conditions as presently disclosed.

A graft copolymer provided for use in a dispersion or dissolution method herein can be dry or wet. A dry form of graft copolymer can comprise no more than 2.0, 1.5, 1.0, 0.5, 0.25, 0.10, 0.05, or 0.01 wt % water, for example. A wet form of a graft copolymer can be a “cake” in some aspects. A cake herein refers to a preparation of graft copolymer in condensed, compacted, packed, squeezed, and/or compressed form that comprises at least (i) about 50%-90% by weight aqueous fluid (e.g., water or water-based solution), and (ii) about 10%-50% by weight graft copolymer. A cake can optionally be referred to as a “filter cake” or “wet cake”.

Any suitable method can be employed to perform step (b) of dispersing a graft copolymer. In some aspects, such dispersal can be performed by applying high shear and/or other forms of mixing/agitation. High shear can be of about, or at least about, 8, 9, 10, 11, or 12 kJ/kg in specific energy, and/or can comprise mixing at about, or up to about, 3000, 4000, 6000, 8000, 10000, 12000, 14000, or 15000 rpm, for example. High shear and/or mixing/agitation can be applied for about 1, 2, 3, 4, 5, 6, 8, or 10 minutes, or 2-4 minutes, for example. Suitable means for shearing/mixing/agitating include, for example, a disperser, sonicator (e.g., ultrasonicator) (e.g., 40-60 W, ˜50 W) homomixer, homogenizer (e.g., rotary or piston, rotar-stator), microfluidizer, planetary mixer, colloid mill, jet mill, vortex, and/or any methodology as described in the below Examples and/or in International Patent Appl. Publ. No. WO2016/030234, U.S. Pat. Nos. 5,767,176, 6,139,875, and/or 8,722,092, and/or U.S. Patent Appl. Publ. Nos. 2017/0055540 and/or 2018/0021238, which are all incorporated herein by reference. In some aspects, high shear mixing (such as applied by any of the foregoing means) is not used to disperse a graft copolymer to achieve elevated viscosity; gentle mixing/agitation such at a low rpm/frequency (e.g., less than about 100, 50, or 30 rpm) is used to disperse the graft copolymer in such aspects. A dispersion produced herein can optionally be a colloidal dispersion.

An aqueous composition produced by a dispersion or dissolution method herein can comprise about, at least about, or less than about, 0.1, 0.25, 0.4, 0.5, 0.75, 1.0, 1.25, 1.5, 1.75, 2.0, 2.25, 2.5, 3.0, 4.0, 5.0, 6.0, 7.0, 8.0, 9.0, or 10.0 w/v % graft copolymer, for example, or a range between any two of these values. Merely as examples, an aqueous composition can comprise about 0.1-2.5, 0.1-2.25, 0.1-2.0, 0.1-1.75, 0.1-1.5, 0.1-1.25, 0.1-1.0, 0.1-0.75, 0.1-0.5, 0.25-2.5, 0.25-2.25, 0.25-2.0, 0.25-1.75, 0.25-1.5, 0.25-1.25, 0.25-1.0, 0.25-0.75, 0.25-0.5, 0.5-2.5, 0.5-2.25, 0.5-2.0, 0.5-1.75, 0.5-1.5, 0.5-1.25, 0.5-1.0, or 0.5-0.75 w/v % graft copolymer.

In some aspects, the viscosity of the aqueous composition produced in step (b) of a dispersion or dissolution method is at least about 10%, 50%, 75%, 100%, 500%, 1000%, 10000%, or 100000%, or 1000000% (or any integer between 10% and 100000%) higher than the viscosity of the aqueous liquid as it existed before step (b) of dispersing or dissolving. Very large percent increases in viscosity can be obtained with the disclosed method when the aqueous liquid has little to no viscosity before step (b). The viscosity of an aqueous composition comprising a graft copolymer can be about, or at least about, 100, 200, 300, 400, 500, 600, 700, 1000, 2000, 3000, 4000, 5000, 6000, 7000, 8000, 9000, 10000, 25000, 50000, 75000, or 100000 centipoise (cP), for example. Viscosity can be as measured with an aqueous composition at any temperature between about 3° C. to about 80° C., for example (e.g., 4-30° C., 15-30° C., 15-25° C.). Viscosity typically is as measured at atmospheric pressure (about 760 torr) or a pressure that is within ±10% thereof. Viscosity can be measured using a viscometer or rheometer, for example, and can optionally be as measured at a shear rate (rotational shear rate) of about 0.1, 0.5, 1.0, 5, 10, 50, 100, 500, 1000, 0.1-500, 0.1-100, 1.0-500, or 1.0-100 s⁻¹ (1/s), for example. Viscosity can optionally be measured following the procedure outlined in the below Examples. It is notable that a graft copolymer herein typically has enhanced viscosity (at any given shear rate) compared to alpha-1,3-glucan (each polymer provided in the same amount and having been produced under the same or similar glucosyltransferase reaction conditions, except for the presence of an alpha-1,3-glucan ether or ester primer in the graft copolymer-producing reaction). Such viscosity enhancement can be about, or at least about, a 2-fold, 4-fold, 6-fold, 8-fold, 10-fold, or 12-fold increase in viscosity (at any given shear rate) for a graft copolymer as compared to alpha-1,3-glucan. It is notable that dry forms (e.g., dried at least once following synthesis) and “never-dried” wet forms (the polymer has never been dried following its synthesis) of graft copolymer herein are typically both able to increase viscosity to the same or similar (e.g., within ±10%) extent, whereas alpha-1,3-glucan produced in a reaction without a glucan ether or ester primer typically does not exhibit this beneficial feature; alpha-1,3-glucan that has been dried at least once typically is not capable of increasing viscosity to the same or similar (e.g., within ±10%) extent as never-dried wet forms of alpha-1,3-glucan.

A graft copolymer herein can be present in a composition, such as an aqueous composition (e.g., dispersion such as colloidal dispersion) or dry composition, at about, at least about, or less than about, 0.01, 0.05, 0.1, 0.2, 0.25, 0.3, 0.4, 0.5, 0.6, 0.7, 0.75, 0.8, 0.9, 1.0, 1.2, 1.25, 1.4, 1.5, 1.6, 1.75, 1.8, 2.0, 2.25, 2.5, 3.0, 3.5, 4.0, 4.5, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 wt % or w/v %, for example, or a range between any two of these values. Merely as examples, an aqueous composition can comprise about 0.1-2.5, 0.1-2.25, 0.1-2.0, 0.1-1.75, 0.1-1.5, 0.1-1.25, 0.1-1.0, 0.1-0.75, 0.1-0.5, 0.25-2.5, 0.25-2.25, 0.25-2.0, 0.25-1.75, 0.25-1.5, 0.25-1.25, 0.25-1.0, 0.25-0.75, 0.25-0.5, 0.5-2.5, 0.5-2.25, 0.5-2.0, 0.5-1.75, 0.5-1.5, 0.5-1.25, 0.5-1.0, or 0.5-0.75 w/v % or wt % graft copolymer. The liquid component of an aqueous composition can be an aqueous fluid such as water or aqueous solution, for instance. The solvent of an aqueous solution typically is water, or can comprise about, or at least about, 10, 20, 30, 40, 50, 60, 70, 80, 90, 95, 98, or 99 wt % water, for example. A graft copolymer as comprised in a dispersion herein typically is aqueous insoluble. A composition herein can optionally further comprise alpha-1,3-glucan homopolymer.

An aqueous solution in some aspects has no (detectable) dissolved sugars, or about 0.1-1.5, 0.1-1.25, 0.1-1.0, 0.1-0.75, 0.1-0.5, 0.2-0.6, 0.3-0.5, 0.2, 0.3, 0.4, 0.5, or 0.6 wt % dissolved sugars. Such dissolved sugars can include sucrose, fructose, leucrose, and/or soluble gluco-oligosaccharides, for example. An aqueous solution in some aspects can have one or more salts/buffers (e.g., Na⁺, Cl⁻, NaCl, phosphate, tris, citrate) (e.g., ≤0.1, 0.5, 1.0, 2.0, or 3.0 wt %) and/or a pH of about 4.0, 4.5, 5.0, 5.5, 6.0, 6.5, 7.0, 7.5, 8.0, 8.5, 9.0, 4.0-9.0, 4.0-8.5, 4.0-8.0, 5.0-9.0, 5.0-8.5, 5.0-8.0, 6.0-9.0, 6.0-8.5, or 6.0-8.0, for example.

A composition comprising a graft copolymer (e.g., a dispersion if the polymer is insoluble, or a solution if the polymer is soluble) can have a viscosity of about, or at least about, 100, 200, 300, 400, 500, 600, 700, 1000, 2000, 3000, 4000, 5000, 6000, 7000, 8000, 9000, 10000, 25000, 50000, 75000, or 100000 centipoise (cP), for example. Viscosity can be as measured with an aqueous composition at any temperature between about 3° C. to about 80° C., for example (e.g., 4-30° C., 15-30° C., 15-25° C.). Viscosity typically is as measured at atmospheric pressure (about 760 torr) or a pressure that is within ±10% thereof. Viscosity can be measured using a viscometer or rheometer, for example, and can optionally be as measured at a shear rate (rotational shear rate) of about 0.1, 0.5, 1.0, 5, 10, 50, 100, 500, 1000, 0.1-500, 0.1-100, 1.0-500, or 1.0-100 s⁻¹ (1/s), for example. Viscosity can optionally be measured following the procedure outlined in the below Examples.

The zeta potential of a graft copolymer in an aqueous composition (e.g., dispersion) in some aspects can be over ±15 mV, ±17.5 mV, ±20 mV, ±22.5 mV, ±25 mV, ±27.5 mV, ±30 mV, ±32.5 mV, ±35 mV, ±37.5 mV, or ±40 mV. Simply for illustration purposes, it should be understood that a zeta potential over ±15 mV, for example, excludes zeta potentials ranging from −15 mV to +15 mV. In some aspects, the zeta potential is ±20 to ±40 mV, ±20 to ±30 mV, ±25 to ±40 mV, or ±25 to ±30 mV. In general, it is contemplated that the zeta potential of a graft copolymer comprising an anionic alpha-1,3-glucan derivative backbone has a greater negative value than −15 mV, and that the zeta potential of a graft copolymer comprising a cationic alpha-1,3-glucan derivative backbone has a greater positive value than +15 mV. The foregoing zeta potential values can in some aspects can be associated with aqueous compositions having a pH of about 6-8 or 5-9. It is notable that a graft copolymer herein typically has a greater positive or negative zeta potential value compared to alpha-1,3-glucan (each polymer provided in the same manner and having been produced under the same or similar glucosyltransferase reaction conditions, except for the presence of an alpha-1,3-glucan ether or ester primer in the graft copolymer-producing reaction). Such enhancement can be by an absolute difference in zeta potential values of at least 10, 15, 20, 25, 30, or 40, for example. Zeta potential herein can be measured as described in the below Examples, and/or as disclosed, for example, in U.S. Pat. Nos. 6,109,098 and/or 4,602,989, and/or Int. Patent Appl. Publ. Nos. WO2014/097402 and/or EP0869357, which are incorporated herein by reference.

In some aspects, the particle size of a graft copolymer in an aqueous composition (e.g., dispersion) is less than 2.0, 1.8, 1.6, 1.5, 1.4, 1.3, 1.2, 1.1, 1.0, 0.9, 0.8, 0.7, 0.6, 0.5, 0.4, 0.3, or 0.2 micron. Particle size can be 0.15-0.5, 0.15-0.4, 0.15-0.3, 0.2-0.5, 0.2-0.4, or 0.2-0.3 in some aspects. Particle size herein can optionally be in terms of a “D50” (diameter-50) value of a sample of graft copolymer particles. A D50 measurement for a sample of graph copolymer particles herein is the particle diameter at which (i) 50% of the sample's mass is comprised of particles with a diameter less than this measurement, and (ii) 50% of the sample's mass is comprised of particles with a diameter greater than this measurement. It is notable that graft copolymer particles herein typically are of a reduced size compared to the particle size of alpha-1,3-glucan (each polymer provided in the same manner and having been produced under the same or similar glucosyltransferase reaction conditions, except for the presence of an alpha-1,3-glucan ether or ester primer in the graft copolymer-producing reaction). Such size reduction can be by about at least 80%, 85%, 90%, 95%, 96%, 97%, 98%, 99%, 99.5%, 99.8%, or 99.9%, for example. Without intending to be held to any particular theory, it is contemplated that retention of small particle size by graft copolymer particles herein (as opposed to agglomerating/flocculating to form larger particles) is due at least in part to the enhanced zeta potential of the particles. Graft copolymer particles can be prepared using a mixing/agitation means as disclosed herein such as sonication, for example, and particle size can be measured as described in the below Examples, and/or as disclosed, for example, in U.S. Pat. Nos. 6,109,098, 9,869,625, and/or 5,831,150, which are incorporated herein by reference.

A composition comprising a graft copolymer herein can, in some aspects, be non-aqueous (e.g., a dry composition). Examples of such embodiments include powders, granules, microcapsules, flakes, or any other form of particulate matter. Other examples include larger compositions such as pellets, bars, kernels, beads, tablets, sticks, or other agglomerates. A non-aqueous or dry composition typically has less than 2.0, 1.5, 1.0, 0.5, 0.25, 0.10, 0.05, or 0.01 wt % water comprised therein.

A composition comprising a graft copolymer herein can, in some aspects, comprise one or more salts such as a sodium salt (e.g., NaCl, Na₂SO₄). Other non-limiting examples of salts include those having (i) an aluminum, ammonium, barium, calcium, chromium (II or III), copper (I or II), iron (II or III), hydrogen, lead (II), lithium, magnesium, manganese (II or III), mercury (I or II), potassium, silver, sodium strontium, tin (II or IV), or zinc cation, and (ii) an acetate, borate, bromate, bromide, carbonate, chlorate, chloride, chlorite, chromate, cyanamide, cyanide, dichromate, dihydrogen phosphate, ferricyanide, ferrocyanide, fluoride, hydrogen carbonate, hydrogen phosphate, hydrogen sulfate, hydrogen sulfide, hydrogen sulfite, hydride, hydroxide, hypochlorite, iodate, iodide, nitrate, nitride, nitrite, oxalate, oxide, perchlorate, permanganate, peroxide, phosphate, phosphide, phosphite, silicate, stannate, stannite, sulfate, sulfide, sulfite, tartrate, or thiocyanate anion. Thus, any salt having a cation from (i) above and an anion from (ii) above can be in a composition, for example. A salt can be present in an aqueous composition herein at a wt % of about, or at least about, 0.01, 0.025, 0.05, 0.075, 0.1, 0.25, 0.5, 0.75, 1.0, 1.25, 1.5, 1.75, 2.0, 2.5, 3.0, 3.5, 0.01-3.5, 0.5-3.5, 0.5-2.5, or 0.5-1.5 wt % (such wt % values typically refer to the total concentration of one or more salts), for example.

A composition herein comprising a graft copolymer can optionally contain one or more active enzymes. Examples of suitable enzymes include proteases, cellulases, hemicellulases, peroxidases, lipolytic enzymes (e.g., metallolipolytic enzymes), xylanases, lipases, phospholipases, esterases (e.g., arylesterase, polyesterase), perhydrolases, cutinases, pectinases, pectate lyases, mannanases, keratinases, reductases, oxidases (e.g., choline oxidase), phenoloxidases, lipoxygenases, ligninases, pullulanases, tannases, pentosanases, malanases, beta-glucanases, arabinosidases, hyaluronidases, chondroitinases, laccases, metalloproteinases, amadoriases, glucoamylases, arabinofuranosidases, phytases, isomerases, transferases and amylases. If an enzyme(s) is included, it may be comprised in a composition herein at about 0.0001-0.1 wt % (e.g., 0.01-0.03 wt %) active enzyme (e.g., calculated as pure enzyme protein), for example. In fabric care applications, cellulase can be present in an aqueous composition in which a fabric is treated (e.g., wash liquor) at a concentration that is minimally about 0.01-0.1 ppm total cellulase protein, or about 0.1-10 ppb total cellulase protein (e.g., less than 1 ppm), to maximally about 100, 200, 500, 1000, 2000, 3000, 4000, or 5000 ppm total cellulase protein, for example.

A composition comprising a graft copolymer herein, such as an aqueous composition or a non-aqueous composition (above), can be in the form of a household care product, personal care product, industrial product, pharmaceutical product, or food product, for example, such as described in any of U.S. Patent Appl. Publ. Nos. 2018/0022834, 2018/0237816, 20180079832, 2016/0311935, 2016/0304629, 2015/0232785, 2015/0368594, 2015/0368595, 2019/0202942, and/or 2016/0122445, and/or Int. Patent Appl. Publ. Nos. WO2016/160737, WO2016/160738, WO2016/133734, and/or WO2016/160740, which are all incorporated herein by reference. In some aspects, a composition comprising a graft copolymer can comprise at least one component/ingredient of a household care product, personal care product, industrial product, pharmaceutical product, or food product as disclosed in any of the foregoing publications and/or as presently disclosed.

Graft copolymers disclosed herein are believed to be useful for providing one or more of the following physical properties to a personal care product, pharmaceutical product, household product, industrial product, or food product: thickening, freeze/thaw stability, lubricity, moisture retention and release, texture, consistency, shape retention, emulsification, binding, suspension, dispersion, gelation, reduced mineral hardness, for example. Examples of a concentration or amount of a graft copolymer in a product can be any of the weight percentages provided herein, for example.

Personal care products herein are not particularly limited and include, for example, skin care compositions, cosmetic compositions, antifungal compositions, and antibacterial compositions. Personal care products herein may be in the form of, for example, lotions, creams, pastes, balms, ointments, pomades, gels, liquids, combinations of these and the like. The personal care products disclosed herein can include at least one active ingredient, if desired. An active ingredient is generally recognized as an ingredient that causes an intended pharmacological effect.

In certain embodiments, a skin care product can be applied to skin for addressing skin damage related to a lack of moisture. A skin care product may also be used to address the visual appearance of skin (e.g., reduce the appearance of flaky, cracked, and/or red skin) and/or the tactile feel of the skin (e.g., reduce roughness and/or dryness of the skin while improved the softness and subtleness of the skin). A skin care product typically may include at least one active ingredient for the treatment or prevention of skin ailments, providing a cosmetic effect, or for providing a moisturizing benefit to skin, such as zinc oxide, petrolatum, white petrolatum, mineral oil, cod liver oil, lanolin, dimethicone, hard fat, vitamin A, allantoin, calamine, kaolin, glycerin, or colloidal oatmeal, and combinations of these. A skin care product may include one or more natural moisturizing factors such as ceramides, hyaluronic acid, glycerin, squalane, amino acids, cholesterol, fatty acids, triglycerides, phospholipids, glycosphingolipids, urea, linoleic acid, glycosaminoglycans, mucopolysaccharide, sodium lactate, or sodium pyrrolidone carboxylate, for example. Other ingredients that may be included in a skin care product include, without limitation, glycerides, apricot kernel oil, canola oil, squalane, squalene, coconut oil, corn oil, jojoba oil, jojoba wax, lecithin, olive oil, safflower oil, sesame oil, shea butter, soybean oil, sweet almond oil, sunflower oil, tea tree oil, shea butter, palm oil, cholesterol, cholesterol esters, wax esters, fatty acids, and orange oil.

A personal care product herein can also be in the form of makeup, lipstick, mascara, rouge, foundation, blush, eyeliner, lip liner, lip gloss, other cosmetics, sunscreen, sun block, nail polish, nail conditioner, bath gel, shower gel, body wash, face wash, lip balm, skin conditioner, cold cream, moisturizer, body spray, soap, body scrub, exfoliant, astringent, scruffing lotion, depilatory, permanent waving solution, antidandruff formulation, antiperspirant composition, deodorant, shaving product, pre-shaving product, after-shaving product, cleanser, skin gel, rinse, dentifrice composition, toothpaste, or mouthwash, for example. An example of a personal care product (e.g., a cleanser, soap, scrub, cosmetic) comprises a carrier or exfoliation agent (e.g., jojoba beads [jojoba ester beads]) (e.g., about 1-10, 3-7, 4-6, or 5 wt %); such an agent may optionally be dispersed within the product.

A personal care product in some aspects can be a hair care product. Examples of hair care products herein include shampoo, hair conditioner (leave-in or rinse-out), cream rinse, hair dye, hair coloring product, hair shine product, hair serum, hair anti-frizz product, hair split-end repair product, mousse, hair spray, and styling gel. A hair care product can be in the form of a liquid, paste, gel, solid, or powder in some embodiments. A hair care product as presently disclosed typically comprises one or more of the following ingredients, which are generally used to formulate hair care products: anionic surfactants such as polyoxyethylenelauryl ether sodium sulfate; cationic surfactants such as stearyltrimethylammonium chloride and/or distearyltrimethylammonium chloride; nonionic surfactants such as glyceryl monostearate, sorbitan monopalmitate and/or polyoxyethylenecetyl ether; wetting agents such as propylene glycol, 1,3-butylene glycol, glycerin, sorbitol, pyroglutamic acid salts, amino acids and/or trimethylglycine; hydrocarbons such as liquid paraffins, petrolatum, solid paraffins, squalane and/or olefin oligomers; higher alcohols such as stearyl alcohol and/or cetyl alcohol; superfatting agents; antidandruff agents; disinfectants; anti-inflammatory agents; crude drugs; water-soluble polymers such as methyl cellulose, hydroxycellulose and/or partially deacetylated chitin; antiseptics such as paraben; ultra-violet light absorbers; pearling agents; pH adjustors; perfumes; and pigments.

A pharmaceutical product herein can be in the form of an emulsion, liquid, elixir, gel, suspension, solution, cream, or ointment, for example. Also, a pharmaceutical product herein can be in the form of any of the personal care products disclosed herein, such as an antibacterial or antifungal composition. A pharmaceutical product can further comprise one or more pharmaceutically acceptable carriers, diluents, and/or pharmaceutically acceptable salts. A graft copolymer disclosed herein can also be used in capsules, encapsulants, tablet coatings, and as an excipients for medicaments and drugs.

A household and/or industrial product herein can be in the form of drywall tape-joint compounds; mortars; grouts; cement plasters; spray plasters; cement stucco; adhesives; pastes; wall/ceiling texturizers; binders and processing aids for tape casting, extrusion forming, injection molding and ceramics; spray adherents and suspending/dispersing aids for pesticides, herbicides, and fertilizers; fabric care products such as fabric softeners and laundry detergents; hard surface cleaners; air fresheners; polymer emulsions; gels such as water-based gels; surfactant solutions; paints such as water-based paints; protective coatings; adhesives; sealants and caulks; inks such as water-based ink; metal-working fluids; films or coatings; or emulsion-based metal cleaning fluids used in electroplating, phosphatizing, galvanizing and/or general metal cleaning operations, for example.

A graft copolymer disclosed herein can be comprised in a personal care product, pharmaceutical product, household product, or industrial product in an amount that provides a desired degree of thickening and/or dispersion, for example. Examples of a concentration or amount of a graft copolymer in a product can be any of the weight percentages provided above, for example.

Compositions disclosed herein can be in the form of a fabric care composition. A fabric care composition herein can be used for hand wash, machine wash and/or other purposes such as soaking and/or pretreatment of fabrics, for example. A fabric care composition may take the form of, for example, a laundry detergent; fabric conditioner; any wash-, rinse-, or dryer-added product; unit dose or spray. Fabric care compositions in a liquid form may be in the form of an aqueous composition as disclosed herein. In other aspects, a fabric care composition can be in a dry form such as a granular detergent or dryer-added fabric softener sheet. Other non-limiting examples of fabric care compositions herein include: granular or powder-form all-purpose or heavy-duty washing agents; liquid, gel or paste-form all-purpose or heavy-duty washing agents; liquid or dry fine-fabric (e.g. delicates) detergents; cleaning auxiliaries such as bleach additives, “stain-stick”, or pre-treatments; substrate-laden products such as dry and wetted wipes, pads, or sponges; sprays and mists.

A detergent composition herein may be in any useful form, e.g., as powders, granules, pastes, bars, unit dose, or liquid. A liquid detergent may be aqueous, typically containing up to about 70 wt % of water and 0 wt % to about 30 wt % of organic solvent. It may also be in the form of a compact gel type containing only about 30 wt % water.

A detergent composition herein typically comprises one or more surfactants, wherein the surfactant is selected from nonionic surfactants, anionic surfactants, cationic surfactants, ampholytic surfactants, zwitterionic surfactants, semi-polar nonionic surfactants and mixtures thereof. In some embodiments, the surfactant is present at a level of from about 0.1% to about 60%, while in alternative embodiments the level is from about 1% to about 50%, while in still further embodiments the level is from about 5% to about 40%, by weight of the detergent composition. A detergent will usually contain 0 wt % to about 50 wt % of an anionic surfactant such as linear alkylbenzenesulfonate (LAS), alpha-olefinsulfonate (AOS), alkyl sulfate (fatty alcohol sulfate) (AS), alcohol ethoxysulfate (AEOS or AES), secondary alkanesulfonates (SAS), alpha-sulfo fatty acid methyl esters, alkyl- or alkenylsuccinic acid, or soap. In addition, a detergent composition may optionally contain 0 wt % to about 40 wt % of a nonionic surfactant such as alcohol ethoxylate (AEO or AE), carboxylated alcohol ethoxylates, nonylphenol ethoxylate, alkylpolyglycoside, alkyldimethylamineoxide, ethoxylated fatty acid monoethanolamide, fatty acid monoethanolamide, or polyhydroxy alkyl fatty acid amide (as described for example in WO92/06154, which is incorporated herein by reference).

A detergent composition herein typically comprises one or more detergent builders or builder systems. In some aspects, oxidized poly alpha-1,3-glucan can be included as a co-builder, in which it is used together with one or more additional builders such as any disclosed herein. Oxidized poly alpha-1,3-glucan compounds for use herein are disclosed in U.S. Patent Appl. Publ. No. 2015/0259439. In some embodiments incorporating at least one builder, the cleaning compositions comprise at least about 1%, from about 3% to about 60%, or even from about 5% to about 40%, builder by weight of the composition. Builders (in addition to oxidized poly alpha-1,3-glucan) include, but are not limited to, alkali metal, ammonium and alkanolammonium salts of polyphosphates, alkali metal silicates, alkaline earth and alkali metal carbonates, aluminosilicates, polycarboxylate compounds, ether hydroxypolycarboxylates, copolymers of maleic anhydride with ethylene or vinyl methyl ether, 1,3,5-trihydroxy benzene-2,4,6-trisulphonic acid, and carboxymethyloxysuccinic acid, various alkali metal, ammonium and substituted ammonium salts of polyacetic acids such as ethylenediamine tetraacetic acid and nitrilotriacetic acid, as well as polycarboxylates such as mellitic acid, succinic acid, citric acid, oxydisuccinic acid, polymaleic acid, benzene 1,3,5-tricarboxylic acid, carboxymethyloxysuccinic acid, and soluble salts thereof. Indeed, it is contemplated that any suitable builder will find use in various embodiments of the present disclosure. Additional examples of a detergent builder or complexing agent include zeolite, diphosphate, triphosphate, phosphonate, citrate, nitrilotriacetic acid (NTA), ethylenediaminetetraacetic acid (EDTA), diethylenetriaminepentaacetic acid (DTMPA), alkyl-or alkenylsuccinic acid, soluble silicates or layered silicates (e.g., SKS-6 from Hoechst).

In some embodiments, builders form water-soluble hardness ion complexes (e.g., sequestering builders), such as citrates and polyphosphates (e.g., sodium tripolyphosphate and sodium tripolyphospate hexahydrate, potassium tripolyphosphate, and mixed sodium and potassium tripolyphosphate, etc.). It is contemplated that any suitable builder will find use in the present disclosure, including those known in the art (See, e.g., EP2100949).

In some embodiments, suitable builders can include phosphate builders and non-phosphate builders. In some embodiments, a builder is a phosphate builder. In some embodiments, a builder is a non-phosphate builder. A builder can be used in a level of from 0.1% to 80%, or from 5% to 60%, or from 10% to 50%, by weight of the composition. In some embodiments, the product comprises a mixture of phosphate and non-phosphate builders. Suitable phosphate builders include mono-phosphates, di-phosphates, tri-polyphosphates or oligomeric-polyphosphates, including the alkali metal salts of these compounds, including the sodium salts. In some embodiments, a builder can be sodium tripolyphosphate (STPP). Additionally, the composition can comprise carbonate and/or citrate, preferably citrate that helps to achieve a neutral pH composition. Other suitable non-phosphate builders include homopolymers and copolymers of polycarboxylic acids and their partially or completely neutralized salts, monomeric polycarboxylic acids and hydroxycarboxylic acids and their salts. In some embodiments, salts of the above mentioned compounds include ammonium and/or alkali metal salts, i.e., lithium, sodium, and potassium salts, including sodium salts. Suitable polycarboxylic acids include acyclic, alicyclic, hetero-cyclic and aromatic carboxylic acids, wherein in some embodiments, they can contain at least two carboxyl groups which are in each case separated from one another by, in some instances, no more than two carbon atoms.

A detergent composition herein can comprise at least one chelating agent. Suitable chelating agents include, but are not limited to copper, iron and/or manganese chelating agents and mixtures thereof. In embodiments in which at least one chelating agent is used, the composition comprises from about 0.1% to about 15%, or even from about 3.0% to about 10%, chelating agent by weight of the composition.

A detergent composition herein can comprise at least one deposition aid. Suitable deposition aids include, but are not limited to, polyethylene glycol, polypropylene glycol, polycarboxylate, soil release polymers such as polytelephthalic acid, clays such as kaolinite, montmorillonite, atapulgite, illite, bentonite, halloysite, and mixtures thereof.

A detergent composition herein can comprise one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. Additional dye transfer inhibiting agents include manganese phthalocyanine, peroxidases, polyvinylpyrrolidone polymers, polyamine N-oxide polymers, copolymers of N-vinylpyrrolidone and N-vinylimidazole, polyvinyloxazolidones and polyvinylimidazoles and/or mixtures thereof; chelating agents examples of which include ethylene-diamine-tetraacetic acid (EDTA); diethylene triamine penta methylene phosphonic acid (DTPMP); hydroxy-ethane diphosphonic acid (HEDP); ethylenediamine N,N′-disuccinic acid (EDDS); methyl glycine diacetic acid (MGDA); diethylene triamine penta acetic acid (DTPA); propylene diamine tetraacetic acid (PDT A); 2-hydroxypyridine-N-oxide (HPNO); or methyl glycine diacetic acid (MGDA); glutamic acid N,N-diacetic acid (N,N-dicarboxymethyl glutamic acid tetrasodium salt (GLDA); nitrilotriacetic acid (NTA); 4,5-dihydroxy-m-benzenedisulfonic acid; citric acid and any salts thereof; N-hydroxyethyl ethylenediaminetri-acetic acid (HEDTA), triethylenetetraaminehexaacetic acid (TTHA), N-hydroxyethyliminodiacetic acid (HEIDA), dihydroxyethylglycine (DHEG), ethylenediaminetetrapropionic acid (EDTP) and derivatives thereof, which can be used alone or in combination with any of the above. In embodiments in which at least one dye transfer inhibiting agent is used, a composition herein may comprise from about 0.0001% to about 10%, from about 0.01% to about 5%, or even from about 0.1% to about 3%, by weight of the composition.

A detergent composition herein can comprise silicates. In some of these embodiments, sodium silicates (e.g., sodium disilicate, sodium metasilicate, and/or crystalline phyllosilicates) find use. In some embodiments, silicates are present at a level of from about 1% to about 20% by weight of the composition. In some embodiments, silicates are present at a level of from about 5% to about 15% by weight of the composition.

A detergent composition herein can comprise dispersants. Suitable water-soluble organic materials include, but are not limited to the homo-or co-polymeric acids or their salts, in which the polycarboxylic acid comprises at least two carboxyl radicals separated from each other by not more than two carbon atoms.

A detergent composition herein may additionally comprise one or more enzymes. Examples of enzymes include proteases, cellulases, hemicellulases, peroxidases, lipolytic enzymes (e.g., metallolipolytic enzymes), xylanases, lipases, phospholipases, esterases (e.g., arylesterase, polyesterase), perhydrolases, cutinases, pectinases, pectate lyases, mannanases, keratinases, reductases, oxidases (e.g., choline oxidase, phenoloxidase), phenoloxidases, lipoxygenases, ligninases, pullulanases, tannases, pentosanases, malanases, beta-glucanases, arabinosidases, hyaluronidases, chondroitinases, laccases, metalloproteinases, amadoriases, glucoamylases, alpha-amylases, beta-amylases, galactosidases, galactanases, catalases, carageenases, hyaluronidases, keratinases, lactases, ligninases, peroxidases, phosphatases, polygalacturonases, pullulanases, rhamnogalactouronases, tannases, transglutaminases, xyloglucanases, xylosidases, metalloproteases, arabinofuranosidases, phytases, isomerases, transferases and/or amylases in any combination.

In some embodiments, a detergent composition can comprise one or more enzymes (e.g., any disclosed herein), each at a level from about 0.00001% to about 10% by weight of the composition and the balance of cleaning adjunct materials by weight of composition. In some other embodiments, a detergent composition can also comprise each enzyme at a level of about 0.0001% to about 10%, about 0.001% to about 5%, about 0.001% to about 2%, or about 0.005% to about 0.5%, by weight of the composition.

Enzymes that may be comprised in a detergent composition herein may be stabilized using conventional stabilizing agents, e.g., a polyol such as propylene glycol or glycerol; a sugar or sugar alcohol; lactic acid; boric acid or a boric acid derivative (e.g., an aromatic borate ester).

A detergent composition in certain embodiments may comprise one or more other types of polymers in addition to a graft copolymer as disclosed herein. Examples of other types of polymers useful herein include carboxymethyl cellulose (CMC), dextran, poly(vinylpyrrolidone) (PVP), polyethylene glycol (PEG), poly(vinyl alcohol) (PVA), polycarboxylates such as polyacrylates, maleic/acrylic acid copolymers and lauryl methacrylate/acrylic acid copolymers.

A detergent composition herein may contain a bleaching system. For example, a bleaching system can comprise an H₂O₂ source such as perborate or percarbonate, which may be combined with a peracid-forming bleach activator such as tetraacetylethylenediamine (TAED) or nonanoyloxybenzenesulfonate (NOBS). Alternatively, a bleaching system may comprise peroxyacids (e.g., amide, imide, or sulfone type peroxyacids). Alternatively still, a bleaching system can be an enzymatic bleaching system comprising perhydrolase, for example, such as the system described in WO2005/056783.

A detergent composition herein may also contain conventional detergent ingredients such as fabric conditioners, clays, foam boosters, suds suppressors, anti-corrosion agents, soil-suspending agents, anti-soil redeposition agents, dyes, bactericides, tarnish inhibiters, optical brighteners, or perfumes. The pH of a detergent composition herein (measured in aqueous solution at use concentration) is usually neutral or alkaline (e.g., pH of about 7.0 to about 11.0).

It is believed that a graft copolymer herein can be included as an anti-redeposition agent and/or clay soil removal agent in a detergent composition such as a fabric care composition, if desired (such agents can optionally be characterized as whiteness maintenance agents in certain aspects). Examples of other suitable anti-redeposition and/or clay soil removal agents herein include polyethoxy zwitterionic surfactants, water-soluble copolymers of acrylic or methacrylic acid with acrylic or methacrylic acid-ethylene oxide condensates (e.g., U.S. Pat. No. 3,719,647), cellulose derivatives such as carboxymethylcellulose and hydroxypropylcellulose (e.g., U.S. Pat. Nos. 3,597,416 and 3,523,088), and mixtures comprising nonionic alkyl polyethoxy surfactant, polyethoxy alkyl quaternary cationic surfactant and fatty amide surfactant (e.g., U.S. Pat. No. 4,228,044). Non-limiting examples of other suitable anti-redeposition and clay soil removal agents are disclosed in U.S. Pat. Nos. 4,597,898 and 4,891,160, and Int. Patent Appl. Publ. No. WO95/32272, all of which are incorporated herein by reference.

Particular forms of detergent compositions that can be adapted for purposes disclosed herein are disclosed in, for example, US20090209445A1, US20100081598A1, U.S. Pat. No. 7,001,878B2, EP1504994B1, WO2001085888A2, WO2003089562A1, WO2009098659A1, WO2009098660A1, WO2009112992A1, WO2009124160A1, WO2009152031A1, WO2010059483A1, WO2010088112A1, WO2010090915A1, WO2010135238A1, WO2011094687A1, WO2011094690A1, WO2011127102A1, WO2011163428A1, WO2008000567A1, WO2006045391A1, WO2006007911A1, WO2012027404A1, EP1740690B1, WO2012059336A1, U.S. Pat. No. 6,730,646B1, WO2008087426A1, WO2010116139A1, and WO2012104613A1, all of which are incorporated herein by reference.

Laundry detergent compositions herein can optionally be heavy duty (all purpose) laundry detergent compositions. Exemplary heavy duty laundry detergent compositions comprise a detersive surfactant (10%-40% wt/wt), including an anionic detersive surfactant (selected from a group of linear or branched or random chain, substituted or unsubstituted alkyl sulphates, alkyl sulphonates, alkyl alkoxylated sulphate, alkyl phosphates, alkyl phosphonates, alkyl carboxylates, and/or mixtures thereof), and optionally non-ionic surfactant (selected from a group of linear or branched or random chain, substituted or unsubstituted alkyl alkoxylated alcohol, e.g., C8-C18 alkyl ethoxylated alcohols and/or C6-C12 alkyl phenol alkoxylates), where the weight ratio of anionic detersive surfactant (with a hydrophilic index (Hlc) of from 6.0 to 9) to non-ionic detersive surfactant is greater than 1:1. Suitable detersive surfactants also include cationic detersive surfactants (selected from a group of alkyl pyridinium compounds, alkyl quaternary ammonium compounds, alkyl quaternary phosphonium compounds, alkyl ternary sulphonium compounds, and/or mixtures thereof); zwitterionic and/or amphoteric detersive surfactants (selected from a group of alkanolamine sulpho-betaines); ampholytic surfactants; semi-polar non-ionic surfactants and mixtures thereof.

A detergent herein such as a heavy duty laundry detergent composition may optionally include, a surfactancy boosting polymer consisting of amphiphilic alkoxylated grease cleaning polymers (selected from a group of alkoxylated polymers having branched hydrophilic and hydrophobic properties, such as alkoxylated polyalkylenimines in the range of 0.05 wt %-10 wt %) and/or random graft polymers (typically comprising of hydrophilic backbone comprising monomers selected from the group consisting of: unsaturated C1-C6 carboxylic acids, ethers, alcohols, aldehydes, ketones, esters, sugar units, alkoxy units, maleic anhydride, saturated polyalcohols such as glycerol, and mixtures thereof; and hydrophobic side chain(s) selected from the group consisting of: C4-C25 alkyl group, polypropylene, polybutylene, vinyl ester of a saturated C1-C6 mono-carboxylic acid, C1-C6 alkyl ester of acrylic or methacrylic acid, and mixtures thereof.

A detergent herein such as a heavy duty laundry detergent composition may optionally include additional polymers such as soil release polymers (include anionically end-capped polyesters, for example SRP1, polymers comprising at least one monomer unit selected from saccharide, dicarboxylic acid, polyol and combinations thereof, in random or block configuration, ethylene terephthalate-based polymers and co-polymers thereof in random or block configuration, for example REPEL-O-TEX SF, SF-2 AND SRP6, TEXCARE SRA100, SRA300, SRN100, SRN170, SRN240, SRN300 AND SRN325, MARLOQUEST SL), anti-redeposition agent(s) herein (0.1 wt % to 10 wt %), include carboxylate polymers, such as polymers comprising at least one monomer selected from acrylic acid, maleic acid (or maleic anhydride), fumaric acid, itaconic acid, aconitic acid, mesaconic acid, citraconic acid, methylenemalonic acid, and any mixture thereof, vinylpyrrolidone homopolymer, and/or polyethylene glycol, molecular weight in the range of from 500 to 100,000 Da); and polymeric carboxylate (such as maleate/acrylate random copolymer or polyacrylate homopolymer).

A detergent herein such as a heavy duty laundry detergent composition may optionally further include saturated or unsaturated fatty acids, preferably saturated or unsaturated C12-C24 fatty acids (0 wt % to 10 wt %); deposition aids (examples for which include polysaccharides, cellulosic polymers, poly diallyl dimethyl ammonium halides (DADMAC), and co-polymers of DAD MAC with vinyl pyrrolidone, acrylamides, imidazoles, imidazolinium halides, and mixtures thereof, in random or block configuration, cationic guar gum, cationic starch, cationic polyacrylamides, and mixtures thereof.

A detergent herein such as a heavy duty laundry detergent composition may optionally further include dye transfer inhibiting agents, examples of which include manganese phthalocyanine, peroxidases, polyvinylpyrrolidone polymers, polyamine N-oxide polymers, copolymers of N-vinylpyrrolidone and N-vinylimidazole, polyvinyloxazolidones and polyvinylimidazoles and/or mixtures thereof; chelating agents, examples of which include ethylene-diamine-tetraacetic acid (EDTA), diethylene triamine penta methylene phosphonic acid (DTPMP), hydroxy-ethane diphosphonic acid (HEDP), ethylenediamine N,N′-disuccinic acid (EDDS), methyl glycine diacetic acid (MGDA), diethylene triamine penta acetic acid (DTPA), propylene diamine tetraacetic acid (PDTA), 2-hydroxypyridine-N-oxide (HPNO), or methyl glycine diacetic acid (MGDA), glutamic acid N,N-diacetic acid (N,N-dicarboxymethyl glutamic acid tetrasodium salt (GLDA), nitrilotriacetic acid (NTA), 4,5-dihydroxy-m-benzenedisulfonic acid, citric acid and any salts thereof, N-hydroxyethylethylenediaminetriacetic acid (HEDTA), triethylenetetraaminehexaacetic acid (TTHA), N-hydroxyethyliminodiacetic acid (HEIDA), dihydroxyethylglycine (DHEG), ethylenediaminetetrapropionic acid (EDTP), and derivatives thereof.

A detergent herein such as a heavy duty laundry detergent composition may optionally include silicone or fatty-acid based suds suppressors; hueing dyes, calcium and magnesium cations, visual signaling ingredients, anti-foam (0.001 wt % to about 4.0 wt %), and/or a structurant/thickener (0.01 wt % to 5 wt %) selected from the group consisting of diglycerides and triglycerides, ethylene glycol distearate, microcrystalline cellulose, microfiber cellulose, biopolymers, xanthan gum, gellan gum, and mixtures thereof). Such structurant/thickener would be, in certain embodiments, in addition to the one or more graft copolymers comprised in the detergent. A structurant can also be referred to as a structural agent.

A detergent herein can be in the form of a heavy duty dry/solid laundry detergent composition, for example. Such a detergent may include: (i) a detersive surfactant, such as any anionic detersive surfactant disclosed herein, any non-ionic detersive surfactant disclosed herein, any cationic detersive surfactant disclosed herein, any zwitterionic and/or amphoteric detersive surfactant disclosed herein, any ampholytic surfactant, any semi-polar non-ionic surfactant, and mixtures thereof; (ii) a builder, such as any phosphate-free builder (e.g., zeolite builders in the range of 0 wt % to less than 10 wt %), any phosphate builder (e.g., sodium tri-polyphosphate in the range of 0 wt % to less than 10 wt %), citric acid, citrate salts and nitrilotriacetic acid, any silicate salt (e.g., sodium or potassium silicate or sodium meta-silicate in the range of 0 wt % to less than 10 wt %); any carbonate salt (e.g., sodium carbonate and/or sodium bicarbonate in the range of 0 wt % to less than 80 wt %), and mixtures thereof; (iii) a bleaching agent, such as any photobleach (e.g., sulfonated zinc phthalocyanines, sulfonated aluminum phthalocyanines, xanthenes dyes, and mixtures thereof), any hydrophobic or hydrophilic bleach activator (e.g., dodecanoyl oxybenzene sulfonate, decanoyl oxybenzene sulfonate, decanoyl oxybenzoic acid or salts thereof, 3,5,5-trimethy hexanoyl oxybenzene sulfonate, tetraacetyl ethylene diamine-TAED, nonanoyloxybenzene sulfonate-NOBS, nitrile quats, and mixtures thereof), any source of hydrogen peroxide (e.g., inorganic perhydrate salts, examples of which include mono or tetra hydrate sodium salt of perborate, percarbonate, persulfate, perphosphate, or persilicate), any preformed hydrophilic and/or hydrophobic peracids (e.g., percarboxylic acids and salts, percarbonic acids and salts, perimidic acids and salts, peroxymonosulfuric acids and salts, and mixtures thereof); and/or (iv) any other components such as a bleach catalyst (e.g., imine bleach boosters examples of which include iminium cations and polyions, iminium zwitterions, modified amines, modified amine oxides, N-sulphonyl imines, N-phosphonyl imines, N-acyl imines, thiadiazole dioxides, perfluoroimines, cyclic sugar ketones, and mixtures thereof), and a metal-containing bleach catalyst (e.g., copper, iron, titanium, ruthenium, tungsten, molybdenum, or manganese cations along with an auxiliary metal cations such as zinc or aluminum and a sequestrate such as EDTA, ethylenediaminetetra(methylenephosphonic acid).

Compositions disclosed herein can be in the form of a dishwashing detergent composition, for example. Examples of dishwashing detergents include automatic dishwashing detergents (typically used in dishwasher machines) and hand-washing dish detergents. A dishwashing detergent composition can be in any dry or liquid/aqueous form as disclosed herein, for example. Components that may be included in certain embodiments of a dishwashing detergent composition include, for example, one or more of a phosphate; oxygen-or chlorine-based bleaching agent; non-ionic surfactant; alkaline salt (e.g., metasilicates, alkali metal hydroxides, sodium carbonate); any active enzyme disclosed herein; anti-corrosion agent (e.g., sodium silicate); anti-foaming agent; additives to slow down the removal of glaze and patterns from ceramics; perfume; anti-caking agent (in granular detergent); starch (in tablet-based detergents); gelling agent (in liquid/gel based detergents); and/or sand (powdered detergents).

Dishwashing detergents such as an automatic dishwasher detergent or liquid dishwashing detergent can comprise (i) a non-ionic surfactant, including any ethoxylated non-ionic surfactant, alcohol alkoxylated surfactant, epoxy-capped poly(oxyalkylated) alcohol, or amine oxide surfactant present in an amount from 0 to 10 wt %; (ii) a builder, in the range of about 5-60 wt %, including any phosphate builder (e.g., mono-phosphates, di-phosphates, tri-polyphosphates, other oligomeric-polyphosphates, sodium tripolyphosphate-STPP), any phosphate-free builder (e.g., amino acid-based compounds including methyl-glycine-diacetic acid [MGDA] and salts or derivatives thereof, glutamic-N, N-diacetic acid [GLDA] and salts or derivatives thereof, iminodisuccinic acid (IDS) and salts or derivatives thereof, carboxy methyl inulin and salts or derivatives thereof, nitrilotriacetic acid [NTA], diethylene triamine penta acetic acid [DTPA], B-alaninediacetic acid [B-ADA] and salts thereof), homopolymers and copolymers of poly-carboxylic acids and partially or completely neutralized salts thereof, monomeric polycarboxylic acids and hydroxycarboxylic acids and salts thereof in the range of 0.5 wt % to 50 wt %, or sulfonated/carboxylated polymers in the range of about 0.1 wt % to about 50 wt %; (iii) a drying aid in the range of about 0.1 wt % to about 10 wt % (e.g., polyesters, especially anionic polyesters, optionally together with further monomers with 3 to 6 functionalities-typically acid, alcohol or ester functionalities which are conducive to polycondensation, polycarbonate-, polyurethane-and/or polyurea-polyorganosiloxane compounds or precursor compounds thereof, particularly of the reactive cyclic carbonate and urea type); (iv) a silicate in the range from about 1 wt % to about 20 wt % (e.g., sodium or potassium silicates such as sodium disilicate, sodium meta-silicate and crystalline phyllosilicates); (v) an inorganic bleach (e.g., perhydrate salts such as perborate, percarbonate, perphosphate, persulfate and persilicate salts) and/or an organic bleach (e.g., organic peroxyacids such as diacyl-and tetraacylperoxides, especially diperoxydodecanedioic acid, diperoxytetradecanedioic acid, and diperoxyhexadecanedioic acid); (vi) a bleach activator (e.g., organic peracid precursors in the range from about 0.1 wt % to about 10 wt %) and/or bleach catalyst (e.g., manganese triazacyclononane and related complexes; Co, Cu, Mn, and Fe bispyridylamine and related complexes; and pentamine acetate cobalt(III) and related complexes); (vii) a metal care agent in the range from about 0.1 wt % to 5 wt % (e.g., benzatriazoles, metal salts and complexes, and/or silicates); and/or (viii) any active enzyme disclosed herein in the range from about 0.01 to 5.0 mg of active enzyme per gram of automatic dishwashing detergent composition, and an enzyme stabilizer component (e.g., oligosaccharides, polysaccharides, and inorganic divalent metal salts).

Compositions disclosed herein can be in the form of an oral care composition, for example. Examples of oral care compositions include dentifrices, toothpaste, mouth wash, mouth rinse, chewing gum, and edible strips that provide some form of oral care (e.g., treatment or prevention of cavities [dental caries], gingivitis, plaque, tartar, and/or periodontal disease). An oral care composition can also be for treating an “oral surface”, which encompasses any soft or hard surface within the oral cavity including surfaces of the tongue, hard and soft palate, buccal mucosa, gums and dental surfaces. A “dental surface” herein is a surface of a natural tooth or a hard surface of artificial dentition including a crown, cap, filling, bridge, denture, or dental implant, for example.

An oral care composition herein can comprise about 0.01-15.0 wt % (e.g., ˜0.1-10 wt % or ˜0.1-5.0 wt %, ˜0.1-2.0 wt %) of one or more graft copolymers as disclosed herein, for example. One or more graft copolymers comprised in an oral care composition can sometimes be provided therein as a thickening agent and/or dispersion agent, which may be useful to impart a desired consistency and/or mouth feel to the composition. One or more other thickening or dispersion agents can also be provided in an oral care composition herein, such as a carboxyvinyl polymer, carrageenan (e.g., L-carrageenan), natural gum (e.g., karaya, xanthan, gum arabic, tragacanth), colloidal magnesium aluminum silicate, or colloidal silica, for example.

An oral care composition herein may be a toothpaste or other dentifrice, for example. Such compositions, as well as any other oral care composition herein, can additionally comprise, without limitation, one or more of an anticaries agent, antimicrobial or antibacterial agent, anticalculus or tartar control agent, surfactant, abrasive, pH-modifying agent, foam modulator, humectant, flavorant, sweetener, pigment/colorant, whitening agent, and/or other suitable components. Examples of oral care compositions to which one or more graft copolymers can be added are disclosed in U.S. Patent Appl. Publ. Nos. 2006/0134025, 2002/0022006 and 2008/0057007, which are incorporated herein by reference.

An anticaries agent herein can be an orally acceptable source of fluoride ions. Suitable sources of fluoride ions include fluoride, monofluorophosphate and fluorosilicate salts as well as amine fluorides, including olaflur (N′-octadecyltrimethylendiamine-N,N,N′-tris(2-ethanol)-dihydrofluoride), for example. An anticaries agent can be present in an amount providing a total of about 100-20000 ppm, about 200-5000 ppm, or about 500-2500 ppm, fluoride ions to the composition, for example. In oral care compositions in which sodium fluoride is the sole source of fluoride ions, an amount of about 0.01-5.0 wt %, about 0.05-1.0 wt %, or about 0.1-0.5 wt %, sodium fluoride can be present in the composition, for example.

An antimicrobial or antibacterial agent suitable for use in an oral care composition herein includes, for example, phenolic compounds (e.g., 4-allylcatechol; p-hydroxybenzoic acid esters such as benzylparaben, butylparaben, ethylparaben, methylparaben and propylparaben; 2-benzylphenol; butylated hydroxyanisole; butylated hydroxytoluene; capsaicin; carvacrol; creosol; eugenol; guaiacol; halogenated bisphenolics such as hexachlorophene and bromochlorophene; 4-hexylresorcinol; 8-hydroxyquinoline and salts thereof; salicylic acid esters such as menthyl salicylate, methyl salicylate and phenyl salicylate; phenol; pyrocatechol; salicylanilide; thymol; halogenated diphenylether compounds such as triclosan and triclosan monophosphate), copper (II) compounds (e.g., copper (II) chloride, fluoride, sulfate and hydroxide), zinc ion sources (e.g., zinc acetate, citrate, gluconate, glycinate, oxide, and sulfate), phthalic acid and salts thereof (e.g., magnesium monopotassium phthalate), hexetidine, octenidine, sanguinarine, benzalkonium chloride, domiphen bromide, alkylpyridinium chlorides (e.g. cetylpyridinium chloride, tetradecylpyridinium chloride, N-tetradecyl-4-ethylpyridinium chloride), iodine, sulfonamides, bisbiguanides (e.g., alexidine, chlorhexidine, chlorhexidine digluconate), piperidino derivatives (e.g., delmopinol, octapinol), magnolia extract, grapeseed extract, rosemary extract, menthol, geraniol, citral, eucalyptol, antibiotics (e.g., augmentin, amoxicillin, tetracycline, doxycycline, minocycline, metronidazole, neomycin, kanamycin, clindamycin), and/or any antibacterial agents disclosed in U.S. Pat. No. 5,776,435, which is incorporated herein by reference. One or more antimicrobial agents can optionally be present at about 0.01-10 wt % (e.g., 0.1-3 wt %), for example, in the disclosed oral care composition.

An anticalculus or tartar control agent suitable for use in an oral care composition herein includes, for example, phosphates and polyphosphates (e.g., pyrophosphates), polyaminopropanesulfonic acid (AMPS), zinc citrate trihydrate, polypeptides (e.g., polyaspartic and polyglutamic acids), polyolefin sulfonates, polyolefin phosphates, diphosphonates (e.g., azacycloalkane-2,2-diphosphonates such as azacycloheptane-2,2-diphosphonic acid), N-methyl azacyclopentane-2,3-diphosphonic acid, ethane-1-hydroxy-1,1-diphosphonic acid (EHDP), ethane-1-amino-1,1-diphosphonate, and/or phosphonoalkane carboxylic acids and salts thereof (e.g., their alkali metal and ammonium salts). Useful inorganic phosphate and polyphosphate salts include, for example, monobasic, dibasic and tribasic sodium phosphates, sodium tripolyphosphate, tetrapolyphosphate, mono-, di-, tri-and tetra-sodium pyrophosphates, disodium dihydrogen pyrophosphate, sodium trimetaphosphate, sodium hexametaphosphate, or any of these in which sodium is replaced by potassium or ammonium. Other useful anticalculus agents in certain embodiments include anionic polycarboxylate polymers (e.g., polymers or copolymers of acrylic acid, methacrylic, and maleic anhydride such as polyvinyl methyl ether/maleic anhydride copolymers). Still other useful anticalculus agents include sequestering agents such as hydroxycarboxylic acids (e.g., citric, fumaric, malic, glutaric and oxalic acids and salts thereof) and aminopolycarboxylic acids (e.g., EDTA). One or more anticalculus or tartar control agents can optionally be present at about 0.01-50 wt % (e.g., about 0.05-25 wt % or about 0.1-15 wt %), for example, in the disclosed oral care composition.

A surfactant suitable for use in an oral care composition herein may be anionic, non-ionic, or amphoteric, for example. Suitable anionic surfactants include, without limitation, water-soluble salts of C₈₋₂₀ alkyl sulfates, sulfonated monoglycerides of C₈₋₂₀ fatty acids, sarcosinates, and taurates. Examples of anionic surfactants include sodium lauryl sulfate, sodium coconut monoglyceride sulfonate, sodium lauryl sarcosinate, sodium lauryl isoethionate, sodium laureth carboxylate and sodium dodecyl benzenesulfonate. Suitable non-ionic surfactants include, without limitation, poloxamers, polyoxyethylene sorbitan esters, fatty alcohol ethoxylates, alkylphenol ethoxylates, tertiary amine oxides, tertiary phosphine oxides, and dialkyl sulfoxides. Suitable amphoteric surfactants include, without limitation, derivatives of C₈₋₂₀ aliphatic secondary and tertiary amines having an anionic group such as a carboxylate, sulfate, sulfonate, phosphate or phosphonate. An example of a suitable amphoteric surfactant is cocoamidopropyl betaine. One or more surfactants are optionally present in a total amount of about 0.01-10 wt % (e.g., about 0.05-5.0 wt % or about 0.1-2.0 wt %), for example, in the disclosed oral care composition.

An abrasive suitable for use in an oral care composition herein may include, for example, silica (e.g., silica gel, hydrated silica, precipitated silica), alumina, insoluble phosphates, calcium carbonate, and resinous abrasives (e.g., a urea-formaldehyde condensation product). Examples of insoluble phosphates useful as abrasives herein are orthophosphates, polymetaphosphates and pyrophosphates, and include dicalcium orthophosphate dihydrate, calcium pyrophosphate, beta-calcium pyrophosphate, tricalcium phosphate, calcium polymetaphosphate and insoluble sodium polymetaphosphate. One or more abrasives are optionally present in a total amount of about 5-70 wt % (e.g., about 10-56 wt % or about 15-30 wt %), for example, in the disclosed oral care composition. The average particle size of an abrasive in certain embodiments is about 0.1-30 microns (e.g., about 1-20 microns or about 5-15 microns).

An oral care composition in certain embodiments may comprise at least one pH-modifying agent. Such agents may be selected to acidify, make more basic, or buffer the pH of a composition to a pH range of about 2-10 (e.g., pH ranging from about 2-8, 3-9, 4-8, 5-7, 6-10, or 7-9). Examples of pH-modifying agents useful herein include, without limitation, carboxylic, phosphoric and sulfonic acids; acid salts (e.g., monosodium citrate, disodium citrate, monosodium malate); alkali metal hydroxides (e.g. sodium hydroxide, carbonates such as sodium carbonate, bicarbonates, sesquicarbonates); borates; silicates; phosphates (e.g., monosodium phosphate, trisodium phosphate, pyrophosphate salts); and imidazole.

A foam modulator suitable for use in an oral care composition herein may be a polyethylene glycol (PEG), for example. High molecular weight PEGs are suitable, including those having an average molecular weight of about 200000-7000000 (e.g., about 500000-5000000 or about 1000000-2500000), for example. One or more PEGs are optionally present in a total amount of about 0.1-10 wt % (e.g. about 0.2-5.0 wt % or about 0.25-2.0 wt %), for example, in the disclosed oral care composition.

An oral care composition in certain embodiments may comprise at least one humectant. A humectant in certain embodiments may be a polyhydric alcohol such as glycerin, sorbitol, xylitol, or a low molecular weight PEG. Most suitable humectants also may function as a sweetener herein. One or more humectants are optionally present in a total amount of about 1.0-70 wt % (e.g., about 1.0-50 wt %, about 2-25 wt %, or about 5-15 wt %), for example, in the disclosed oral care composition.

A natural or artificial sweetener may optionally be comprised in an oral care composition herein. Examples of suitable sweeteners include dextrose, sucrose, maltose, dextrin, invert sugar, mannose, xylose, ribose, fructose, levulose, galactose, corn syrup (e.g., high fructose corn syrup or corn syrup solids), partially hydrolyzed starch, hydrogenated starch hydrolysate, sorbitol, mannitol, xylitol, maltitol, isomalt, aspartame, neotame, saccharin and salts thereof, dipeptide-based intense sweeteners, and cyclamates. One or more sweeteners are optionally present in a total amount of about 0.005-5.0 wt %, for example, in the disclosed oral care composition.

A natural or artificial flavorant may optionally be comprised in an oral care composition herein. Examples of suitable flavorants include vanillin; sage; marjoram; parsley oil; spearmint oil; cinnamon oil; oil of wintergreen (methylsalicylate); peppermint oil; clove oil; bay oil; anise oil; eucalyptus oil; citrus oils; fruit oils; essences such as those derived from lemon, orange, lime, grapefruit, apricot, banana, grape, apple, strawberry, cherry, or pineapple; bean-and nut-derived flavors such as coffee, cocoa, cola, peanut, or almond; and adsorbed and encapsulated flavorants. Also encompassed within flavorants herein are ingredients that provide fragrance and/or other sensory effect in the mouth, including cooling or warming effects. Such ingredients include, without limitation, menthol, menthyl acetate, menthyl lactate, camphor, eucalyptus oil, eucalyptol, anethole, eugenol, cassia, oxanone, Irisone®, propenyl guaiethol, thymol, linalool, benzaldehyde, cinnamaldehyde, N-ethyl-p-menthan-3-carboxamine, N,2,3-trimethyl-2-isopropylbutanamide, 3-(1-menthoxy)-propane-1,2-diol, cinnamaldehyde glycerol acetal (CGA), and menthone glycerol acetal (MGA). One or more flavorants are optionally present in a total amount of about 0.01-5.0 wt % (e.g., about 0.1-2.5 wt %), for example, in the disclosed oral care composition.

An oral care composition in certain embodiments may comprise at least one bicarbonate salt. Any orally acceptable bicarbonate can be used, including alkali metal bicarbonates such as sodium or potassium bicarbonate, and ammonium bicarbonate, for example. One or more bicarbonate salts are optionally present in a total amount of about 0.1-50 wt % (e.g., about 1-20 wt %), for example, in the disclosed oral care composition.

An oral care composition in certain embodiments may comprise at least one whitening agent and/or colorant. A suitable whitening agent is a peroxide compound such as any of those disclosed in U.S. Pat. No. 8,540,971, which is incorporated herein by reference. Suitable colorants herein include pigments, dyes, lakes and agents imparting a particular luster or reflectivity such as pearling agents, for example. Specific examples of colorants useful herein include talc; mica; magnesium carbonate; calcium carbonate; magnesium silicate; magnesium aluminum silicate; silica; titanium dioxide; zinc oxide; red, yellow, brown and black iron oxides; ferric ammonium ferrocyanide; manganese violet; ultramarine; titaniated mica; and bismuth oxychloride. One or more colorants are optionally present in a total amount of about 0.001-20 wt % (e.g., about 0.01-10 wt % or about 0.1-5.0 wt %), for example, in the disclosed oral care composition.

Additional components that can optionally be included in an oral composition herein include one or more enzymes (above), vitamins, and anti-adhesion agents, for example. Examples of vitamins useful herein include vitamin C, vitamin E, vitamin B5, and folic acid. Examples of suitable anti-adhesion agents include solbrol, ficin, and quorum-sensing inhibitors.

The present disclosure also concerns a method of treating a material. This method comprises contacting a material with an aqueous composition comprising at least one graft copolymer as disclosed herein.

A material contacted with an aqueous composition in a contacting method herein can comprise a fabric in certain embodiments. A fabric herein can comprise natural fibers, synthetic fibers, semi-synthetic fibers, or any combination thereof. A semi-synthetic fiber herein is produced using naturally occurring material that has been chemically derivatized, an example of which is rayon. Non-limiting examples of fabric types herein include fabrics made of (i) cellulosic fibers such as cotton (e.g., broadcloth, canvas, chambray, chenille, chintz, corduroy, cretonne, damask, denim, flannel, gingham, jacquard, knit, matelassé, oxford, percale, poplin, plissé, sateen, seersucker, sheers, terry cloth, twill, velvet), rayon (e.g., viscose, modal, lyocell), linen, and Tencel®; (ii) proteinaceous fibers such as silk, wool and related mammalian fibers; (iii) synthetic fibers such as polyester, acrylic, nylon, and the like; (iv) long vegetable fibers from jute, flax, ramie, coir, kapok, sisal, henequen, abaca, hemp and sunn; and (v) any combination of a fabric of (i)-(iv). Fabric comprising a combination of fiber types (e.g., natural and synthetic) include those with both a cotton fiber and polyester, for example. Materials/articles containing one or more fabrics herein include, for example, clothing, curtains, drapes, upholstery, carpeting, bed linens, bath linens, tablecloths, sleeping bags, tents, car interiors, etc. Other materials comprising natural and/or synthetic fibers include, for example, non-woven fabrics, paddings, paper, and foams.

An aqueous composition that is contacted with a fabric can be, for example, a fabric care composition (e.g., laundry detergent, fabric softener). Thus, a treatment method in certain embodiments can be considered a fabric care method or laundry method if employing a fabric care composition therein. A fabric care composition herein is contemplated to effect one or more of the following fabric care benefits (i.e., surface substantive effects): wrinkle removal, wrinkle reduction, wrinkle resistance, fabric wear reduction, fabric wear resistance, fabric pilling reduction, extended fabric life, fabric color maintenance, fabric color fading reduction, reduced dye transfer, fabric color restoration, fabric soiling reduction, fabric soil release, fabric shape retention, fabric smoothness enhancement, anti-redeposition of soil on fabric, anti-greying of laundry, improved fabric hand/handle, and/or fabric shrinkage reduction.

Examples of conditions (e.g., time, temperature, wash/rinse volumes) for conducting a fabric care method or laundry method herein are disclosed in WO1997/003161 and U.S. Pat. Nos. 4,794,661, 4,580,421 and 5,945,394, which are incorporated herein by reference.

In other examples, a material comprising fabric can be contacted with an aqueous composition herein: (i) for at least about 5, 10, 20, 30, 40, 50, 60, 70, 80, 90, 100, 110, or 120 minutes; (ii) at a temperature of at least about 10, 15, 20, 25, 30, 35, 40, 45, 50, 55, 60, 65, 70, 75, 80, 85, 90, or 95° C. (e.g., for laundry wash or rinse: a “cold” temperature of about 15-30° C., a “warm” temperature of about 30-50° C., a “hot” temperature of about 50-95° C.); (iii) at a pH of about 2, 3, 4, 5, 6, 7, 8, 9, 10, 11, or 12 (e.g., pH range of about 2-12, or about 3-11); (iv) at a salt (e.g., NaCl) concentration of at least about 0.5, 1.0, 1.5, 2.0, 2.5, 3.0, 3.5, or 4.0 wt %; or any combination of (i)-(iv).

The contacting step in a fabric care method or laundry method can comprise any of washing, soaking, and/or rinsing steps, for example. Contacting a material or fabric in still further embodiments can be performed by any means known in the art, such as dissolving, mixing, shaking, spraying, treating, immersing, flushing, pouring on or in, combining, painting, coating, applying, affixing to, and/or communicating an effective amount of a graft copolymer herein with the fabric or material. In still further embodiments, contacting may be used to treat a fabric to provide a surface substantive effect. As used herein, the term “fabric hand” or “handle” refers to a person's tactile sensory response towards fabric which may be physical, physiological, psychological, social or any combination thereof. In one embodiment, the fabric hand may be measured using a PhabrOmeter® System for measuring relative hand value (available from Nu Cybertek, Inc. Davis, CA) (American Association of Textile Chemists and Colorists [AATCC test method “202-2012, Relative Hand Value of Textiles: Instrumental Method”]).

In certain embodiments of treating a material comprising fabric, a graft copolymer component(s) of the aqueous composition adsorbs to the fabric. This feature is believed to render graft copolymers herein useful as anti-redeposition agents and/or anti-greying agents in fabric care compositions disclosed (in addition to their viscosity-modifying effect). An anti-redeposition agent or anti-greying agent herein helps keep soil from redepositing onto clothing in wash water after the soil has been removed. It is further contemplated that adsorption of one or more graft copolymers herein to a fabric enhances mechanical properties of the fabric.

Adsorption of a graft copolymer to a fabric herein can be measured using a colorimetric technique (e.g., Dubois et al., 1956, Anal. Chem. 28:350-356; Zemljič et al., 2006, Lenzinger Berichte 85:68-76; both incorporated herein by reference), for example, or any other method known in the art.

Other materials that can be contacted in the above treatment method include surfaces that can be treated with a dish detergent (e.g., automatic dishwashing detergent or hand dish detergent). Examples of such materials include surfaces of dishes, glasses, pots, pans, baking dishes, utensils and flatware made from ceramic material, china, metal, glass, plastic (e.g., polyethylene, polypropylene, polystyrene, etc.) and wood (collectively referred to herein as “tableware”). Thus, the treatment method in certain embodiments can be considered a dishwashing method or tableware washing method, for example. Examples of conditions (e.g., time, temperature, wash volume) for conducting a dishwashing or tableware washing method herein are disclosed in U.S. Pat. No. 8,575,083, which is incorporated herein by reference. In other examples, a tableware article can be contacted with an aqueous composition herein under a suitable set of conditions such as any of those disclosed above with regard to contacting a fabric-comprising material.

Other materials that can be contacted in the above treatment method include oral surfaces such as any soft or hard surface within the oral cavity including surfaces of the tongue, hard and soft palate, buccal mucosa, gums and dental surfaces (e.g., natural tooth or a hard surface of artificial dentition such as a crown, cap, filling, bridge, denture, or dental implant). Thus, a treatment method in certain embodiments can be considered an oral care method or dental care method, for example. Conditions (e.g., time, temperature) for contacting an oral surface with an aqueous composition herein should be suitable for the intended purpose of making such contact. Other surfaces that can be contacted in a treatment method also include a surface of the integumentary system such as skin, hair or nails.

Thus, certain embodiments of the present disclosure concern material (e.g., fabric) that comprises a graft copolymer herein. Such material can be produced following a material treatment method as disclosed herein, for example. A material can comprise a graft copolymer in certain aspects if the copolymer is adsorbed to, or otherwise in contact with, the surface of the material.

Certain embodiments of a method of treating a material herein further comprise a drying step, in which a material is dried after being contacted with the aqueous composition. A drying step can be performed directly after the contacting step, or following one or more additional steps that might follow the contacting step (e.g., drying of a fabric after being rinsed, in water for example, following a wash in an aqueous composition herein). Drying can be performed by any of several means known in the art, such as air drying (e.g., ˜20-25° C.), or at a temperature of at least about 30, 40, 50, 60, 70, 80, 90, 100, 120, 140, 160, 170, 175, 180, or 200° C., for example. A material that has been dried herein typically has less than 3, 2, 1, 0.5, or 0.1 wt % water comprised therein. Fabric is a preferred material for conducting an optional drying step.

An aqueous composition used in a treatment method herein can be any aqueous composition disclosed herein, such as in the above embodiments. Thus, the graft copolymer component(s) of an aqueous composition can be any as disclosed herein. Examples of aqueous compositions include detergents (e.g., laundry detergent or dish detergent) and water-containing dentifrices such as toothpaste.

Non-limiting examples of compositions and methods disclosed herein include:

1. A composition comprising a graft copolymer that comprises: (i) a backbone comprising an alpha-1,3-glucan ether or ester compound that has a degree of substitution (DoS) of about 0.001 to about 3.0, and (ii) one or more alpha-1,3-glucan side chains comprising at least about 50% alpha-1,3 glycosidic linkages.
2. The composition of embodiment 1, wherein the alpha-1,3-glucan ether or ester compound comprises at least about 50% alpha-1,3 glycosidic linkages.
3. The composition of embodiment 1 or 2, wherein the weight-average degree of polymerization (DPw) of the alpha-1,3-glucan ether or ester compound is at least about 15.
4. The composition of embodiment 1, 2, or 3, wherein the backbone comprises an alpha-1,3-glucan ether compound.
5. The composition of embodiment 1, 2, 3, or 4, wherein the alpha-1,3-glucan ether compound comprises an ether-linked group that is anionic or cationic when the compound is comprised in an aqueous composition.
6. The composition of embodiment 1, 2, 3, 4, or 5, wherein the alpha-1,3-glucan ether compound comprises an ether-linked carboxymethyl group.
7. The composition of embodiment 1, 2, 3, 4, 5, or 6, wherein: (a) the alpha-1,3-glucan side chains comprise at least about 90% alpha-1,3 glycosidic linkages, and/or (b) the DP or DPw of the one or more alpha-1,3-glucan side chains is at least about 100.
8. The composition of embodiment 1, 2, 3, 4, 5, 6, or 7, wherein the composition is an aqueous composition, optionally wherein the graft copolymer is insoluble in the aqueous composition.
9. The composition of embodiment 8, wherein aqueous composition is a dispersion of the graft copolymer, optionally wherein the dispersion comprises less than 1.5 w/v % of the graft copolymer.
10. The composition of embodiment 8 or 9, wherein: (a) the zeta potential of the graft copolymer in the aqueous composition is over ±15 mV, and/or (b) the particle size of the graft copolymer in the aqueous composition is less than 1 micron.
11. The composition of embodiment 1, 2, 3, 4, 5, 6, 7, 8, 9, or 10, wherein the composition is a household care product, personal care product, industrial product, pharmaceutical product, or food product.
12. The composition of embodiment 1, 2, 3, 4, 5, 6, 7, 8, 9, 10, or 11, wherein the graft copolymer is produced in a reaction composition comprising at least water, sucrose, the alpha-1,3-glucan ether or ester compound, and a glucosyltransferase enzyme that synthesizes alpha-1,3-glucan with at least about 50% alpha-1,3 glycosidic linkages, optionally wherein the composition is said reaction composition.
13. A method of producing a graft copolymer, the method comprising: (a) contacting, in a reaction composition, at least (i) water, (ii) sucrose, (iii) an alpha-1,3-glucan ether or ester compound that has a degree of substitution (DoS) of about 0.001 to about 3.0, and (iv) a glucosyltransferase enzyme that synthesizes alpha-1,3-glucan comprising at least about 50% alpha-1,3 glycosidic linkages, whereby a graft copolymer according to the composition of embodiment 1, 2, 3, 4, 5, 6, 7, 8, 9, or 10 is produced, optionally wherein the viscosity of the reaction composition increases by at least 10% at least 1 hour following the contacting step; and (b) optionally, isolating the graft copolymer produced in step (a).
14. A method of providing an aqueous composition, the method comprising: (a) providing a graft copolymer according to the composition of embodiment 1, 2, 3, 4, 5, 6, 7, 8, 9, or 10, and (b) dispersing or dissolving the graft copolymer into an aqueous liquid, thereby producing the aqueous composition.
15. The method of embodiment 14, wherein the graft copolymer provided in step (a) is dry.
16. The method of embodiment 14 or 15, wherein the graft copolymer provided in step (a) is insoluble in aqueous conditions, and wherein the graft copolymer is dispersed into the aqueous liquid in step (b).
17. The method of embodiment 14, 15, or 16, wherein the aqueous composition produced in step (b) comprises less than 1.5 w/v % of the graft copolymer.
18. The method of embodiment 14, 15, 16, or 17, wherein the viscosity of the aqueous composition produced in step (b) is at least 10% higher than the viscosity of the aqueous liquid.

### EXAMPLES

The present disclosure is further exemplified in the below Examples. It should be understood that these Examples, while indicating certain aspects herein, are given by way of illustration only. From the above discussion and these Examples, one skilled in the art can ascertain the essential characteristics of the disclosed embodiments, and without departing from the spirit and scope thereof, can make various changes and modifications to adapt the disclosed embodiments to various uses and conditions.

### General Methods (Aside Those Listed in the Below Examples)

### Analysis of Glucan Molecular Weight by Size-Exclusion Chromatography (SEC)

Insoluble glucan polymer wet cake isolated from glucosyltransferase reactions was dissolved at 2 mg/mL with overnight shaking in neat DMSO with 2% lithium chloride (LiCl) from Aldrich (Milwaukee, WI) to form a glucan polymer solution. This solution (100 μL, 40° C.) was then injected into an Alliance™ 2695 HPLC (Waters Corporation, Milford, MA) coupled with three on-line detectors: a differential refractometer (DR) 2414™ from Waters Corp., a multiangle light-scattering photometer Heleos II™ 18 angles from Wyatt Technologies (Santa Barbara, CA), and a differential capillary viscometer ViscoStar™ from Wyatt Tech, all operating at 50° C. The mobile phase (DMAc containing 0.11 wt % LiCl) passed at a flow rate of 0.5 mL/min through four styrene-divinyl benzene columns in series; specifically, one KD-802, one KD-801, and two linear KD-806M columns (Shodex, Japan). The molecular weight distribution of the glucan polymer sample, as well as its average molar masses (Mn, Mw and Mz) were determined using Astra version 7.1 software package from Wyatt (triple detection method without column calibration). Values of average degree of polymerization, e.g., DPw, were calculated by dividing a corresponding average molar mass by 162. This SEC protocol yielded the following glucan polymer measurements: average molecular weights, molecular weight distribution, intrinsic viscosity, Mark-Houwink and conformation plots, radius of gyration, branching frequency. The DPw and PDI values of all graft copolymer products disclosed herein include the DPw/PDI values of both the glucan ether primer and the alpha-1,3-glucan synthesized off the primer.

### Determination of Glycosidic Linkages

Glycosidic linkages in glucan products synthesized by a glucosyltransferase were determined by ¹H NMR (nuclear magnetic resonance) spectroscopy. Dry glucan polymer (6 to 8 mg) was dissolved in 0.75 mL of 3 wt % lithium chloride (LiCl) in deuterated dimethyl sulfoxide (DMSO-d6) by stirring overnight at ambient temperature. Deuterated water (D₂O) was then added (0.05 mL), and the sample was heated at 80° C. for about one hour to exchange protonated hydroxyls on the glucan polymer and ensure complete dissolution. 600 μL of the resulting clear homogeneous solution was transferred to a 5-mm NMR tube. ¹H NMR spectra was used to quantify glycosidic linkage and a 2D ¹H, ¹³C homo/hetero-nuclear suite of experiments was used to identify glucan linkages. The data were collected at 80° C. and processed on a Bruker Avance III NMR spectrometer, operating at either 500 MHz or 600 MHz. The systems are equipped with a proton-optimized cryoprobe.

### Percent Primer Incorporation

The percent incorporation of primer herein was determined using ¹H NMR (nuclear magnetic resonance) spectroscopy measurements. Dry glucan product was first fully depolymerized to monomeric anhydroglucose (AGU) units by sulfuric acid hydrolysis. Approximately 20 mg of polymer was dissolved by stirring in 0.175 ml of a 60% deuterated sulfuric acid (D₂SO₄) solution in 99.99% deuterated water (D₂O) at ambient temperatures until clear (typically about 1 hour). The homogenous solution was diluted to 12% D₂SO₄ by adding 0.6 mL of D₂O and 0.1 mL of D₂O containing 3-(trimethylsilyl)-1-propanesulfonic acid sodium salt (DSS) used as an internal NMR chemical shift standard. The sample was then moved to a stirring heat block set at 90° C. for 2 hours. The resulting pale-yellow solution was transferred to a 5-mm NMR tube. Quantitative 1-D ¹H NMR data were then collected on a Bruker DRX at 500 MHz or Bruker Avance III at 600 MHZ. In general, the degree of substitution (DoS) of the CMG (carboxymethyl glucan) primer was determined to measure the number of moles AGU for CMG. DoS of CMG is defined as the moles of CM (carboxymethyl) divided by the moles of AGU. Moles of CMG in CMGPG (CMG-primed glucan) was determined as ratio of moles CM divided by the CMG primer DoS. The moles of grafted glucan (alpha-1,3-glucan side-chains/arms) was found from the difference of total moles AGU measured in the NMR of the CMGPG and moles AGU of CMG primer. Finally, the mol % CMG was determined from moles CMG divided by the total moles of AGU multiplied by 100. Specifically, the mol % CMG incorporation in CMGPG was determined from the NMR spectrum as follows:

$$Mol \% C M G = ( ( Moles C M G ) / ( total Moles A G U ) ) * 100.$$

The CMG CH₂ group exhibits a complex series of multiplets due to regioselectivity of the CM moiety on positions 2, 4, and/or 6 and anomeric configuration of the AGU.

$$Moles C M = \int CM / 2 = ( \int ( δ 4.53 ppm to δ 4.32 ppm ) + \int ( δ 4.27 ppm to δ 4.23 ppm ) ) / 2.$$

$$Moles C M G = Moles CM / DoS .$$

The mole ratio of AGU was measured as the sum of all observed α/β anomeric protons.

$$Moles A G U = \int ( δ5 \text{.44} ppm to δ4 \text{.57} ppm ) .$$

### Carboxymethyl Alpha-1,3-Glucan Ether (CMG)

Water-soluble CMG was prepared by entering unmodified alpha-1,3-glucan (˜100% alpha-1,3 glycosidic linkages) with a DPw of about 760 or 1220 into etherification reactions similar to, or the same as, the reactions disclosed in U.S. Patent Appl. Publ. Nos. 2016/0304629 and 2014/0179913, which are incorporated herein by reference. The etherification agent was sodium chloroacetate. CMG with a degree of substitution (DoS) with carboxymethyl groups of about 0.25, 0.31, 0.70, or 1.1 was prepared (respectively termed herein as CMG25, CMG31, CMG70 and CMG110).

### High Yielding Alpha-1,3-Glucan-Producing Glucosyltransferase Enzymes

The amino acid sequence of the glucosyltransferase used to prepare amino acid substitutions was SEQ ID NO:4 (GTF 6855), which essentially is an N-terminally truncated (signal peptide and variable region removed) version of the full-length wild type glucosyltransferase (represented by SEQ ID NO:62) from Streptococcus salivarius SK126 (see Table 1). Substitutions made in SEQ ID NO:4 can be characterized as substituting for native amino acid residues, as each amino acid residue/position of SEQ ID NO:4 (apart from the Met-1 residue of SEQ ID NO:4) corresponds accordingly with an amino acid residue/position within SEQ ID NO:62. In reactions comprising at least sucrose and water, the glucosyltransferase of SEQ ID NO:4 typically produces alpha-glucan having about 100% alpha-1,3 linkages and a DPw of 400 or greater (e.g., refer to U.S. Patent No. U.S. Patent Appl. Publ. No. 2017/0002335, which is incorporated herein by reference). This alpha-glucan product, which is insoluble, can be isolated following enzymatic synthesis via filtration, for example.

Briefly, certain combinations of amino acid substitutions were made to SEQ ID NO:4 (GTF 6855). These substitutions are listed in Tables A and B below. Each variant enzyme listed in Table A was entered into a glucan synthesis reaction with parameters that were the same as, or similar to, the following: vessel, 250-mL indented shake flask agitated at 120 rpm; initial pH, 5.7; reaction volume, 50 mL; sucrose, 75 g/L; GTF, 1.5 mL lysate of E. coli cells heterologously expressing enzyme; KH₂PO4, 20 mM; temperature, 30° C.; time, about 20-24 hours. The alpha-1,3-glucan yields of these reactions (measured by HPLC analysis) are provided in Table A.

TABLE A
Alpha-1,3-Glucan Yields of GTF 6855 (SEQ ID NO: 4)
Variants with Multiple Amino Acid Substitutions

| | Alpha-1,3- |
|---|---|
| GTF^{a} | Glucan^{b} Yield^{c} |
| A510D/F607Y/R741S | 72.6% |
| A510D/F607Y/N743S | 79.2% |
| A510D/F607Y/D948G | 88.2% |
| A510D/R741S/D948G | 74.5% |
| A510D/F607Y/R741S/D948G | 82.8% |
| A510E/F607Y/R741S/R1172C | 78.2% |
| A510D/F607Y/D820G/D948G | 87.8% |
| A510D/F607Y/D948G/R1172C | 88.6% |
| A510D/F607Y/N743S/D948G/R1172C | 89.4% |
| A510D/F607Y/R741S/L784Q/F929L/R1172C | 79.3% |

^{a}Each listed GTF is a version of GTF 6855 (SEQ ID NO: 4) comprising substitutions at respective positions, where each position number is in correspondence with the residue numbering of SEQ ID NO: 62. The wild type residue is listed first (before residue position number) and the substituting residue is listed second (after the residue position number).
^{b}Insoluble alpha-1,3-glucan product with 100% alpha-1,3 linkages.
^{c}Alpha-1,3-glucan yield based on glucosyl. The average yield of unmodified GTF 6855 (SEQ ID NO: 4, no substitutions) was about 29%.

Each variant enzyme listed in Table B was entered into a glucan synthesis reaction with parameters that were the same as, or similar to, the following: vessel, 500-mL jacketed reactor with Teflon®-pitched blade turbine (45-degree angle) on a glass stir rod and agitated at 50-200 rpm; initial pH, 5.5; reaction volume, 500 mL; sucrose, 108 g/L; KH₂PO₄, 1 mM; temperature, 39° C.; time, about 18-24 hours; filtrate from a previous alpha-1,3-glucan synthesis reaction, 50 vol %. The alpha-1,3-glucan yields of these reactions (measured by HPLC analysis) are provided in Table B.

TABLE B
Alpha-1,3 Glucan Yields of GTF 6855 (SEQ ID NO: 4) Variants with Multiple Amino Acid-Substitutions

| | | | | | | | | | | | | | Alpha-1,3-Glucan^{b} |
|---|---|---|---|---|---|---|---|---|---|---|---|---|---|
| GTF^{a} | | | | | | | | | | | | | Yield^{c} |
| A510D | Q588L | F607Y | R741S | D948G | | | R722H | T877K | | M1253I | K1277N | | 88% |
| A510D | Q588L | F607Y | R741S | D948G | | | R722H | T877K | V1188E | M1253I | Q957P | | 92% |
| A510D | Q588L | F607Y | R741S | D948G | | | | T877K | V1188E | M1253I | Q957P | | 91% |
| A510D | Q588L | F607Y | R741S | D948G | | | | | | M1253I | | | 89% |
| A510D | Q588L | F607W | R741S | D948G | | | | | | | | | 91% |
| | Q588L | F607Y | R741S | D948G | | | | | | | | | 91% |
| A510D | Q588L | F607Y | R741S | D948G | N628D | T635A | | T877K | | M1253I | F929L | R1172C | 92% |
| A510D | Q588L | F607W | R741S | D948G | S631T | S710G | R722H | T877K | V1188E | M1253I | | | 94% |
| A510D | Q588L | F607W | R741S | D948G | S631T | S710G | R722H | T877K | V1188E | | | | 93% |
| A510D | Q588L | F607W | R741S | D948G | S631T | S710G | | T877K | V1188E | M1253I | | | 96% |
| A510D | Q588L | F607Y | R741S | D948G | | | | | | | | | 89% |
| A510D | Q588L | F607Y | R741S | D948G | | | | | V1188E | | | | 88% |
| A510D | Q588L | F607W | R741S | D948G | S631T | S710G | | | V1188E | | | | 96% |
| A510D | Q588L | F607W | R741S | D948G | | S710G | R722H | T877K | | M1253I | | | 96% |
| A510D | Q588L | F607Y | R741S | D948G | S631T | | R722H | T877K | V1188E | M1253I | | | 96% |
| A510D | Q588L | F607W | R741S | D948G | S631T | | | T877K | V1188E | M1253I | | | 94% |
| A510D | Q588L | F607W | R741S | D948G | S631T | | | | V1188E | | | | 98% |
| A510D | Q588L | F607Y | R741S | D948G | S631T | | R722H | T877K | V1188E | M1253I | | | 95% |
| A510D | Q588L | F607W | R741S | D948G | | | | | V1188E | M1253I | | | 93% |

^{a}Each listed GTF is a version of GTF 6855 (SEQ ID NO: 4) comprising substitutions at respective positions, where each position number is in correspondence with the residue numbering of SEQ ID NO: 62.
^{b}Insoluble alpha-1,3 glucan product.
^{c}Alpha-1,3-glucan yield based on glucosyl.

### Glucosyltransferase Enzymes that Produce Lower Molecular Weight Alpha-1,3-Glucan

As discussed above, SEQ ID NO:4 (GTF 6855) is an amino acid sequence of a glucosyltransferase that can be used to prepare amino acid substitutions. Briefly, certain combinations of amino acid substitutions can be made to SEQ ID NO:4 to provide a glucosyltransferase that produces alpha-1,3-glucan of lower molecular weight (as compared to alpha-1,3-glucan product of non-modified GTF 6855). These substitutions are listed in Tables C and D below. For collecting the data in these tables, each variant enzyme was entered into a glucan synthesis reaction with parameters that were the same as, or similar to, the following: vessel, 50-mL indented shake flask agitated at 75 rpm; initial pH, 5.7;

reaction volume, 10 mL; sucrose, 400 g/L; GTF, 0.3 mL of culture supernatant (prepared from lysate of E. coli cells heterologously expressing enzyme); KH₂PO4, 5 mM; temperature, 35° C.; time, about two days; de-activation, heated at 80° C. for 30 minutes. Insoluble glucan polymers produced in the reactions were individually harvested, water-washed, and analyzed for molecular size (DPw) via a standard SEC approach (see Tables C and D for DPw data). A glucosyltransferase with any of the amino acid substitution(s) listed in Tables C and D is contemplated to be useful in practicing the presently disclosed subject matter.

TABLE C
DPw of Insoluble Alpha-1,3-Glucan Produced by GTF 6855 (SEQ
ID NO: 4) and Single Amino Acid-Substituted Variants thereof

| GTF | DPw | GTF | DPw | GTF | DPw |
|---|---|---|---|---|---|
| 6855^{a} | 350 | S553A | 127 | N573A | 123 |
| L513A^{b} | 194 | S553C | 125 | N573A | 125 |
| L513C | 119 | S553C | 126 | N573D | 108 |
| L513C | 159 | S553E | 105 | N573D | 134 |
| L513D | 147 | S553E | 122 | N573G | 126 |
| L513D | 640 | S553F^{c} | | N573G | 120 |
| L513E | 129 | S553F^{c} | | N573H | 148 |
| L513E | 130 | S553H^{c} | | N573I^{c} | |
| L513F | 171 | S553H^{c} | | N573K | 145 |
| L513G | 138 | S553I | 79 | N573K | 148 |
| L513H | 153 | S553I | 97 | N573L^{c} | |
| L513H | 175 | S553M | 129 | N573M^{c} | |
| L513I | 186 | S553M | 140 | N573N | 222 |
| L513K | 143 | S553N | 77 | N573P | 100 |
| L513K | 160 | S553N | 69 | N573T | 102 |
| L513M | 183 | S553R | 63 | N573T | 109 |
| L513M | 210 | S553T | 226 | N573V | 91 |
| L513N^{c} | | S553T | 124 | N573W | 249 |
| L513N | 372 | S553V | 86 | N573W | 237 |
| L513P | 173 | S553Y | 110 | | |
| L513Q | 138 | S553Y | 52 | | |
| L513Q | 152 | | | | |
| L513R | 134 | | | | |
| L513R | 141 | | | | |
| L513S | 138 | | | | |
| L513S | 152 | | | | |
| L513T | 146 | | | | |
| L513V | 175 | | | | |
| L513V^{c} | | | | | |
| L513W | 146 | | | | |
| L513W | 171 | | | | |
| L513Y | 156 | | | | |
| 6855^{a} | 350 | K578A | 110 | Q616A | 175 |
| D575A^{b} | 74 | K578A | 113 | Q616A | 440 |
| D575A | 199 | K578C | 132 | Q616C | 81 |
| D575C | 97 | K578D | 156 | Q616D | 115 |
| D575C^{c} | | K578E | 95 | Q616E | 50 |
| D575C | 94 | K578E^{c} | | Q616G | 66 |
| D575E | 90 | K578F | 103 | Q616G^{c} | |
| D575E | 88 | K578G | 113 | Q616H | 61 |
| D575F | 74 | K578G | 103 | Q616I | 82 |
| D575G | 90 | K578H | 212 | Q616K | 58 |
| D575G | 89 | K578H | 187 | Q616K | 59 |
| D575H | 70 | K578I | 179 | Q616L | 61 |
| D575H | 134 | K578L | 177 | Q616L | 62 |
| D575I | 76 | K578M | 135 | Q616M | 164 |
| D575I | 98 | K578M | 141 | Q616N | 269 |
| D575K | 52 | K578N | 185 | Q616N | 211 |
| D575K | 95 | K578P | 126 | Q616P | 75 |
| D575L | 74 | K578P | 128 | Q616P | 78 |
| D575L^{c} | | K578Q | 111 | Q616R | 103 |
| D575M | 66 | K578R | 214 | Q616R | 167 |
| D575M | 72 | K578R | 294 | Q616S | 72 |
| D575N | 90 | K578S | 105 | Q616T | 79 |
| D575N | 191 | K578S | 105 | Q616V | 88 |
| D575N^{c} | | K578T | 131 | Q616V | 97 |
| D575P | 50 | K578T | 157 | Q616W | 60 |
| D575R | 65 | K578V | 146 | Q616W | 101 |
| D575R | 71 | K578V | 145 | Q616Y | 65 |
| D575S | 104 | K578W | 106 | | |
| D575S | 96 | K578W | 122 | | |
| D575V | 54 | K578Y | 145 | | |
| D575W | 69 | | | | |
| D575W | 167 | | | | |
| D575Y | 124 | | | | |
| D575Y | 69 | | | | |

^{a}GTF 6855, SEQ ID NO: 4. The DPw of insoluble alpha-1,3-glucan produced by GTF 6855 averaged to be about 350.
^{b}Each listed GTF with a substitution is a version of GTF 6855 comprising a substitution at a respective position, where the position number is in correspondence with the residue numbering of SEQ ID NO: 62.
^{c}Insoluble alpha-1,3-glucan not produced or detected.

TABLE D
DPw of Insoluble Alpha-1,3-Glucan Produced by Multiple Amino
Acid-Substituted Variants of GTF 6855 (SEQ ID NO: 4)

| GTF^{a} | | | | | | DPw |
|---|---|---|---|---|---|---|
| P550L | N557I | N581P | | | | 12 |
| L535P | S553C | N558D | D575V | T585P | K697R | 12 |
| P550V | S553R | N581P | T585P | | | 12 |
| P550L | S553F | N581P | | | | 12 |
| P550V | N557E | T585P | | | | 12 |
| P550L | N557E | D575V | T585P | | | 13 |
| L538P | P550L | S553Y | | | | 13 |
| P544L | P550V | S553C | N573I | T585P | S589G | 13 |
| P550V | G576D | T585P | | | | 13 |
| P550L | N558D | T585P | T679I | | | 13 |
| P550L | N557E | T585P | S589G | | | 14 |
| P550L | N557E | T569L | N581P | | | 14 |
| P550L | N557E | T569L | T585P | | | 14 |
| P550V | S553T | N558D | T585P | G730D | | 14 |
| E577G | P550L | N557I | T569L | N573I | | 14 |
| P550L | S553C | D575A | T585P | S589G | | 14 |
| S553R | N573V | K578N | S631G | T660A | | 14 |
| P550V | S553R | W571V | G576D | | | 14 |
| P550V | N557E | K578D | T585P | | | 14 |
| P550V | N558D | N573P | T585P | | | 14 |
| P550L | N558D | W571V | N581P | K593E | | 14 |
| P550V | S553E | N581P | | | | 15 |
| P550L | N573I | T585P | W725R | | | 15 |
| P550L | N557I | N573P | | | | 15 |
| N557E | N573V | N581P | | | | 15 |
| P550L | N557I | G576D | Q643L | | | 15 |
| P550V | S553N | T585P | V586G | S710G | | 15 |
| P550V | S553C | D575A | T585P | | | 15 |
| S553R | N573V | K578N | S631G | T660A | | 15 |
| P550L | S553K | D575A | Y580H | | | 16 |
| P550V | D575A | T585P | S589G | K713E | | 16 |
| P550V | S553N | N573I | Y693C | | | 16 |
| P544L | P550L | N557E | N573I | T585P | | 16 |
| P550L | N558D | W571V | N581P | T585P | | 16 |
| S504G | P550V | N557Q | N581P | | | 16 |
| P550L | S553R | D575A | | | | 16 |
| P550V | N558D | W571D | D575A | T585P | | 16 |
| P544L | P550V | N557Q | N581P | | | 17 |
| P550V | S553K | T585P | | | | 17 |
| P550V | S553N | T585P | | | | 17 |
| P550L | T569L | N573I | | | | 17 |
| P550L | N558D | D575V | | | | 17 |
| L537P | P550L | N558D | N573I | | | 17 |
| P550L | S553C | W571C | G576D | T585P | | 17 |
| P550L | N557Q | W571C | G576D | T585P | | 17 |
| P550L | N558D | W571V | N581P | | | 17 |
| A542V | P550V | N558D | W571V | T585P | | 17 |
| P550V | N558D | W571D | G576D | | | 18 |
| P550V | S553N | N573I | | | | 18 |
| P550V | N557Q | D575V | A669T | | | 18 |
| P550V | N581P | I636T | | | | 18 |
| P550V | N557E | N581P | | | | 18 |
| P550V | N573I | T585P | | | | 18 |
| P550L | S553K | N558D | K578R | Y700N | | 19 |
| P550V | S553T | N558D | W571V | | | 19 |
| P514L | P550V | N557Q | T585P | D602N | | 20 |
| P550L | N557I | T569A | G576D | | | 20 |
| P550V | N557T | N558D | W571D | | | 21 |
| P550L | N557E | D575A | T585P | | | 21 |
| I545V | P550V | N557Q | T585P | D638N | | 21 |
| P544L | P550V | N557I | T585P | | | 22 |
| Y518C | P550V | N581P | T585P | | | 22 |
| P550L | N557E | D575A | | | | 22 |

^{a}Each listed GTF is a version of GTF 6855 (SEQ ID NO: 4) comprising substitutions at respective positions, where each position number is in correspondence with the residue numbering of SEQ ID NO: 62.

### Glucosyltransferase Enzymes that Produce Higher Molecular Weight Alpha-1,3-Glucan

As discussed above, SEQ ID NO:4 (GTF 6855) is an amino acid sequence of a glucosyltransferase that can be used to prepare amino acid substitutions. Briefly, certain amino acid substitutions can be made to SEQ ID NO:4 to provide a glucosyltransferase that produces alpha-1,3-glucan of higher molecular weight (as compared to alpha-1,3-glucan product of non-modified GTF 6855). These substitutions are listed in Tables E and F below. For collecting the data in these tables, each variant enzyme was entered into a glucan synthesis reaction with parameters as disclosed in U.S. Patent Appl. Publ. No. 2019/0078062 (corresponds to U.S. patent application Ser. No. 16/127,288), which is incorporated herein by reference. A glucosyltransferase with any of the amino acid substitution(s) listed in Tables E and F is contemplated to be useful in practicing the presently disclosed subject matter.

TABLE E
DPw of Insoluble Alpha-1,3-Glucan Produced by GTF 6855 (SEQ
ID NO: 4) and Single Amino Acid-Substituted Variants thereof

| GTF | DPw | GTF | DPw | GTF | DPw | GTF | DPw |
|---|---|---|---|---|---|---|---|
| 6855^{a} | 626 | T635H | 539 | P1499Y | 587 | A510E | 625 |
| V186A^{b} | 589 | T635W | 528 | Y219C | 591 | N904E | 554 |
| V186M | 580 | I636H | 521 | E243H | 631 | K930G | 637 |
| E194C | 580 | Y848E | 843 | A377I | 514 | K930V | 582 |
| L434N | 613 | D947G | 408 | I411F | 586 | D947F | 619 |
| A472C | 530 | F951Y | 325 | I411S | 591 | D947I | 610 |
| A472S | 374 | E849M | 610 | D425Q | 681 | D947K | 559 |
| A510E | 654 | Q1007A | 394 | L428V | 577 | D947N | 635 |
| A510I | 621 | D1003G | 486 | M529N | 560 | D947Q | 635 |
| A510V | 655 | A1022M | 303 | N531G | 977 | D947S | 603 |
| M529L | 558 | D1028L | 416 | G576R | 416 | D947V | 621 |
| R534G | 711 | D1028Q | 537 | Y580H | 554 | D947Y | 624 |
| R534I | 789 | A1057H | 624 | K593M | 792 | Q1007S | 578 |
| R534L | 763 | N1096A | 562 | I608Y | 708 | D1003N | 570 |
| R534M | 776 | Y1104M | 611 | N613G | 644 | I1026H | 621 |
| G576H | 436 | N1122K | 614 | N613L | 618 | D1028A | 568 |
| Q588L | 817 | E1132A | 589 | D617E | 419 | D1028M | 535 |
| I591K | 816 | E1132H | 611 | E621T | 603 | V1037A | 591 |
| I591R | 832 | E1132K | 610 | I627W | 506 | K1041A | 583 |
| Y605W | 524 | E1132R | 622 | S631D | 521 | K1041M | 648 |
| F607N | 561 | V1135K | 612 | S631E | 545 | D1080M | 554 |
| F607W | 624 | V1188E | 641 | S631R | 521 | F1244P | 589 |
| A610C | 799 | L1212N | 630 | G633W | 493 | F1244Q | 534 |
| N613I | 555 | E1250R | 606 | F634A | 523 | E1250H | 553 |
| N613M | 587 | T1381E | 612 | T635E | 561 | E1250K | 591 |
| N613T | 526 | T1431M | 625 | T635I | 648 | T1431Q | 663 |
| N613V | 578 | A1442R | 609 | T635Y | 518 | E1450D | 585 |
| K625A | 638 | E1450F | 611 | R722H | 793 | G1484P | 627 |
| K625M | 623 | E1450W | 618 | T728S | 769 | I1453G | 881 |
| A510E | 622 | I1453M | 635 | M732L | 791 | W1437N | 654 |
| S631T | 532 | V1491F | 604 | A777N | 755 | R722N | 766 |

^{a}GTF 6855, SEQ ID NO: 4. The DPw of insoluble alpha-1,3-glucan produced by GTF 6855 averaged to be about 626.
^{b}Each listed GTF with a substitution is a version of GTF 6855 comprising a substitution at a respective position, where the position number is in correspondence with the residue numbering of SEQ ID NO: 62.

TABLE F
DPw of Insoluble Alpha-1,3-Glucan Produced by GTF 6855 (SEQ
ID NO: 4) and Single Amino Acid-Substituted Variants thereof

| GTF | DPw |
|---|---|
| 6855^{a} | 558 |
| E567Q^{b} | 1001 |
| I591V | 859 |
| L661P | 842 |
| N743D | 700 |
| N743S | 937 |
| N743T | 874 |
| R741A | 831 |
| R741P | 871 |
| R741Q | 886 |
| R741S | 887 |
| R741T | 693 |
| T563A | 910 |
| V586T | 874 |

^{a}GTF 6855, SEQ ID NO: 4.
^{b}Each listed GTF with a substitution is a version of GTF 6855 comprising a substitution at a respective position, where the position number is in correspondence with the residue numbering of SEQ ID NO: 62.

### Example 1

### Glucosyltransferase-Catalyzed Synthesis of Alpha-Glucan Using Alpha-Glucan Ether as Primer

This Example describes using a glucosyltransferase to synthesize alpha-glucan using alpha-glucan ether as a primer. In particular, a glucosyltransferase enzyme that produces alpha-1,3-glucan was employed in a reaction comprising at least water, sucrose and carboxymethyl alpha-1,3-glucan (CMG) primer. The alpha-1,3-glucan material produced in this Example is contemplated to include a copolymer comprising a carboxymethylated alpha-1,3-glucan backbone and linear alpha-1,3-glucan side chain(s)/arm(s) (graft copolymer). It is also contemplated that copolymer was produced in which linear alpha-1,3-glucan was synthesized from the non-reducing end of the CMG primer; such a product is believed to include graft copolymer species, and/or species in which alpha-1,3-glucan arms were not synthesized off of the CMG primer. The copolymer products herein are collectively referred to as carboxymethyl glucan-primed glucan (CMGPG).

A series of eight reactions was performed; each reaction contained 50 or 100 g/L sucrose, 5 mM sodium phosphate buffer (pH 5.5), CMG primer (0, 0.5, 2.0, or 5.0 g/L), and 100 U/L of a glucosyltransferase (GTF). The CMG primer (termed herein as “CMG25”), which comprised carboxymethylated linear alpha-1,3-glucan (DPw of about 760), was aqueous-soluble and had degree of substitution of about 0.25. The GTF was an S. salivarius GTF modified in its catalytic domain such that the enzyme could produce more products (fructose and alpha-1,3-glucan with about 100% alpha-1,3 linkages), and less by-products (e.g., glucose, oligosaccharides such as leucrose and DP2-7 gluco-oligosaccharides), from sucrose substrate, as compared to the enzyme's unmodified counterpart. The General Methods section describes preparation of this non-native glucosyltransferase (Table A). Each reaction was prepared by charging a 250-mL disposable shake flask with 10 or 20 mL of a sterile-filtered 500-g/L sucrose stock solution; 5 mL of a 100-mM sodium phosphate buffer stock solution (pH 5.5); 0, 50, 200, or 500 mg of CMG25 dry powder; and a sufficient amount of water for a final volume of 100 mL. Solids were dissolved by vigorous shaking, after which GTF (0.226 mL, 100 U/L final concentration) was added to initiate each polymerization reaction; a 0.5-mL aliquot was taken from each solution just prior to GTF addition. All reactions were carried out in an incubator at 30° C. with shaking at 100 rpm. A 0.5-mL aliquot was withdrawn from each reaction 1 hour after start; a 0.1-mL aliquot was withdrawn at 2, 3 and 24 hours after reaction start. These aliquots were analyzed by HPLC. At 2 hours after reaction start, a 2.25-mL aliquot was withdrawn from both reactions containing 5 g/L CMG25 primer for rheological analysis. All aliquot samples upon their acquisition were placed in a heat block and heated to 80° C. for 10 minutes to stop the reactions.

After 24 hours, all the reactions were stopped by placing them in an 80° C. water bath for 30 minutes. The reactions containing 5 g/L CMG25 primer appeared as a hard semi-opaque gel. All the deactivated reactions were loaded onto 10-micron CHEMRUS disposable fritted filters and fully vacuum-filtered. The solids were then washed and filtered three times with 100 mL pure water to remove any water-soluble moieties, resulting in polymer wetcakes. Products of reactions having 5 g/L CMG25 primer were not homogenous and difficult to wash. Approximately 300-500 mg of each wetcake was set aside for SEC analysis, while the remaining material was dried in a 40° C., −45 cmHg vacuum oven for ˜3 days. Approximately 300 mg of each dry product was set aside for NMR analysis.

Wetcake samples were dissolved in 1% LiCl/DMSO and analyzed by SEC (General Methods) to determine apparent molecular weight and DPw of each polymer product. Dry polymer samples were dissolved in 3% LiCl/DMSO-d6. A small amount of D₂O was added. Each solution was then analyzed by ¹H NMR (General Methods) to determine the linkage profile of each of the polymer products and to calculate the mole percent (mol %) of CMG25 incorporation.

HPLC samples were prepared by diluting the 1-, 2- and 3-hour samples 10-fold with deionized, submicron-filtered water. The 0- and 24-hour samples were not diluted prior to preparation. All the samples were vortexed to ensure homogeneity, loaded onto a 0.45-micron Corning SPIN-X centrifuge filter, and centrifuged for 30 minutes at 12,000 rpm in a micro-centrifuge. The filtrate from each sample was loaded into a glass HPLC vial fitted with a low volume insert. The samples were analyzed using an Agilent 1200 series instrument equipped with an RI detector. Analysis was performed on two columns in parallel: BioRad AMINEX 87C (column 1) for separation of <DP3 sugars and BioRad AMINEX 42A (column 2) for separation of DP2-DP8+ oligosaccharides/polysaccharides. Linear calibration curves were constructed for sucrose (0.1-50 g/L), fructose (0.1-100 g/L), leucrose (0.1-100 g/L), and glucose (0.1-100 g/L) for quantitation on column 1. Linear calibration curves were constructed for DP2 (0.04-10 g/L), DP3 (0.04-10 g/L), DP4 (0.04-10 g/L), DP5 (0.04-10 g/L), DP6 (0.04-10 g/L), DP7 (0.04-10 g/L) and DP8+(0.25-10 g/L) for quantitation on column 2. Moieties with a DP greater than or equal to 8 (DP8+) were not well resolved on column 2. For quantification, a calibration curve was established using a lab-prepared dextran material (linear alpha-1,6 dextran with ˜20% alpha-1,2 branches, MW ˜17 kDa).

Table 2 (below) provides some results of the above analyses. CMG25 primer incorporation in the copolymer products was evidenced by NMR.

TABLE 2
Profiles of Glucosyltransferase Reactions (24-hour) Containing CMG25 Primer

| Start-of-Reaction | | Alpha-1,3- | Sucrose | | | |
|---|---|---|---|---|---|---|
| | CMG25 | Glucan | Consumption | Alpha-1,3-Glucan Product | | |
| Sucrose | Primer | Product Yield | Rate | | | Mol % CMG25 |
| (g/L) | (g/L) | (Glucosyl) | (g/L/h) | DPw | PDI | Incorporation |
| 50 | 0.0 | 81% | 5.7 | 1242 | 1.9 | 0.0% |
| | 0.5 | 82% | 6.8 | 1354 | 2.2 | 2.8% |
| | 2.0 | 85% | 6.8 | 1539 | 2.3 | 8.0% |
| | 5.0 | 82% | 7.3 | 1265 | 2.3 | 16.0% |
| 100 | 0.0 | 82% | 9.2 | 1159 | 1.8 | 0.0% |
| | 0.5 | 83% | 10.7 | 2115 | 2.8 | 1.6% |
| | 2.0 | 82% | 10.5 | 1285 | 2.6 | 4.0% |
| | 5.0 | 83% | 7.1 | 1506 | 2.6 | 12.0% |

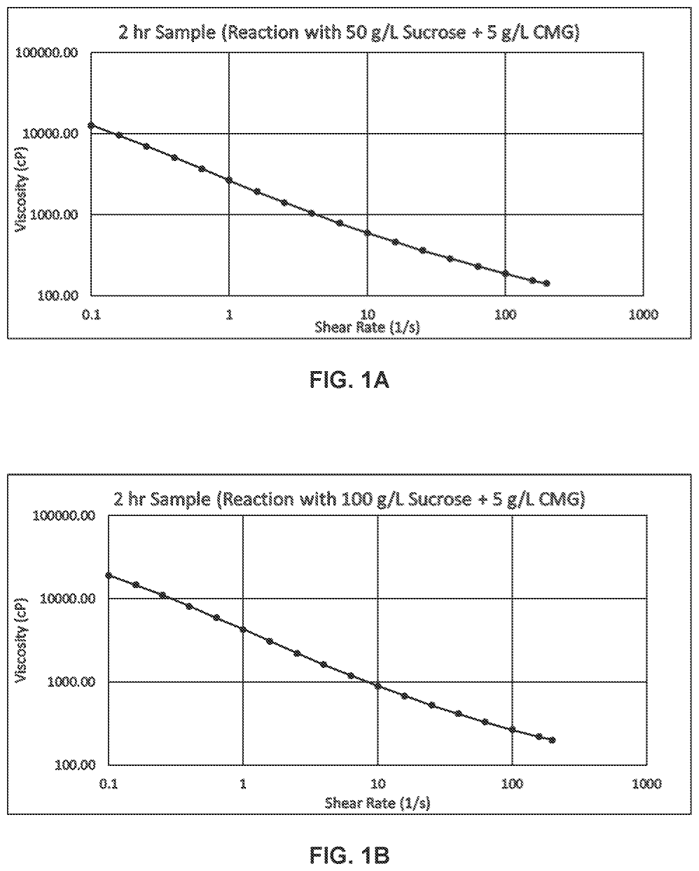

Shear viscosity of the 2-hour samples of reactions with 5 g/L CMG25 primer (50 and 100 g/L sucrose) were obtained using a Kinexus PRO+ rheometer fitted with cone and plate geometry. Results of this analysis are shown in FIGS. 1A and 1B. A dramatic increase in viscosity was observed for copolymer products of reactions with 5 g/L CMG25 primer at an early time point (2 hours), as confirmed by observing >10000 centipoise (cP) of shear viscosity at a 0.1/s shear rate. All shear viscosity measurements in this and the following Examples were made using the above equipment on homogenized samples of insoluble polymer products or samples of whole reactions. Viscosity results were obtained by sweeping from low to high (0.1/s→100/s) for one set of measurements, followed by sweeping from high to low (100/s→0.1/s); this procedure thus provided acquisition of replicate data. Since the values for each sweep in all cases herein were generally similar, only the high-to-low sweep viscosity measurements are shown in the figures (FIGS. 1-5) for clarity.

In this Example, a series of water-insoluble copolymers was produced in reactions comprising CMG25 primer and a glucosyltransferase that synthesizes alpha-1,3-glucan.

Based on observations made in the SEC analyses, these copolymers (CMGPG) are contemplated to comprise a carboxymethylated alpha-1,3-glucan backbone and linear alpha-1,3-glucan side chain(s)/arm(s) (graft copolymer). CMGPG may further comprise an alpha-1,3-glucan extension from the non-reducing end of the CMG25 primer; while most of these species are contemplated to have alpha-1,3-glucan arms, some species might not have these arms.

### Example 2

Glucosyltransferase-Catalyzed Synthesis of Alpha-Glucan Using Alpha-Glucan Ether as Primer (2-L Scale)

This Example describes a scale-up of select reactions described in Example 1, which were done in shake flasks (100-mL scale), to a 2-L scale in reactors. A similar set of conditions to that described in Example 1 was used to synthesize CMGPG.

Two reactions were prepared containing 22.5 or 100 g/L sucrose, 5 mM sodium phosphate buffer (pH 5.5), 10 g/L CMG25 primer and 100 U/L a GTF enzyme. The CMG primer and GTF enzyme were the same as those used in Example 1. Each reaction was prepared by dissolving 45 or 200 grams of sucrose and 20 grams of CMG25 in about 1 L of pure water using a magnetic stirrer. 10 mL of a 1 M sodium phosphate buffer stock solution (pH 5.5) was then added. The final volume was adjusted to 2 L using a volumetric flask. These solutions were each charged into a 2-L glass-jacketed reactor fitted with an overhead stirrer. A 0.5-mL aliquot was withdrawn from each solution at t=0 time point for HPLC analysis. An extra 5 mL was withdrawn at t=0 time point from the low sucrose (22.5 g/L) solution for rheological testing. Each reaction was then initiated by charging it with 4.535 mL of the GTF enzyme (100 U/L final concentration). The reactions were carried out at 30° C. Samples (1 mL) were withdrawn from each reaction to micro-centrifuge tubes at 1, 2, 3 and 24 hours following the start of the reactions. Each sample was heated to 80° C. for 10 minutes to deactivate the GTF enzyme. For HPLC, the samples were loaded onto 0.45-micron Corning SPIN-X centrifuge filters and centrifuged for 30 minutes at 12000 rpm on a table microcentrifuge. The filtrate from each sample was loaded into an HPLC glass vial fitted with a low volume insert.

Significant viscosity increases were observed for both reactions at early time points. In the 100 g/L sucrose reaction, viscosity broke between 2 and 3 hours after reaction initiation, with observable polymer precipitation followed by formation of a hard semi-opaque gel. This phenomenon caused the overhead stirring paddle to seize. Stirring was stopped for the remainder of the reaction. In the 22.5 g/L sucrose reaction, no break in viscosity was observed during this time period and through the end of 24 hours; the reaction mixture remained as an apparently homogenously-dispersed, high viscosity gel. Some increase in opacity was observed. At 24 hours reaction time, both reaction vessels were heated to 80° C. for 30 minutes to deactivate the GTF enzyme to stop the reactions.

Approximately 40 grams of reaction mixture were withdrawn from the completed, highly viscous, 22.5 g/L sucrose reaction for rheological characterization. Acetonitrile (2 L) was added to the remaining reaction mixture to precipitate the CMGPG product (to facilitate its isolation for analysis, given the high viscosity), stirred for 24 hours, and allowed to settle for 48 hour before separating the solids by centrifugation at 5000 rpm at −20° C. in 1-L centrifuge bottles. After decanting the supernatant, which contained soluble sugars and oligosaccharides, CMGPG (appeared as a gel layer) was collected from the bottom of the centrifuge tubes. This gel was placed in a vacuum oven at 40° C., −45 cmHg for about 3 days, until dry.

HPLC, NMR and SEC analyses were performed as described in Example 1. Table 3 (below) provides some results of these analyses.

TABLE 3
Profiles of Glucosyltransferase Reactions
(24-hour) Containing 10 g/L CMG25 Primer

| Start-of- | Alpha-1,3- | Sucrose | | | |
|---|---|---|---|---|---|
| Reaction | Glucan | Consumption | Alpha-1,3-Glucan Product | | |
| Sucrose | Product Yield | Rate | | | Mol % CMG25 |
| (g/L) | (Glucosyl) | (g/L/h) | DPw | PDI | Incorporation |
| 22.5 | 63% | 4.1 | 1545 | 3.0 | 52.0% |
| 100 | 84% | 8.2 | 1628 | 3.1 | 16.0% |

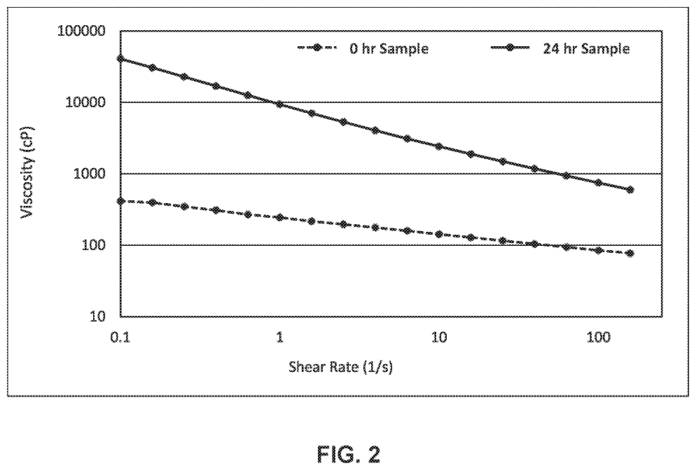

Rheology tests were performed on the 0- and 24-hour samples of the low sucrose (22.5 g/L) reactions (these samples are whole samples of each reaction; i.e., insoluble glucan product was not purified and homogenized before viscosity testing). Shear viscosity from 0.1 to 100 s⁻¹ was determined using the method described in Example 1; results are shown in FIG. 2. The 24-hour sample had shear viscosity about 100 times higher than that of the 0-hour sample at shear rate of 0.1/s. The final concentration of grafted polymer was approximated to be 1.6% in the 2-L reaction (22.5 g/L sucrose) based on a recovery of about 32 grams of dry, pure material from the reaction at 24 hours.

### Example 3

### Dispersion and Viscosity Analysis of Alpha-Glucan Ether-Primed Alpha-Glucan

This Example describes, among other things, the recovery of high viscosity conditions upon dispersion/re-suspension of the CMGPG that was isolated and dried from the 22.5 g/L sucrose reaction described in Example 2 (i.e., the high viscosity observed in the terminated reaction, which contained “never-dried” CMGPG product, could be achieved by dispersing the CMGPG following its isolation from the reaction mixture and being dried). This represents an advantage over alpha-1,3-glucan that is synthesized without an ether primer; such non-ether-primed alpha-1,3-glucan not only has lower viscosity compared to CMGPG (in their respective never-dried states such as in reaction mixtures), but also is more difficult to disperse after drying (thereby not achieving the viscosity level of its never-dried forerunner). Also, this Example describes a lack of viscosity increase when mixing non-primed alpha-1,3-glucan reaction mixtures with CMG primer, which indicates that the enhanced viscosity observed with CMGPG is likely due to CMGPG itself.

Two GTF reactions were performed to generate mixtures containing non-primed alpha-1,3-glucan (i.e., glucan was synthesized in reactions that did not contain primer). Similar conditions were used as described in Example 2, except that CMG primer was omitted. Briefly, two 250-mL disposable shake flasks were charged with 1 or 2.25 g of sucrose (10 or 22.5 g/L final concentration), 5 mL of 100 mM sodium phosphate buffer stock (pH 5.5), and a sufficient volume of deionized, submicron-filtered water to provide a final volume of 100 mL. A 0.5-mL aliquot was taken from each solution before 0.226 mL of GTF enzyme (100 U/L final concentration, same enzyme as used in Example 2) was added to initiate each polymerization reaction. Each reaction was carried out in an incubator shaker at 30° C. with shaking at 100 rpm. After 24 hours, a 1-mL sample was withdrawn from each reaction and heated to 80° C. for 10 minutes to stop the reaction. Also at 24 hours, the entire reactions were terminated by placing the reaction vessels into an 80° C. water bath. The terminated reactions were considered to be alpha-1,3-glucan reaction mixtures, given the presence of the aqueous-insoluble glucan product.

The 0.5-mL and 1-mL samples were centrifuged at 12000 rpm for 10 minutes on a microcentrifuge to remove insoluble material. Superatants were filtered through 0.45-micron PTFE Whatman® syringeless filters and then analyzed by HPLC as described in Example 1.

Several preparations were made, as described in Tables 4 and 5 below, to test the effects of mixing the alpha-1,3-glucan reaction mixtures prepared above with CMG primer (same as used in Example 1), as well as to test the ability of isolated CMGPG (as produced in the 22.5 g/L sucrose reaction of Example 2) to regain its reaction mixture viscosity after resuspension/dispersion in water.

TABLE 4
Preparation Components

| | Batch | Alpha-1,3-Glucan |
|---|---|---|
| Starting Material | ID | (w/v %) |
| Alpha-1,3-glucan reaction mixture | −01 | 0.38% |
| (non-primed, 10 g/L sucrose reaction, | | |
| terminated, Example 3) | | |
| Alpha-1,3-glucan reaction mixture | −02 | 0.75% |
| (non-primed, 22.5 g/L sucrose reaction, | | |
| terminated, Example 3) | | |
| CMG dry powder | −303 | dry powder |
| (DoS = 0.25, DPw 760; CMG25) | | |
| CMGPG dry powder | | dry powder |
| (from 22.5 g/L sucrose reaction as described in | | |
| Example 2) | | |

TABLE 5
Aqueous Preparations of Alpha-1,3-Glucan (Non-Primed), CMG, and/or CMGPG

| | | Medium | Final | Total of Alpha-1,3-Glucan, |
|---|---|---|---|---|
| Sample | CMG or CMGPG | (Glucan Reaction | Volume | CMG, and/or CMGPG |
| ID | (w/v %) | Mixture or H₂O) | (mL) | (w/v %) |
| 1 | 0.0% | −01^{b} | 50 | 0.38% |
| 2 | 0.0% | −02^{b} | 50 | 0.75% |
| 3 | CMG, 1% batch −303^{a} | −01^{b} | 50 | 1.39% |
| 4 | CMG, 1% batch −303^{a} | −02^{b} | 50 | 1.75% |
| 5 | CMG, 1% batch −303^{a} | H₂O | 50 | 1.00% |
| 6 | CMG, 1.7% batch −303^{a} | H₂O | 50 | 1.72% |
| 7 | CMGPG^{a}, 1% | H₂O | 50 | 1.02% |
| 8 | CMGPG^{a}, 0.5% | H₂O | 50 | 0.50% |

^{a}As listed in Table 4.
^{b}As listed in Table 4 under Batch ID.

All the preparations of Table 5 (Sample 1-8) were homogenized at about 13500 rpm for at least 2 minutes, or until appearance was homogenous. Upon the initial addition of water to the dry CMGPG samples, a low viscosity, non-homogenous slurry was formed; the dry particles appeared to swell, but did not disperse. Upon homogenization (13500 rpm) of these CMGPG slurries, viscosity rapidly built up, forming an apparently homogenous dispersion resembling the reaction mixture from which the CMGPG was originally isolated.

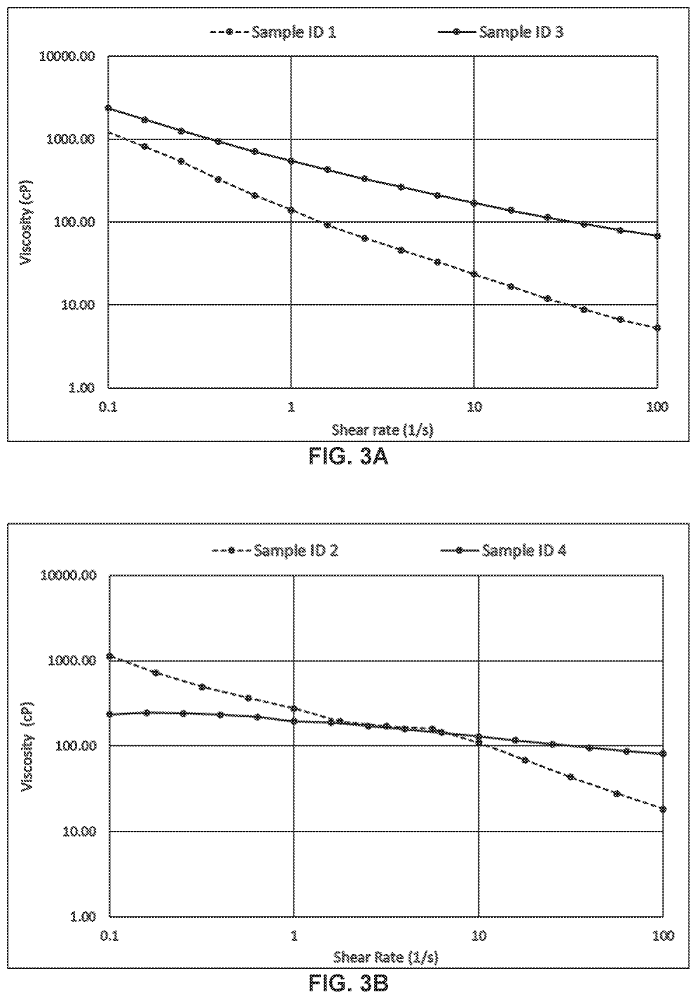

Shear viscosity measurements of all the homogenized samples (above) were obtained as described in Example 1, and are shown in FIGS. 3A-D. Samples 2-8 were analyzed using plate/plate geometry, while Sample 1 was analyzed using cup/vane geometry. FIG. 3A compares the viscosity profile of the end-of-reaction mixture from non-primed 10 g/L sucrose reaction (Sample 1) with the viscosity profile of the same end-of-reaction mixture as further mixed with 1 w/v % CMG25 primer (Sample 3). Sample 3 represents a hypothetical end-of-reaction mixture in which a GTF reaction with 10 g/L sucrose and 1 w/v % CMG25 primer was conducted, but no priming occurred. FIG. 3B compares the viscosity profile of the end-of-reaction mixture from non-primed 22.5 g/L sucrose reaction (Sample 2) with the viscosity profile of the same end-of-reaction mixture as further mixed with 1 w/v % CMG25 primer (Sample 4). Sample 4 represents a hypothetical end-of-reaction mixture in which a GTF reaction with 22.5 g/L sucrose and 1 w/v % CMG25 primer was conducted, but no priming occurred. The data in FIGS. 3A-B indicate that addition of 1 w/v % CMG25 to terminated, non-primed alpha-1,3-glucan reaction mixtures only mildly changes shear viscosity profile. If, for sake of argument, priming did not occur in Example 2, the end-of-reaction mixture of the 22.5 g/L sucrose+CMG reaction in Example 2 would have exhibited a viscosity profile similar to the profile of Sample 3 in FIG. 3B, but that was not the case. Indeed, the actual viscosity profile of that reaction (FIG. 2, 24-hr) was significantly higher than the profile of Sample 4 (FIG. 3B). For instance, as a simple but direct comparison, the viscosity at a shear rate of 0.1 s⁻¹ was 235 cP for Sample 4 (FIG. 3B), but was 40720 cP for the primed end-of-reaction mixture in Example 2 (FIG. 2). FIG. 3C shows the viscosity profiles of CMGPG preparations at 0.5 w/v % (Sample 8) and 1 w/v % (Sample 7), respectively. The CMGPG in these samples was the graft copolymer product of the 22.5 g/L sucrose+CMG priming reaction of Example 2. The data indicate that CMGPG was able to dramatically increase viscosity even at 0.5 w/v % and 1 w/v % concentrations. Therefore, CMGPG was confirmed to be the highly viscous material in the end-of-reaction mixture of Example 2. A 1 w/v % CMGPG preparation had a similar or comparable viscosity profile (Sample 7, FIG. 3D) as the profile of the end-of-reaction mixture in Example 2 (FIG. 2) in which the CMGPG content was estimated to be ˜1.6 w/v %. This observation suggests that CMGPG powder could be dispersed to regain the viscosity that it exhibited when prepared in its original reaction-mixture state (“never-dried” state). FIG. 3D compares the viscosity profile of 1 w/v % CMGPG (Sample 7) with CMG25 primer at 1 w/v % (Sample 5) and 1.7 w/v % (Sample 6). The 1 w/v % CMGPG preparation (Sample 7) was much more viscous than CMG25 at the same 1 w/v % concentration (Sample 5). The 1.7 w/v % CMG25 preparation of Sample 6 represents a reaction in which no priming occurred; for Sample 6, the non-primed alpha-1,3-glucan portion of the end-of-reaction mixture of Example 2 was replaced by CMG25 primer. In this test, the addition of about 0.7 w/v % of CMG25 to the starting concentration of 1 w/v % was not able to boost the viscosity level to that of the 1 w/v % CMGPG dispersion. Since the final CMGPG concentration of the actual end-of-reaction mixture of Example 2 was estimated to be ˜1.6 w/v %, which was very similar to the 1.7 w/v % CMGPG concentration in Sample 6, these two tests can be used for a direct comparison on viscosity. The viscosity at 0.1 s⁻¹ shear rate was 40720 cP for the end-of-reaction mixture of Example 2 (FIG. 2), but only 591 cP for the 1.7 w/v % CMG25 (Sample 6, FIG. 3D). This observation again highlights the high viscosity nature of CMGPG as compared with that of CMG25 at a higher concentration of ˜1.6 w/v %.

To summarize this Example, the priming reactions of Example 2 were further confirmed by the viscosity changes observed among samples representing different reaction conditions, either real or hypothetical. Isolated and dried CMGPG powder was demonstrated to be highly viscous when dispersed into an aqueous solution, even at the low concentrations of 0.5 w/v % and 1 w/v %. CMGPG preparations were significantly more viscous than solutions of CMG25 primer at the same/similar concentrations. CMGPG was confirmed to be the main cause of the high viscosity of the end-of-reaction mixtures of Example 2.

### Example 4

### Effects of Increased Alpha-Glucan Ether DoS (˜0.31) on Priming of Alpha-Glucan Synthesis in Glucosyltransferase Reactions

This Example, coupled with Examples 1 and 2, describes how alpha-1,3-glucan synthesis reactions that contain alpha-glucan primer are affected by the DoS of the primer. Herein, a soluble carboxymethylated linear alpha-1,3-glucan was used as a primer (˜760 DPw, ˜0.31 DoS, termed “CMG31” herein) for the enzymatic synthesis of insoluble alpha-1,3-glucan. The CMG31 primer only differs from the primer used in the above Examples (CMG25) by its higher DoS of ˜0.31. Alpha-1,3-glucan products of this Example likely represent additional forms of CMGPG as described in the above Examples (i.e., graft copolymer with a carboxymethylated alpha-1,3-glucan backbone and alpha-1,3-glucan chain(s)/arm(s)). Similar to the observations in the above Examples, the graft copolymer products of this Example were observed to dramatically increase reaction viscosity.

A series of three reactions was performed with about 10, 20, or 100 g/L sucrose, 5 mM sodium phosphate buffer (pH 5.5), 10 g/L of CMG31 primer (see above) and 100 U/L of a GTF enzyme. The GTF enzyme was the same as that used in Example 1. Each reaction mixture was prepared by charging a 250-mL disposable shake flask with 2, 4, or 20 ml of a sterile-filtered 500 g/L sucrose stock solution, 5 mL of a 100 mM sodium phosphate buffer stock solution (pH 5.5), 1 g of CMG31 dry powder and a sufficient volume of deionized, submicron-filtered water to provide a final volume of 100 mL. The solids were dissolved with vigorous shaking, after which a 0.5-mL aliquot was taken from each solution (t=0 time point) and 0.226 mL of the GTF enzyme (100 U/L final concentration) was added to initiate polymerization. All reactions were carried out in an incubator at 30° C. with shaking at 100 rpm. A 1-mL aliquot was withdrawn from each reaction at 1, 2, 3, and 24 hours after reaction initiation for HPLC analysis. All aliquot samples were placed in a heat block at 80° C. for 10 minutes to deactivate the GTF.

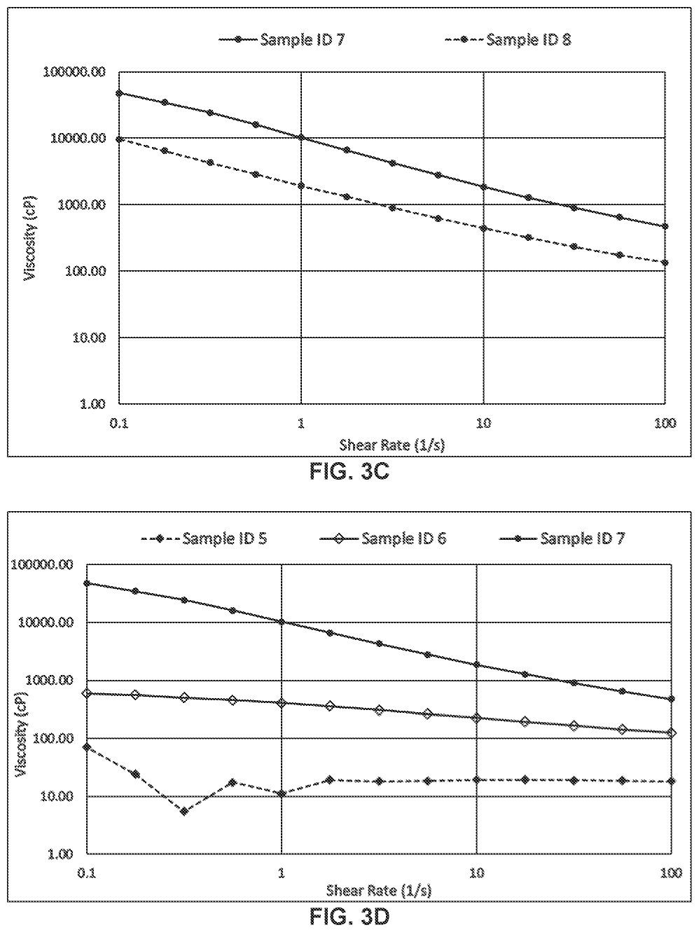

After 24 hours, all reaction vessels were placed in an 80° C. water bath for 30 minutes to stop the reactions. The reaction with 100 g/L sucrose appeared as a hard semi-opaque gel, while the other two reactions appeared to be viscous dispersions (thus, all three reactions could be characterized as reaction mixtures). The 100 g/L sucrose reaction was processed as described in Example 1 to generate dry polymer. Each of the deactivated 10 g/L and 20 g/L sucrose reactions had about 5 mL withdrawn for rheology analysis and about 1 mL withdrawn for SEC analysis, before being dried in a 40° C., −45 cmHg vacuum oven for about 3 days. HPLC, SEC, NMR and rheological analyses were performed largely as described in Example 1, except rheology was measured with cone/plate geometry. Table 6 (below) and FIG. 4 provide some results of these analyses.

TABLE 6
Profiles of Glucosyltransferase Reactions
(24-hour) Containing 10 g/L CMG31 Primer

| Start-of- | Alpha-1,3- | Sucrose | | | |
|---|---|---|---|---|---|
| Reaction | Glucan | Consumption | Alpha-1,3-Glucan Product | | |
| Sucrose | Product Yield | Rate | | | Mol % CMG31 |
| (g/L) | (Glucosyl) | (g/L/h) | DPw | PDI | Incorporation |
| 10.2 | 45% | 1.20 | 1942 | 1.556 | 70.97% |
| 21.6 | 64% | 1.89 | 1327 | 1.866 | 54.84% |
| 107.3 | 77% | 3.79 | 1147 | 1.694 | 22.58% |

The reactions appeared, for the most part, to proceed in a manner similar to that of the reactions described in Examples 1 and 2. For example, high viscosities were observed for samples of low sucrose reactions (10 g/L and 20 g/L sucrose) at 24 hours; this observation was confirmed by rheology data (FIG. 4). This observation, along with NMR data showing CMG31 primer incorporation into the insoluble products, for example, support the conclusion that the glucosyltransferase reactions in this Example produced the graft copolymer, CMGPG. However, compared to the glucosyltransferase reactions of Examples 1 and 2, which employed a CMG primer with a DoS of about 0.25, use of the higher DoS primer, CMG31 (DOS˜0.31), seemed to reduce the sucrose consumption rate of the glucosyltransferase reactions in this Example.

### Example 5

### Effects of Increased Alpha-Glucan Ether DoS (˜0.70) on Priming of Alpha-Glucan Synthesis in Glucosyltransferase Reactions

This Example, coupled with Examples 1, 2 and 4, describes how alpha-1,3-glucan synthesis reactions that contain alpha-glucan primer are affected by the DoS of the primer. Herein, soluble carboxymethylated linear alpha-1,3-glucan was used as a primer (˜1220 DPw, ˜0.70 DoS, termed “CMG70” herein) for the enzymatic synthesis of insoluble alpha-1,3-glucan. The CMG70 primer differs from the primers used in the above Examples (CMG25 and CMG31) by its higher DoS of ˜0.70 and DPw of 1220. Alpha-1,3-glucan products of this Example likely represent additional forms of CMGPG as described in the above Examples (i.e., graft copolymer with a carboxymethylated alpha-1,3-glucan backbone and alpha-1,3-glucan chain(s)/arm(s)). Similar to the observations in the above Examples, the graft copolymer products of this Example were observed to dramatically increase reaction viscosity.

A series of three reactions was performed with about 10, 20, or 100 g/L sucrose, 5 mM sodium phosphate buffer (pH 5.5), 10 g/L of CMG70 primer (see above) and 100 U/L of a GTF enzyme. The GTF enzyme was the same as that used in Example 1. Each reaction mixture was prepared by charging a 250-mL disposable shake flask with 2, 4, or 20 mL of a sterile-filtered 500 g/L sucrose stock solution, 5 mL of a 100 mM sodium phosphate buffer stock solution (pH 5.5), 1 g of CMG70 dry powder and a sufficient volume of deionized, submicron-filtered water to provide a final volume of 100 mL. The solids were dissolved with vigorous shaking, after which a 0.5-mL aliquot was taken from each solution (t=0 time point) and 0.226 mL of the GTF enzyme (100 U/L final concentration) was added to initiate polymerization. All reactions were carried out in an incubator at 30° C. with shaking at 100 rpm. A 1-mL aliquot was withdrawn from each reaction at 1, 2, 3, and 24 hours after reaction initiation for HPLC analysis. All aliquot samples were placed in a heat block at 80° C. for 10 minutes to deactivate the GTF.

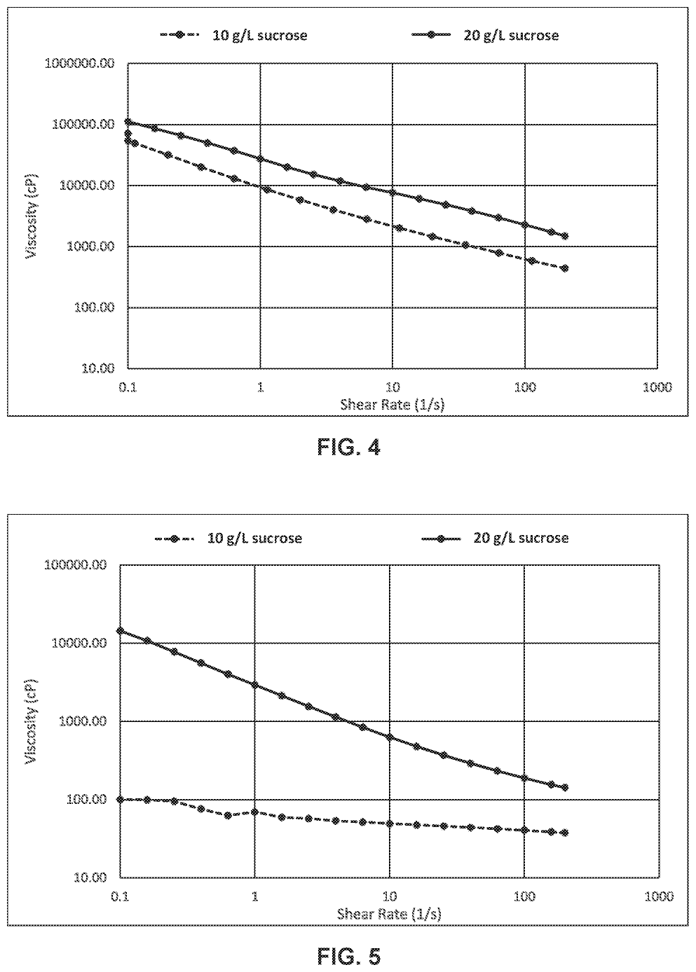

After 24 hours, all reaction vessels were placed in an 80° C. water bath for 30 minutes to stop the reactions. The reaction with 100 g/L sucrose appeared as a hard semi-opaque gel, while the reaction with 20 g/L sucrose appeared as a viscous opaque dispersion. The reaction with 10 g/L sucrose appeared as an opaque dispersion with relatively unchanged viscosity relative to the starting point. The deactivated 100 g/L sucrose reaction was processed as described in Example 1 to generate dry polymer. The deactivated 10 g/L and 20 g/L sucrose reactions had about 5 mL withdrawn for rheology analysis and about 1 mL withdrawn for SEC analysis, before being dried in their entirety in a 40° C., −45 cmHg vacuum oven for about 3 days. HPLC, SEC, NMR and rheological analyses were performed largely as described in Example 1, except rheology was measured with cone/plate geometry. Table 7 (below) and FIG. 5 provide some results of these analyses.

TABLE 7
Profiles of Glucosyltransferase Reactions
(24-hour) Containing 10 q/L CMG70 Primer

| Start-of- | End-of- | Alpha-1,3- | Sucrose | | | |
|---|---|---|---|---|---|---|
| Reaction | Reaction | Glucan | Consumption | Alpha-1,3-Glucan Product | | |
| Sucrose | Sucrose | Product Yield | Rate | | | Mol % CMG70 |
| (g/L)) | (g/L) | (Glucosyl) | (g/L/h) | DPw | PDI | Incorporation |
| 11 | 5 | 18% | 0.61 | 1950.4 | 1.701 | 61.43% |
| 22 | 9 | 41% | 0.75 | 1290.3 | 1.650 | 41.43% |
| 113 | 20 | 69% | 3.49 | 1109.6 | 1.532 | 15.71% |

All the reactions were found not to go to completion (Table 7, note end-of-reaction sucrose levels), and proceeded at a significantly reduced sucrose consumption rate relative to what was described in Examples 1, 2 and 4. These results might indicate some level of inhibition of glucosyltransferase enzymatic activity as primer DoS is increased. However, all the reactions still were able to produce at least some amount of CMGPG (also note the results in Example 7 below regarding use of primer CMG110). That the ˜10 g/L sucrose reaction did not produce a high viscosity reaction mixture (FIG. 5) probably was due to the low yield of product in this reaction (Table 7).

### Example 6

### Glucosyltransferase-Catalyzed Synthesis of Alpha-Glucan Using Alpha-Glucan Ether as Primer and Zeta Potential Analysis of the Alpha-Glucan Product

This Example describes glucosyltransferase-catalyzed synthesis of alpha-1,3-glucan graft copolymer using CMG31 (above) as a primer (i.e., CMGPG was produced). The graft copolymer products of these reactions, CMGPG, were then subject to zeta potential and particle size analyses, showing that CMGPG has significantly altered structural features as compared to non-primed alpha-1,3-glucan.

A series of three reactions was performed with 108 g/L sucrose, 2 mM sodium phosphate buffer (pH 5.8), 250 U/L of a GTF, and CMG31 primer (i.e., CMG with ˜760 DPw and ˜0.31 DoS). The GTF was an S. salivarius GTF modified in its catalytic domain such that the enzyme could produce more products (fructose and alpha-1,3-glucan with about 100%alpha-1,3 linkages), and less by-products (e.g., glucose, oligosaccharides such as leucrose and DP2-7 gluco-oligosaccharides), from sucrose substrate, as compared to the enzyme's unmodified counterpart. The General Methods section describes preparation of this non-native glucosyltransferase (Table B). CMG31 primer was added in 0%, 1% and 5% concentrations in relation to the 45 g/L alpha-1,3-glucan that was expected to be produced by each reaction. Each reaction was prepared with a master stock solution that was made by dissolving 324 g sucrose and 0.82 g anhydrous sodium phosphate in deionized water to 3 L using an overhead mixer. The pH of the master stock solution was adjusted to 5.8 by addition of 0.1 N sodium hydroxide solution. 1-L aliquots were sterile-filtered from the master stock solution and charged into three 1000-mL jacketed glass reactors equipped with a PBT impeller and overhead mixer. Dry CMG31 powder was added to each reactor in the amounts of 0, 45 and 225 mg and dissolved in the media. All the reactors were connected to a recirculating water bath, which maintained the reaction temperature at 38.5° C. with agitation. A 5.0-mL aliquot was taken from each solution just before addition of 1.80 mL GTF enzyme (250 U/L final concentration) to initiate the polymerization reactions. A 5.0-mL aliquot was withdrawn at 1, 2, 3, 3.5, 4, 5 and 24 hours after starting each reaction. All these samples were placed in a heat block and heated to 80° C. for 10 minutes to deactivate the enzyme, and then analyzed by HPLC for sugars and soluble oligomers.

After 24 hours, all reaction vessels were heated to 75° C. for 30 minutes to stop the reactions, by adjusting the temperature of the recirculating water baths. All the deactivated reactions were then vacuum-filtered with 2-L Büchner funnels and Whatman® Grade 541 filter paper. The filtered insoluble products were then washed and filtered with 4 L deionized water to remove any water-soluble moieties, thereby providing polymer wetcakes. Approximately 5-10 g of each wetcake was set aside for SEC analysis, about 1-2 g of each wetcake was set aside for particle size distribution (PSD) analysis, and about 15-28 g of the wetcake material was dried in an 85° C., −45 cmHg vacuum oven for about 24 hours. Wet cake was also set aside for zeta potential analysis.

Wetcakes were analyzed by SEC to determine apparent molecular weight and DPw of the final products. HPLC samples were vortexed to ensure homogeneity, loaded onto a Corning SPIN-X UF6 centrifuge filter, and centrifuged for 10 minutes at 4400 rpm. The filtrate from each sample was loaded into a glass HPLC vial and analyzed using an Agilent 1200 series instrument equipped with an RI detector. Analysis was performed on two columns in parallel: BioRad AMINEX 87C (column 1) for separation of <DP3 sugars and BioRad AMINEX 42A (column 2) for separation of DP2-DP8+ oligo/polysaccharides. Linear calibration curves were constructed for sucrose (0.1-50 g/L), fructose (0.1-100 g/L), leucrose (0.1-100 g/L), and glucose (0.1-100 g/L) for quantitation on column 1. Linear calibration curves were constructed for DP2 (0.04-10 g/L), DP3 (0.04-10 g/L), DP4 (0.04-10 g/L), DP5 (0.04-10 g/L), DP6 (0.04-10 g/L), DP7 (0.04-10 g/L) and DP8+(0.25-10 g/L) for quantitation on column 2. Moieties with a DP greater than or equal to 8 (DP8+) were not well resolved on column 2. For quantification, a calibration curve was established using a lab-prepared dextran material (linear alpha-1,6 dextran with ˜20% alpha-1,2 branches, MW ˜17 kDa).

Particle size distribution (PSD) was measured on a Malvern MASTERSIZER 2000 laser diffraction device (Malvern Instruments, Westborough, MA), accordingly (e.g., see ISO 13320-1:1999, Particle size analysis—Laser diffraction methods—Part 1: General principles; incorporated herein by reference). Particulars of the PSD measurement procedure were as follows:

### Instrument Settings

Particle Refractive Index: 0 (Fraunhofer optical model).
Dispersant: clean deionized water (typically pH 5.5, in some cases adjusted to pH 3.7).
Dispersant Refractive Index: 1.33.
Signal averaging: 15000 snaps per measurement (15 sec).
Dispersion Unit: Malvern HYDRO S general-purpose automated sample dispersion unit.
Pump/Stirrer Speed: 2000 rpm.

### Procedure

1. Fill sample reservoir with clean water.
2. Start measurement: measure the MASTERSIZER's background signals.
3. Gently shake vial containing sonicated or non-sonicated sample of resuspended wet cake by hand for 5 seconds to mix well and re-suspend samples.
4. Add sample dropwise to HYDRO S sample reservoir until 5-10% obscuration is obtained.
5. Prompt unit to continue with diffraction measurement. PSD is automatically calculated by MASTERSIZER and data is stored for analysis.
   PSD analysis was performed with or without sonication (50 W horn) for three minutes prior to analysis with the laser diffraction device.

Zeta potential was measured with a ZETASIZER NANO ZS instrument (Malvern, Westborough, MA). For each product, a wet cake sample was used to prepare a dilute, see-through dispersion, which was then adjusted to pH 8 with KOH. This preparation was then sonicated for one minute using a Cole Parmer GEX 750 Ultrasonic Processor (750 Watts, 20 KHz, Amp 49%). Each sample was then placed in a cuvette having a pair of immersed platinum electrodes. An alternating electric potential was applied across the electrodes, and the motion of the particles was observed using a back-scattered laser light and an auto-correlation technique similar to dynamic light-scattering. The electrophoretic mobility of particles was measured by phase-sensitive detection, and this mobility was used to determine the zeta potential. Three consecutive 10 to 15-minute cycles of measurement were taken for each sample; these measurements were averaged together.

Tables 8 and 9 (below) provide some results of the above analyses.

TABLE 8
Profiles of Glucosyltransferase Reactions
(24-hour) Containing CMG31 Primer

| | | Alpha-1,3- | | | | |
|---|---|---|---|---|---|---|
| Start-of-Reaction | | Glucan | Sucrose | Alpha-1,3-Glucan Product | | |
| | CMG31 | Product | Consumption | | | Zeta |
| Sucrose | Primer | Yield | Rate | | | Potential |
| (g/L) | Conc.^{a} | (Glucosyl) | (g/L/h) | DPw | PDI | (mV) |
| 108 | 0% | 92.6% | 12.2 | 905 | 6.51 | −3.25 |
| | 1% | 92.5% | 10.3 | 896 | 5.07 | −17.39 |
| | 5% | 94.3% | 8.5 | 915 | 2.54 | −40.10 |

^{a}Percent based on ratio of CMG31 primer mass to mass of expected alpha-1,3-glucan product (based on expected 45 g/L production by reaction).

TABLE 9
Particle Size Distribution (PSP) of Insoluble Alpha-1,3-Glucan
Products of Glucosyltransferase Reactions (24-hour)

| | Alpha-1,3-Glucan Product | | | | | |
|---|---|---|---|---|---|---|
| CMG31 | Unsonicated | | | Sonicated (3 min w/horn) | | |
| Primer | D10 | D50 | D90 | D10 | D50 | D90 |
| Conc.^{a} | (μm) | (μm) | (μm) | (μm) | (μm) | (μm) |
| 0% | 22.269 | 64.368 | 170.338 | 2.474 | 4.638 | 8.807 |
| 1% | 46.976 | 90.793 | 150.633 | 0.082 | 0.270 | 2.372 |
| 5% | 13.723 | 45.214 | 218.567 | 0.074 | 0.179 | 4.553 |

^{a}Percent based on ratio of CMG31 primer mass to mass of expected alpha-1,3-glucan product (based on expected 45 g/L production by reaction).

As shown in Table 8, there was a dramatic difference in the zeta potential of non-primed alpha-1,3-glucan compared to the zeta potentials of alpha-1,3-glucan synthesized off CMG primer (i.e., CMGPG). The CMGPG product zeta potential was generally proportional to the concentration of CMG primer used in the reaction. Since the zeta potential of the dispersed CMGPG was greater (more than ±15 mV) compared to the zeta potential of dispersed non-CMG-primed product, dispersions of CMGPG should be more stable (e.g., less prone to aggregation over time). In addition, as shown in Table 9, the PSD of insoluble products was affected by the incorporation of CMG into the alpha-1,3-glucan polymer. This incorporation allowed for a reduction in CMGPG particle size to less than one micron (compared to non-primed polymer) as observed when imparting mild energy to the system in the form of sonication. These PSD data likely reflect, at least in part, the greater zeta potential of CMGPG.

### Example 7

### Glucosyltransferase-Catalyzed Synthesis of Alpha-Glucan Using Alpha-Glucan Ether as Primer and Structural Analysis of the Alpha-Glucan Product

This Example describes glucosyltransferase-catalyzed synthesis of alpha-1,3-glucan graft copolymer using CMG31 (above) or CMG of ˜1.1 DoS and ˜1220 DPw (“CMG110” herein) as a primer (i.e., CMGPG was produced). The graft copolymer products of these reactions were then subject to zeta potential analysis, showing that CMGPG has significantly altered structural features as compared to non-primed alpha-1,3-glucan.

A series of ten reactions was performed with 108 g/L sucrose, 2 mM sodium phosphate buffer (pH 5.8), 250 U/L of a GTF, and a CMG primer (CMG31 or CMG110). The GTF enzyme was the same as that used in Example 6. CMG primer was added in 0%, 1% and 5% concentrations in relation to the 45 g/L alpha-1,3-glucan that was expected to be produced by each reaction at 38.5° C. and 28.0° C. Each reaction was prepared with a master stock solution that was made by dissolving 1040 g sucrose and 2.72 g anhydrous sodium phosphate in deionized water to 10 L using an overhead mixer. The pH of the master stock solution was adjusted to 5.8 by addition of 0.1 N sodium hydroxide solution. 1-L aliquots were sterile-filtered from the master stock solution and charged into ten 1000-mL jacketed glass reactors equipped with a PBT impeller and overhead mixer. Dry CMG powder was added to each reactor in the amounts of 0, 45 and 225 mg and dissolved in the media. All the reactors were connected to a recirculating water bath, which maintained the reaction temperature at 38.5° C. or 28.0° C. with agitation. A 5.0-mL aliquot was taken from each solution just before addition of 1.80 mL GTF enzyme (250 U/L final concentration) to initiate the polymerization reactions. A 5.0-mL aliquot was withdrawn at 1, 2, 3, 3.5, 4, 5 and 24 hours after starting each reaction. All these samples were placed in a heat block and heated to 80° C. for 10 minutes to deactivate the enzyme, and then analyzed by HPLC for sugars and soluble oligomers.

After 24 hours, all reaction vessels were heated to 75° C. for 30 minutes to stop the reactions, by adjusting the temperature of the recirculating water baths. Preparation for, and performance of, HPLC, SEC, PSD and zeta potential analyses were as described in Example 6.

Table 10 (below) provides some results of the above analyses.

TABLE 10
Profiles of Glucosyltransferase Reactions
(24-hour) Containing CMG31 or CMG110 Primer

| | | | Sucrose | Alpha-1,3-Glucan Product | | |
|---|---|---|---|---|---|---|
| Reaction Conditions | | | Consumption | | | Zeta |
| | Primer | Temp. | Rate | | | Potential |
| Primer | Conc.^{a} | (° C.) | (g/L/h) | DPw | PDI | (mV) |
| — | 0% | 28.0 | 6.4 | 1719 | 4.82 | −0.66 |
| CMG31 | 1% | 28.0 | 16.6 | 1623 | 2.76 | −1.42 |
| CMG31 | 5% | 38.5 | 15.3 | 927 | 0.37 | −35.33 |
| CMG110 | 1% | 28.0 | 17.9 | 2043 | 2.23 | −27.50 |
| CMG31 | 5% | 28.0 | 19.3 | 1337 | 2.34 | −33.77 |
| — | 0% | 38.5 | 32.0 | 905 | 1.68 | 1.40 |
| CMG110 | 5% | 28.0 | 18.1 | 1788 | 2.77 | −28.53 |
| CMG110 | 1% | 38.5 | 10.5 | 1294 | 0.36 | −29.30 |
| CMG110 | 5% | 38.5 | 3.3 | 1242 | 0.32 | −36.57 |
| CMG31 | 1% | 38.5 | 16.7 | 963 | 1.35 | −16.40 |

^{a}Percent based on ratio of CMG primer mass to mass of expected alpha-1,3-glucan product (based on expected 45 g/L production by reaction).

As shown in Table 10, there was a dramatic difference in the zeta potential of non-CMG-primed alpha-1,3-glucan compared to the zeta potentials of alpha-1,3-glucan synthesized off CMG primer (i.e., CMGPG). The zeta potentials of CMGPG products in dispersion were more greatly negative compared to the zeta potentials of dispersed non-CMG-primed alpha-1,3-glucan products.

### Example 8 (Comparative)

### Carboxymethyl Cellulose Ether Does Not Successfully Prime Glucosyltransferase-Catalyzed Synthesis of Alpha-Glucan

This Example describes an attempt to use a glucosyltransferase to synthesize alpha-1,3-glucan graft copolymer using carboxymethyl cellulose (CMC) as a primer. This approach produced little, if any, CMC-primed alpha-1,3-glucan product, thereby indicating that cellulose ethers likely are not suitable for priming synthesis of alpha-1,3-glucan.

CMC was tested as a primer in glucosyltransferase priming reactions having largely the same conditions as described for the CMG70-primed reactions described in Example 5. Briefly, a series of three reactions was performed with about 10, 20, or 100 g/L sucrose, 5 mM sodium phosphate buffer (pH 5.5), 10 g/L of a CMC90 primer (CMC, DP ˜1540, DOS 0.9; Sigma-Aldrich, catalog no. 419303), and 100 U/L of a GTF enzyme. The GTF enzyme was the same as that used in Example 1. Another series of reactions was performed in the same manner, but using CMC70 (CMC, DP ˜560, DoS 0.7; Sigma-Aldrich, catalog no. 419273) as primer instead of CMC90. Since the CMC90 and CMC70 primers had DoS values similar to the DoS of primer CMG70, and the reaction conditions were almost the same as those tested with CMG70, the comparison in priming reactions was quite direct. In both reaction series using either CMC90 or CMC70 primer, increasing the sucrose concentration increased both alpha-1,3-glucan yield (glucosyl basis) and initial reaction rates. The highest glucan yields (˜80%) were observed in reactions with ˜100 g/L of sucrose, and more than 90% of sucrose was consumed after 24 hours in all the reactions. However, NMR analyses (data not shown) showed no obvious signs of CMC incorporation in the final insoluble glucan products of any of the reactions. The only exception to this observation was the glucan product of the reaction comprising CMC90 and ˜100 g/L sucrose; this product seemed to possibly have a trace amount of CMC incorporation. Instead of incorporation, though, this very low level CMC90 could be due to insufficient washing of the insoluble product (to remove residual CMC90 primer) prior to NMR testing. Regardless of the reason, the detected CMC90 level was insignificant as compared to the levels of CMG70 primer detected in insoluble products in Example 5. Additionally, the six reactions with CMC primers visually appeared to be the same as reactions that produce non-primed alpha-1,3-glucan (i.e., homopolymer); there was no dramatic viscosity increase or appearance changes as observed above with successful CMG priming reactions. All this evidence suggests that CMC does not prime, or does not appreciably prime, glucosyltransferase-catalyzed alpha-1,3-glucan synthesis.

1-18. (canceled)

19. A composition comprising a graft copolymer, wherein the graft copolymer comprises:
(A)
(i) a backbone comprising a dextran ether or ester compound that has a degree of substitution (DoS) of 0.001 to 3.0, wherein the dextran ether or ester compound comprises at least 90% alpha-1,6 glycosidic linkages, and
(ii) one or more alpha-1,3-glucan side chains comprising at least 90% alpha-1,3 glycosidic linkages, wherein the one or more side chains branch from said backbone; or
(B)
(i) a backbone comprising an alpha-1,3-glucan ether or ester compound that has a DoS of 0.001 to 3.0, wherein the alpha-1,3-glucan ether or ester compound comprises at least 30% alpha-1,3 linkages and a percentage of alpha-1,6 linkages that brings the total of both the alpha-1,3 and alpha-1,6 linkages in the backbone to 100%, and
(ii) one or more alpha-1,3-glucan side chains comprising at least 90% alpha-1,3 glycosidic linkages, wherein the one or more side chains branch from said backbone; or

20. The composition of claim 19, wherein the graft copolymer is said graft copolymer of (A).

21. The composition of claim 20, wherein the weight-average degree of polymerization (DPw) of the dextran ether or ester compound is at least 15.

22. The composition of claim 20, wherein the dextran ether or ester compound comprises at least 99% alpha-1,6 glycosidic linkages.

23. The composition of claim 20, wherein the backbone comprises the dextran ether compound.

24. The composition of claim 23, wherein the dextran ether compound comprises an ether-linked carboxymethyl group.

25. The composition of claim 20, wherein the one or more alpha-1,3-glucan side chains comprise at least 95% alpha-1,3 glycosidic linkages.

26. The composition of claim 20, wherein the DPw of said one or more alpha-1,3-glucan side chains is at least 100.

27. The composition of claim 19, wherein the graft copolymer is said graft copolymer of (B).

28. The composition of claim 27, wherein the DPw of the alpha-1,3-glucan ether or ester compound is at least 15.

29. The composition of claim 27, wherein the backbone comprises the alpha-1,3-glucan ether compound.

30. The composition of claim 29, wherein the alpha-1,3-glucan ether compound comprises an ether-linked carboxymethyl group.

31. The composition of claim 27, wherein the one or more alpha-1,3-glucan side chains comprise at least 95% alpha-1,3 glycosidic linkages.

32. The composition of claim 27, wherein the DPw of said one or more alpha-1,3-glucan side chains is at least 100.

33. The composition of claim 19, wherein the composition is an aqueous composition.

34. The composition of claim 20, wherein the graft copolymer is produced in a reaction composition comprising at least water, sucrose, said dextran ether or ester compound, and a glucosyltransferase enzyme that synthesizes alpha-1,3-glucan with at least 90% alpha-1,3 glycosidic linkages.

35. The composition of claim 27, wherein the graft copolymer is produced in a reaction composition comprising at least water, sucrose, said alpha-1,3-glucan ether or ester compound, and a glucosyltransferase enzyme that synthesizes alpha-1,3-glucan with at least 90% alpha-1,3 glycosidic linkages.

36. A method of producing a graft copolymer, said method comprising:
(a) contacting, in a reaction composition, at least (i) water, (ii) sucrose, (iii) an dextran ether or ester compound that has a DoS of 0.001 to 3.0, and (iv) a glucosyltransferase enzyme that synthesizes alpha-1,3-glucan comprising at least 90% alpha-1,3 glycosidic linkages,
whereby a graft copolymer according to the composition of claim 20 is produced; and
(b) optionally, isolating the graft copolymer produced in step (a).

37. A method of producing a graft copolymer, said method comprising:
(a) contacting, in a reaction composition, at least (i) water, (ii) sucrose, (iii) an alpha-1,3-glucan ether or ester compound that has a DoS of 0.001 to 3.0, and (iv) a glucosyltransferase enzyme that synthesizes alpha-1,3-glucan comprising at least 90% alpha-1,3 glycosidic linkages,
whereby a graft copolymer according to the composition of claim 27 is produced; and
(b) optionally, isolating the graft copolymer produced in step (a).
